(19) United States
(12) Patent Application Publication
(10) Pub. No.: US 2023/0069322 A1
(43) Pub. Date: Mar. 2, 2023

(54) COMPOSITIONS AND METHODS FOR GAMMA DELTA TCR REPROGRAMMING USING FUSION PROTEINS

(71) Applicant: TCR2 Therapeutics Inc., Cambridge, MA (US)

(72) Inventors: Jian DING, Newton, MA (US); Robert HOFMEISTER, Scituate, MA (US); Patrick Alexander BAEUERLE, Gauting (DE); Dario GUTIERREZ, Waltham, MA (US); Robert TIGHE, Cambridge, MA (US); Julio GOMEZ RODRIGUEZ, Cambridge, MA (US)

(21) Appl. No.: 17/788,411

(22) PCT Filed: Dec. 23, 2020
(86) PCT No.: PCT/US2020/066919

§ 371 Date: Jun. 23, 2022

Related U.S. Application Data
(60) Provisional application No. 62/953,382, filed on Dec. 24, 2019.

(51) Int. Cl. A61K 35/17; C07K 14/725; C07K 16/28; C07K 16/18; C07K 16/30; C07K 16/32; C07K 14/54; C07K 14/715; C07K 14/705; A61P 35/00; C12N 5/0783

(52) U.S. Cl. CPC A61K 35/17; C07K 14/7051; C07K 16/2803; C07K 16/2878; C07K 16/18; C07K 16/3092; C07K 16/32; C07K 16/3069; C07K 16/2875; C07K 16/2827; C07K 16/2842; C07K 14/5443; C07K 14/7155; C07K 14/70521; A61P 35/00; C12N 5/0638; A61K 38/00

(57) ABSTRACT

Provided herein are recombinant nucleic acids encoding T cell receptor (TCR) fusion proteins (TFPs), modified γδ T cells expressing the encoded molecules, and methods of use thereof for the treatment of diseases, including cancer.

### CROSS-REFERENCE

This application claims the benefit of U.S. Provisional Application No. 62/953,382, filed Dec. 24, 2019, which is incorporated herein by reference in its entirety.

### BACKGROUND OF THE INVENTION

Engineered autologous alpha beta (αβ) T cells expressing either T Cell Receptor Fusion Constructs (“TFPs”) or chimeric antigen receptors (CARs) at their surface have produced exciting anti-tumor responses in vitro and in vivo. However, one limitation of current autologous αβ T cell therapies is that the T cells must first be obtained from the subject being treated, engineered to express the TFP or CAR, and reintroduced into the subject, delaying treatment by multiple weeks, relative to off the shelf therapies.

### SUMMARY OF THE INVENTION

Provided herein are engineered gamma delta (γδ) T cells expressing T Cell Receptor Fusion Constructs (“TFPs”), methods of producing the modified T cells, and methods of use thereof in treatment. Advantageously, such cells can be naturally allogeneic. This is an improvement over current therapeutics produced with autologous alpha beta (αβ) T cells. Allogenic TFP expressing cells are an “off the shelf” therapy that overcome a number of limitations of αβ T cells expressing TFPs, including natural patient-to-patient immune variability that leads to profound differences in product potency, complex-personalized manufacturing with high cost of goods, multiple weeks between leukapheresis and treatment, lack of scalability, and poor solid tumor efficacy. In some embodiments, the γδ TCR complex having the integrated TFP functions cooperatively with a native γδ TCR in the γδ TCR.

In an aspect, the present disclosure provides a cell comprising a recombinant nucleic acid encoding a T cell receptor (TCR) fusion protein (TFP) comprising: (a) a TCR-integrating subunit comprising: (i) at least a portion of a TCR extracellular domain, (ii) a transmembrane domain, and (iii) an intracellular domain comprising a stimulatory domain from an intracellular signaling domain of CD3 epsilon, CD3 gamma, or CD3 delta, or CD3 zeta; and (b) an antibody or fragment thereof comprising an antigen binding domain; wherein the TCR-integrating subunit and the antibody are operatively linked, wherein the cell is a gamma delta (γδ) T cell, and wherein the expressed TFP functionally incorporates into a γδ TCR complex of the γδ T cell.

In some embodiments, wherein the antibody or fragment thereof is human or humanized. In some embodiments, the antibody or fragment thereof is murine. In some embodiments, the antibody or fragment thereof binds to a cell surface antigen. In some embodiments, the antibody or fragment thereof binds to a cell surface antigen on the surface of a tumor cell. In some embodiments, the antibody is a full length antibody. In some embodiments, the antibody fragment is a scFv, a single domain antibody domain, a V_{H} domain or a V_{L} domain.

In some embodiments, an antigen binding domain is selected from a group consisting of an anti-CD19 binding domain, anti-B-cell maturation antigen (BCMA) binding domain, anti-mesothelin (MSLN) binding domain, an anti-MUC16 binding domain, an anti-HER2 binding domain, an anti-PSMA binding domain, an anti-CD70 binding domain, an anti-CD79B binding domain, an anti-PD-L1 binding domain, an anti-Nectin-4 binding domain, an anti-Trop-2 binding domain, an anti-GPC3 binding domain, and an anti-BAFF receptor binding domain.

In another aspect, the present disclosure provides a cell comprising a recombinant nucleic acid encoding T cell receptor (TCR) fusion protein (TFP) comprising: (a) a TCR-integrating subunit comprising: (i) at least a portion of a TCR extracellular domain, (ii) a transmembrane domain, and (iii) an intracellular domain comprising a stimulatory domain from an intracellular signaling domain of CD3 epsilon, CD3 gamma, CD3 delta, or CD3 zeta; and (b) an antigen binding domain comprising a ligand or a fragment thereof; wherein the TCR-integrating subunit and the antigen binding domain are operatively linked, wherein the cell is a γδ T cell; and wherein the expressed TFP functionally incorporates into a γδ TCR complex of the γδ T cell.

In some embodiments, the ligand or fragment thereof is capable of binding to an antibody or fragment thereof. In some embodiments, the ligand is capable of binding an Fc domain of the antibody. In some embodiments, the ligand is capable of selectively binding an IgG1 antibody. In some embodiments, the binding is capable of specifically binding an IgG4 antibody. In some embodiments, the cell further comprises a nucleic acid sequence encoding an antibody or fragment thereof capable of being bound by the binding ligand. In some embodiments, the ligand or fragment thereof binds to a receptor or polypeptide expressed on the surface of a cell. In some embodiments, the ligand binds to the receptor of a cell. In some embodiments, the ligand binds to the polypeptide expressed on a surface of a cell. In some embodiments, the ligand or fragment thereof is a Natural Killer Group 2D (NKG2D) ligand or a fragment thereof. In some embodiments, the receptor or polypeptide expressed on a surface of a cell comprises a stress response receptor or polypeptide. In some embodiments, the receptor or polypeptide expressed on a surface of a cell is an MHC class I-related glycoprotein.

In some embodiments, the MHC class I-related glycoprotein is selected from the group consisting of MICA, MICB, RAET1E, RAET1G, ULBP1, ULBP2, ULBP3, ULBP4 and combinations thereof. In some embodiments, the ligand or fragment thereof is a monomer, a dimer, a trimer, a tetramer, a pentamer, a hexamer, a heptamer, an octomer, a nonamer, or a decamer. In some embodiments, the ligand or fragment thereof is a monomer or a dimer. In some embodiments, the antigen binding domain comprises a monomer, a dimer, a trimer, a tetramer, a pentamer, a hexamer, a heptamer, an octomer, a nonamer, or a decamer. In some embodiments, the antigen binding domain comprises a monomer or a dimer of the ligand or fragment thereof. In some embodiments, the antigen binding domain does not comprise an antibody or fragment thereof. In some embodiments, the antigen binding domain does not comprise a variable region. In some embodiments, the antigen binding domain does not comprise a CDR.

In some embodiments, the TCR-integrating subunit and the antibody domain or the antigen binding domain are operatively linked by a linker sequence. In some embodiments, the linker sequence comprises (G₄S)ₙ, wherein n=1 to 4.

In some embodiments, the transmembrane domain is a transmembrane domain from CD3 epsilon, CD3 gamma, CD3 delta, TCR gamma or TCR delta.

In some embodiments, the intracellular domain is derived from at least in part CD3 epsilon, only CD3 gamma, or only CD3 delta. In some embodiments, the TCR-integrating subunit comprises (i) at least a portion of a TCR extracellular domain, (ii) a TCR transmembrane domain, and (iii) a TCR intracellular domain, wherein at least two of (i), (ii), and (iii) are from the same TCR subunit. In some embodiments, the TCR extracellular domain comprises an extracellular domain or portion thereof of a protein selected from the group consisting of a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications. In some embodiments, the TCR-integrating subunit comprises a transmembrane domain comprising a transmembrane domain of a protein selected from the group consisting of a TCR gamma chain, a TCR delta chain, a CD3 zeta chain, a CD3 epsilon TCR subunit, a CD3 gamma TCR subunit, or a CD3 delta TCR subunit, functional fragments thereof, and amino acid sequences thereof having at least one but not more than 20 modifications. In some embodiments, the TCR-integrating subunit comprises a TCR intracellular domain comprising a stimulatory domain of a protein selected from an intracellular signaling domain of CD3 epsilon, CD3 gamma or CD3 delta, or an amino acid sequence having at least one modification thereto. In some embodiments, the TCR-integrating subunit comprises an intracellular domain comprising a stimulatory domain of a functional signaling domain of CD3 zeta, or an amino acid sequence having at least one modification thereto.

In some embodiments, the cell further comprises a sequence encoding a costimulatory domain. In some embodiments, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto. In some embodiments, the TCR-integrating subunit comprises an immunoreceptor tyrosine-based activation motif (ITAM) of a TCR-integrating subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the ITAM replaces an ITAM of CD3 gamma, CD3 delta, CD3 epsilon, or CD3 zeta. In some embodiments, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit. In some embodiments, the at least one but not more than 20 modifications thereto comprise a modification of an amino acid that mediates cell signaling or a modification of an amino acid that is phosphorylated in response to a ligand binding to the TFP.

In some embodiments, the γδ T cell is an allogenic T cell. In some embodiments, the γδ T cell is a Vδ1+Vδ2−γδ T cell. In some embodiments, the γδ T cell is a Vδ1−Vδ2+γδ T cell. In some embodiments, the γδ T cell is a Vδ1−Vδ2−γδ T cell.

In some embodiments, the antigen binding domain is an anti-CD19 binding domain and the TFP comprises the sequence of SEQ ID NO: 22, SEQ ID NO: 24, or SEQ ID NO: 33. In some embodiments, the antigen binding domain is an anti-MSLN binding domain and the TFP comprises the sequence of SEQ ID NO: 25, SEQ ID NO: 26, SEQ ID NO: 41, or SEQ ID NO: 34. In some embodiments, the antigen binding domain is an anti-CD70 binding domain and the TFP comprises the sequence of SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, or SEQ ID NO: 80. In some embodiments, the antigen binding domain comprises a variable domain comprising a light chain complementarity determining region 1 (LC CDR1), a LC CDR2, and a LC CDR3. In some embodiments, the LC CDR1 comprises the sequence of SEQ ID NO: 10, the LC CDR2 comprises the sequence of SEQ ID NO: 12, and the LC CDR3 comprises the sequence of SEQ ID NO: 14. In some embodiments, the antigen binding domain comprises a variable domain comprising a heavy chain complementarity determining region 1 (HC CDR1), a HC CDR2, and a HC CDR3. In some embodiments, the HC CDR1 comprises the sequence of SEQ ID NO: 16, the HC CDR2 comprises the sequence of SEQ ID NO: 18, and the HC CDR3 comprises the sequence of SEQ ID NO: 20. In some embodiments, the antigen binding domain comprises a variable domain comprising a complementarity determining region (CDR) 1 of SEQ ID NO: 35, CDR2 of SEQ ID NO: 36, and CDR3 of SEQ ID NO: 37. In some embodiments, the antigen binding domain comprises a variable domain comprising a CDR1 of SEQ ID NO: 38, CDR2 of SEQ ID NO: 39, and CDR3 of SEQ ID NO: 40. In some embodiments, the antigen binding domain comprises a variable domain comprising a CDR1 of SEQ ID NO: 41, CDR2 of SEQ ID NO: 42, and CDR3 of SEQ ID NO: 43. In some embodiments, the antigen binding domain comprises a variable domain comprising a CDR1 of SEQ ID NO: 45, CDR2 of SEQ ID NO: 46, and CDR3 of SEQ ID NO: 47. In some embodiments, the antigen binding domain comprises a variable domain comprising a CDR1 of SEQ ID NO: 49, CDR2 of SEQ ID NO: 50, and CDR3 of SEQ ID NO: 51. In some embodiments, the antigen binding domain comprises a variable domain comprising a CDR1 of SEQ ID NO: 53, CDR2 of SEQ ID NO: 54, and CDR3 of SEQ ID NO: 55. In some embodiments, the antigen binding domain comprises a variable domain comprising a CDR1 of SEQ ID NO: 57, CDR2 of SEQ ID NO: 58, and CDR3 of SEQ ID NO: 59. In some embodiments, the antigen binding domain comprises a variable domain comprising a CDR1 of SEQ ID NO: 61, CDR2 of SEQ ID NO: 62, and CDR3 of SEQ ID NO: 63. In some embodiments, the antigen binding domain comprises a variable domain comprising a CDR1 of SEQ ID NO: 65, CDR2 of SEQ ID NO: 66, and CDR3 of SEQ ID NO: 67. In some embodiments, the antigen binding domain comprises a variable domain comprising a CDR1 of SEQ ID NO: 69, CDR2 of SEQ ID NO: 70, and CDR3 of SEQ ID NO: 71. In some embodiments, the antigen binding domain comprises a variable domain comprising a CDR1 of SEQ ID NO: 73, CDR2 of SEQ ID NO: 74, and CDR3 of SEQ ID NO: 75. In some embodiments, the antigen binding domain comprises a variable domain comprising a CDR1 of SEQ ID NO: 77, CDR2 of SEQ ID NO: 78, and CDR3 of SEQ ID NO: 79.

In some embodiments, the cell further comprises a first nucleic acid encoding an inhibitory molecule that comprises a first polypeptide comprising at least a portion of an inhibitory molecule, associated with a second polypeptide comprising a positive signal from an intracellular signaling domain. In some embodiments, the inhibitory molecule comprises the first polypeptide comprising at least a portion of PD-1 and the second polypeptide comprising a costimulatory domain and primary signaling domain. In some embodiments, the recombinant nucleic acid comprises the first nucleic acid. In some embodiments, the cell further comprises a second nucleic acid encoding IL-15. In some embodiments, the recombinant nucleic acid comprises the second nucleic acid. In some embodiments, IL-15 and the TFP are expressed in frame from the recombinant nucleic acid. In some embodiments, IL-15 and the TFP, when expressed in frame from the recombinant nucleic acid, are separated by a self-cleaving peptide, e.g., T2A. In some embodiments, expression of IL-15 increases persistence of the cells. In some embodiments, the second polypeptide comprises at least a portion of CD28. In some embodiments, the first polypeptide comprises an extracellular domain and a transmembrane domain of PD-1 linked to an intracellular domain of CD28. In some embodiments, the cell further comprises a nucleic acid sequence encoding IL-15 polypeptide or a fragment thereof. In some embodiments, the IL-15 polypeptide is secreted when expressed in a T cell. In some embodiments, the IL-15 polypeptide comprises a sequence of SEQ ID NO: 81. In some embodiments, the nucleic acid sequence further encodes an IL-15 receptor (IL-15R) subunit or a fragment thereof. In some embodiments, the IL-15R subunit is IL-15R alpha (IL-15Rα). In some embodiments, the nucleic acid sequence encodes a fusion protein comprising the IL-15 polypeptide linked to the IL-15Rα subunit. In some embodiments, the fusion protein comprises a sequence of SEQ ID NO: 84. In some embodiments, the fusion protein is expressed on cell surface when expressed. In some embodiments, the fusion protein is secreted when expressed. In some embodiments, the cell further comprises a first nucleic acid sequence encoding IL-15 receptor alpha (IL-15Rα) polypeptide or a fragment thereof.

In some embodiments, the first nucleic acid sequence further encodes PD-1 or a fragment thereof. In some embodiments, the first nucleic acid sequence further encodes CD28 or a fragment thereof. In some embodiments, the first nucleic acid sequence encodes a fusion protein comprising an extracellular domain and a transmembrane domain of PD-1 linked to an intracellular domain of CD28 linked to IL-15Rα. In some embodiments, the fusion protein comprises a sequence of SEQ ID NO: 85 or SEQ ID NO: 86.

In some embodiments, the cell further comprises a second nucleic acid sequence encoding an IL-15 polypeptide or a fragment thereof. In some embodiments, the IL-15 polypeptide or the fragment thereof is secreted when expressed. In some embodiments, the antigen binding domain of the TFP is an anti-CD19 binding domain and the TFP has the sequence of SEQ ID NO: 31. In some embodiments, the antigen binding domain of the TFP is an anti-MSLN binding domain and the TFP has the sequence of SEQ ID NO: 32.

In another aspect, the present disclosure provides a recombinant nucleic acid described herein. In some embodiments, the nucleic acid is selected from the group consisting of a DNA and an RNA. In some embodiments, the nucleic acid is an mRNA. In some embodiments, the nucleic acid is circRNA.

In some embodiments, the recombinant nucleic acid comprises a nucleic acid analog, wherein the nucleic acid analog is not in an encoding sequence of the recombinant nucleic acid. In some embodiments, the recombinant nucleic acid further comprises a leader sequence. In some embodiments, the recombinant nucleic acid further comprises a promoter sequence. In some embodiments, the recombinant nucleic acid further comprises a sequence encoding a poly(A) tail. In some embodiments, the recombinant nucleic acid further comprises a 3′UTR sequence. In some embodiments, the nucleic acid is an isolated nucleic acid or a non-naturally occurring nucleic acid. In some embodiments, the nucleic acid is an in vitro transcribed nucleic acid.

In another aspect, the present disclosure provides a vector comprising the recombinant nucleic acid described herein. In some embodiments, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some embodiments, the vector is an AAV6 vector. In some embodiments, the vector further comprises a promoter. In some embodiments, the vector comprises the recombinant nucleic acid of claim 93 or 94. In some embodiments, the vector comprises SEQ ID NOs: 29 or 30. In some embodiments, the vector is an in vitro transcribed vector.

In another aspect, the present disclosure provides a circular RNA comprising the recombinant nucleic acid described herein.

In another aspect, the present disclosure provides a pharmaceutical composition comprising: the cell described herein; and a pharmaceutically acceptable carrier.

In another aspect, the present disclosure provides a method of producing a plurality of cells comprising the cell described herein comprising: (a) activating the γδ T cells isolated from PBMCs obtained from a donor by contacting the γδ T cells with (i) one or more immunomodulatory agents, and (ii) one or more cytokines; (b) transducing the γδ T cells with the viral vector of any one of claims 106-112; and (c) expanding the γδ T cells in the presence of the one or more cytokines.

In some embodiments, the method comprises obtaining the PBMCs from a donor. In some embodiments, the method comprises isolating γδ T cells from the PBMCs. In some embodiments, the γδ T cells are obtained by negative selection. In some embodiments, γδ T cells are obtained by positive selection. In some embodiments, the one or more immunomodulatory agents comprise concanavalin, an anti-CD3 antibody, an anti-CD28 antibody, an anti-TCR delta antibody, an anti-TCR gamma antibody, or a pan Gamma delta TCR antibody. In some embodiments, the anti-TCR delta antibody is an anti-TCR delta1 antibody. In some embodiments, the anti-TCR gamma antibody is an anti-TCR gamma9 antibody. In some embodiments, the anti-TCR delta antibody, the anti-TCR gamma antibody, the pan Gamma delta TCR antibody are plate bound.

In some embodiments, the one or more immunomodulatory agents comprise anti-CD3 and anti-CD28 antibodies, e.g., human anti-CD3 and anti-CD28 antibodies. In some embodiments, the anti-CD3 and the anti-CD28 antibodies are bead bound. In some embodiments, the anti-CD3 and the anti-CD28 antibodies are suspended in a matrix.

In some embodiments, the one or more cytokines are IL-2, IL-4, IL-7 or IL-15. In some embodiments, the one or more cytokines are IL-7 and IL-15. In some embodiments, the one or more cytokines are IL-2, IL-7, and IL-15. In some embodiments, the one or more cytokines are IL-2 and IL-4. In some embodiments, the one or more immunomodulatory agents further comprise a retronectin. In some embodiments, the retronectin is plate bound. In some embodiments, the one or more immunomodulatory agents comprise anti-CD3 and anti-CD28 antibodies, e.g., human anti-CD3 and anti-CD28 antibodies, and the one or more cytokines are IL-7 and IL-15. In some embodiments, the one or more immunomodulatory agents comprise anti-CD3 and anti-CD28 antibodies, e.g., human anti-CD3 and anti-CD28 antibodies, and the one or more cytokines are IL-2, IL-7, and IL-15. In some embodiments, the one or more immunomodulatory agents comprise a retronectin and anti-CD3 and anti-CD28 antibodies, e.g., human anti-CD3 and anti-CD28 antibodies, and the one or more cytokines are IL-7 and IL-15. In some embodiments, the anti-CD3 and the anti-CD28 antibodies are bead bound. In some embodiments, the anti-CD3 and the anti-CD28 antibodies are suspended in a matrix. In some embodiments, the one or more immunomodulatory agents comprise the concanavalin and the one or more cytokines are IL-2 and IL-4. In some embodiments, the one or more immunomodulatory agents comprise an anti-TCR delta1 antibody and the one or more cytokines are IL-2, IL-7, and IL-15. In some embodiments, the one or more immunomodulatory agents comprise an anti-TCR gamma9 antibody and the one or more cytokines are IL-2, IL-7, and IL-15. In some embodiments, the one or more immunomodulatory agents comprise a retronectin and the pan Gamma delta TCR antibody and the one or more cytokines are IL-2, IL-7, and IL-15. In some embodiments, the one or more immunomodulatory agents comprise a retronectin and an anti-TCR gamma9 antibody and the one or more cytokines are IL-2, IL-7, and IL-15. In some embodiments, the cells are activated in the presence of the immunomodulatory agents for 2, 3, 4, or 5 days and then expanded in the absence of the immunomodulatory agents.

In some embodiments, the immunomodulatory agent comprises an antigen-presenting cell (APC) that expresses a ligand of the antigen binding domain of the TFP. In some embodiments, the γδ T cells is contacted with the APC prior to transduction in the presence or absence of one or more additional immunomodulatory agents. In some embodiments, the γδ T cells is contacted with the APC after removal of the one or more additional immunomodulatory agents. In some embodiments, the γδ T cells is contacted with the APC in the absence of the one or more immunomodulatory agents.

In another aspect, the present disclosure provides a method of treating cancer in a subject in need thereof, the method comprising administering to the subject a pharmaceutical composition comprising (a) a modified γδ T cell produced according to the method described herein, and (b) a pharmaceutically acceptable carrier.

In another aspect, the present disclosure provides a method of treating cancer in a subject in need thereof, the method comprising administering to the subject a therapeutically effective amount of the pharmaceutical composition described herein.

In some embodiments, less cytokines are released in the subject compared a subject administered an effective amount of an unmodified control γδ T cell. In some embodiments, less cytokines are released in the subject compared a subject administered an effective amount of a modified αβ T cell comprising the recombinant nucleic acid described herein, the vector described herein, or the circRNA described herein. In some embodiments, the method comprises administering the pharmaceutical composition in combination with an agent that increases the efficacy of the pharmaceutical composition. In some embodiments, the method comprises administering the pharmaceutical composition in combination with an agent that ameliorates one or more side effects associated with the pharmaceutical composition. In some embodiments, the cancer is a solid cancer, a lymphoma or a leukemia. In some embodiments, the cancer is selected from the group consisting of renal cell carcinoma, breast cancer, lung cancer, ovarian cancer, prostate cancer, colon cancer, cervical cancer, brain cancer, liver cancer, pancreatic cancer, kidney and stomach cancer.

In another aspect, the present disclosure provides the cell, the recombinant nucleic acid, the vector, the circRNA, or the pharmaceutical composition, as described herein, for use as a medicament or in the preparation of a medicament.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a schematic diagram showing the procedure used to activate, transfect, and expand αβ T cells and three different approaches used to activate, transfect, and expand γδ T cells.

FIGS. 2A-2C are a series of graphs showing expansion and developmental state of γδ T cells using Approach 1 described in Example 1. FIG. 2A is a series of graphs showing the fold expansion of Vd2+ cells. FIG. 2B shows flow cytometry data demonstrating transduction efficacy of Vd2+ cells with CD19 TFP and MSLN TFP. FIG. 2C shows flow cytometry data demonstrating the progression of non-transduced cells and transduced cells from naïve through the central memory state, effector memory state, to TEMRA, on days 0, 4, 8, 12, and 14 post activation.

FIGS. 3A-3B are a series of graphs showing T cell activation of MSLN TFP and CD19 TFP expressing γδ T cells. FIG. 3A shows flow cytometry data demonstrating activation of MSLN TFP and CD19 TFP expressing (TRuC+) γδ T cells transduced via lentiviral transduction treated with medium alone, anti-pan γδ, anti-CD3ε, CD19_Fc, or MSLN_Fc. FIG. 3B shows flow cytometry data demonstrating activation of MSLN TFP expressing γδ T cells transduced via electroporation of mRNA treated with medium alone or MSLN_Fc. Cells were prepared using Approach 1 described in Example 1.

FIGS. 4A-4B are a series of graphs showing cytokine production in MSLN TFP and CD19 TFP expressing γδ T cells. FIG. 4A is a series of graphs showing levels of IL-2, IFN-γ, GM-CSF, and TNF-α in nontransduced, MSLN TFP (TC-210) and CD19 TFP (TC-110) expressing γδ T cells treated with medium alone, anti-pan γδ, anti-CD3ε, CD19_Fc, or MSLN_Fc in a first donor (Donor B). FIG. 4B is a series of graphs showing levels of IL-2, IFN-γ, GM-CSF, and TNF-α in nontransduced, MSLN TFP (TC-210) lentiviral transduced and MSLN TFP (TC-210) mRNA electroporated γδ T cells treated with medium alone, anti-pan γδ, anti-CD3ε, CD19_Fc, or MSLN_Fc in a second donor (Donor C). Cells were prepared using Approach 1 described in Example 1.

FIGS. 5A-5B are a series of graphs showing cytotoxicity of cells expressing MSLN TFP. FIG. 5A is a series of graphs showing cytotoxicity of luciferase expressing K562 cells with or without MSLN expression co-cultured with γδ T cells transduced or electroporated with MSLN TFP at the ratios shown with or without zoledronic acid (zometa). FIG. 5B is a series of graphs showing cytotoxicity of luciferase expressing MSTO cells with or without MSLN expression co-cultured with γδ T cells transduced MSLN TFP at the ratios shown with or without zometa treatment. Cells were prepared using Approach 1 described in Example 1.

FIGS. 6A-6B are a series of graphs showing cytokine production in MSLN TFP expressing γδ T cells. FIG. 6A is a series of graphs showing levels of IL-2, IFN-γ, GM-CSF, and TNF-α in γδ T cells transduced or electroporated with MSLN TFP co-cultured with luciferase expressing K562 cells with or without MSLN expression at the ratios shown with or without zometa treatment. FIG. 6B is a series of graphs showing levels of IL-2, IFN-γ, GM-CSF, and TNF-α in γδ T cells transduced with MSLN TFP co-cultured with luciferase expressing MSTO cells with or without MSLN expression at the ratios shown with or without zometa treatment. Cells were prepared using Approach 1 described in Example 1.

FIGS. 7A-7E are a series of graphs showing expansion, cellular subset, developmental state, and transduction efficacy of γδ T cells transduced with MSLN TFP using Approach 2 described in Example 1. FIG. 7A is a graph showing the fold expansion of γδ T cells. FIGS. 7B and 7C are a series of graphs showing the proportion of Vδ1−Vδ2−, Vδ1+Vδ2−, and Vδ1−Vδ2+ cells at 7, 11, 15 and 24 days post-activation for non-transduced (FIG. 7B) and transduced (FIG. 7C) cells. FIG. 7D shows flow cytometry data demonstrating the progression of non-transduced cells and transduced Vδ1−Vδ2−, Vδ1+Vδ2−, and Vδ1−Vδ2+ cells at 0, 7, 11, 15 and 24 days post-activation from naïve through the central memory state, effector memory state, to TEMRA. FIG. 7E shows flow cytometry data demonstrating lentiviral transduction efficacy of the MSLN TFP in Vδ1−Vδ2−, Vδ1+Vδ2−, and Vδ1−Vδ2+ cells at 15 days post-activation.

FIGS. 8A-8D are a series of graphs showing cytotoxicity of cells expressing MSLN TFP. FIGS. 8A and 8B show cytotoxicity of luciferase expressing MSTO cells without (FIG. 8A) or with (FIG. 8B) MSLN expression co-cultured with γδ T cells transduced MSLN TFP at the ratios shown with or without zometa at 15 days of co-culture. FIGS. 8C and 8D shows cytotoxicity of luciferase expressing MSTO cells without (FIG. 8C) or with (FIG. 8D) MSLN expression co-cultured with γδ T cells transduced MSLN TFP at the ratios shown with or without zometa at 24 days of co-culture. Cells were prepared using Approach 2 described in Example 1.

FIGS. 9A-9P are a series of graphs showing cytokine expression in MSLN TFP expressing γδ T cells. FIGS. 9A-9D show levels of levels of IFN-γ, IL-2, TNF-α, and GM-CSF, respectively, in luciferase expressing MSTO cells with MSLN expression co-cultured with γδ T cells transduced MSLN TFP at the ratios shown with or without zometa at 15 days of co-culture. FIGS. 9E-9H show levels of levels of IFN-γ, IL-2, TNF-α, and GM-CSF, respectively, in luciferase expressing MSTO cells without MSLN expression co-cultured with γδ T cells transduced MSLN TFP at the ratios shown with or without zometa at 15 days of co-culture. FIGS. 9I-9L show levels of levels of IFN-γ, IL-2, TNF-α, and GM-CSF, respectively, in luciferase expressing MSTO cells with MSLN expression co-cultured with γδ T cells transduced MSLN TFP at the ratios shown with or without zometa at 24 days of co-culture.

FIGS. 9M-9P show levels of levels of IFN-γ, IL-2, TNF-α, and GM-CSF, respectively, in luciferase expressing MSTO cells without MSLN expression co-cultured with γδ T cells transduced MSLN TFP at the ratios shown with or without zometa at 24 days of co-culture. Cells were prepared using Approach 2 described in Example 1.

FIGS. 10A-10F are a series of graphs showing expansion, viability, cellular subset, transduction efficacy, and developmental state of γδ T cells transduced with MSLN TFP using Approach 3 described in Example 1. FIG. 10A is a graph showing the fold expansion of γδ T cells transduced with MSLN TFP. FIG. 10B is a graph showing % viability of γδ T cells transduced with MSLN TFP. FIGS. 10C and 10D are a series of graphs showing the proportion of Vδ1−Vδ2−, Vδ1+Vδ2−, and Vδ1−Vδ2+ cells at 4, 8, 15 and 24 days post-activation for non-transduced (FIG. 10C) and transduced (FIG. 10D) cells. FIG. 10E shows flow cytometry data demonstrating lentiviral transduction efficacy of the MSLN TFP in Vδ1−Vδ2−, Vδ1+Vδ2−, and Vδ1−Vδ2+ cells at 15 days post-activation. FIG. 10F shows flow cytometry data demonstrating the progression of non-transduced cells and transduced Vδ1−Vδ2−, Vδ1+Vδ2−, and Vδ1−Vδ2+ cells at 0, 4. 8, 15 and 24 days post-activation from naïve through the central memory state, effector memory state, to TEMRA.

FIGS. 11A-11B are a series of graphs showing cytotoxicity of cells expressing MSLN TFP. FIG. 8A shows cytotoxicity of luciferase expressing MSTO cells with or without MSLN expression co-cultured with γδ T cells transduced MSLN TFP at the ratios shown with or without zometa at 15 days of co-culture. FIG. 8B shows cytotoxicity of luciferase expressing MSTO cells with or without MSLN expression co-cultured with γδ T cells transduced MSLN TFP at the ratios shown with or without zometa at 24 days of co-culture. Cells were prepared using Approach 3 described in Example 1.

FIGS. 12A-12N are a series of graphs showing cytokine expression in MSLN TFP expressing γδ T cells. FIGS. 12A-12C show levels of levels of GM-CSF, IFN-γ, and TNF-α, respectively, in luciferase expressing MSTO cells with or without MSLN expression co-cultured with undransduced γδ T cells at the ratios shown with or without zometa at 15 days of co-culture.

FIGS. 12D-12F show levels of levels of GM-CSF, IFN-γ, and TNF-α, respectively, in luciferase expressing MSTO cells with or without MSLN expression co-cultured with γδ T cells transduced with MSLN TFP at the ratios shown with or without zometa at 15 days of co-culture.

FIGS. 12G-12J show levels of levels of GM-CSF, IFN-γ, TNF-α, and IL-2, respectively, in luciferase expressing MSTO cells with or without MSLN expression co-cultured with undransduced γδ T cells at the ratios shown with or without zometa at 15 days of co-culture.

FIGS. 12K-12N show levels of levels of GM-CSF, IFN-γ, TNF-α, and IL-2, respectively, in luciferase expressing MSTO cells with or without MSLN expression co-cultured with undransduced γδ T cells at the ratios shown with or without zometa at 15 days of co-culture. Cells were prepared using Approach 3 described in Example 1.

FIG. 13 is a schematic illustration of the experiment performed in Example 8.

FIG. 14 is graph showing expansion of γδ and αβ T cells having the TFP constructs shown generated according to the methods described in Example 8.

FIG. 15 is a series of plots showing the TCRαβ, CD3, VD1, and Vd2 phenotype of γδ and αβ T cells used in Example 8 prior to activation.

FIGS. 16A and 16B are a series of plots showing transduction efficiency of γδ and αβ T cells with the TFP constructs shown generated according to the methods described in Example 8. FIG. 16A shows T cells activated under polyclonal conditions and FIG. 16B shows T cells activated under plate bound conditions.

FIGS. 17A-17B are a series of plots showing the Vd1 and Vd2 phenotype of the γδ T cells transduced with the constructs shown. FIG. 17A shows T cells activated under polyclonal conditions and FIG. 17B shows T cells activated under plate bound conditions.

FIGS. 18A-18B are a series of graphs showing target lysis and cytokine secretion by γδ and αβ T cells having the TFP constructs shown when co-cultured with MSLN-expressing target cells. FIG. 18A shows T cells activated under polyclonal conditions and FIG. 18B shows T cells activated under plate bound conditions.

FIG. 19 is graph showing expansion of γδ and αβ T cells having the TFP constructs shown generated according to the methods described in Example 9.

FIG. 20 is a series of plots showing transduction efficiency of γδ and αβ T cells with the TFP constructs shown generated according to the methods described in Example 9.

FIG. 21 is a series of plots showing the proportion of PD-1+ cells of the γδ and αβ T cells transduced with the TFP constructs shown.

FIG. 22 is a series of plots showing the proportion of VD1+ and VD2+ cells of the γδ and αβ T cells transduced with the TFP constructs shown.

FIG. 23 is a series of plots showing the memory phenotype of γδ T cells having the TFP constructs shown.

FIG. 24 is a series of plots showing the proportion of NKG2D+ cells of the γδ and αβ T cells transduced with the TFP constructs shown.

FIG. 25 is a series of graphs showing target lysis by γδ and αβ T cells having the TFP constructs shown when co-cultured with MSLN-expressing or control target cells.

FIGS. 26A-26B are a series of graphs showing cytokine secretion by γδ and αβ T cells having the TFP constructs shown when co-cultured with MSLN-expressing or control target cells. FIG. 26A shows cytokine levels for IL-2, IFN-γ, GM-CSF, TNF-α, and CCL4. FIG. 26B shows cytokine levels for IL-15, CCL3, and IL-17A.

### DETAILED DESCRIPTION OF THE INVENTION

Provided herein are engineered gamma delta (γδ) T cells expressing T Cell Receptor Fusion Constructs (“TFPs”), methods of producing the modified T cells, and methods of use thereof in treatment. Advantageously, such cells can be naturally allogeneic. This is an improvement over current therapeutics produced with autologous alpha beta (αβ) T cells. Allogenic TFP expressing cells are an “off the shelf” therapy that overcome a number of limitations of αβ T cells expressing TFPs, including natural patient-to-patient immune variability that leads to profound differences in product potency, complex-personalized manufacturing with high cost of goods, multiple weeks between leukapheresis and treatment, lack of scalability, and poor solid tumor efficacy.

Currently autologous cell transfer is used in cellular therapies employing T Cell Receptor Fusion Constructs (“TFPs”) or chimeric antigen receptors (CARs). Cells from donors are not used due to the high risk of graft versus host disease (GvHD) associated with using T cells from HLA-mismatched donors. Certain subsets of T cells, including gamma delta (γδ) T cells, that are abundant in mucosal organs and solid tissues, but have low abundance in the blood, have invariant public TCRs that recognize highly conserved receptors (e.g., CD1c, BTN3A1, or MR1). An allogenic T cell product from healthy donors that mitigates GvHD by natural HLA-independent antigen recognition, such as a γδ T cell expressing a TFP or CAR, can be used to overcome the multiple biological limitations associated with autologous T cell transplant, in addition to making manufacturing scalable and reducing costs. Moreover, it will be appreciated that the γδ T cells expressing a TFP, described herein, are able to integrate into and redirect γδ TCRs to target a tumor associated antigen (TAA) are able to function cooperatively with native undisturbed TCRs of the same cell or cell population that target a different TAA.

Disclosed herein are compositions and methods comprising T cell receptor Fusion Proteins (TFPs) and therapeutics comprising human γδ T cells engineered to express TFPs. Described herein are modified γδ T cells comprising fusion proteins of TCR-integrating subunits, including CD3 epsilon, CD3 gamma, CD3 delta, CD3 zeta, TCR gamma, and/or TCR delta chains, or portions thereof, with binding domains specific for cell surface antigens that have the potential to overcome limitations of existing approaches. These modified T cells may have the ability to kill target cells more efficiently than αβ or γδ CAR T cells or unmodified γδ T cells but release comparable or lower levels of pro-inflammatory cytokines. These modified T cells and methods of their use may represent an advantage for these cells relative to CARs because elevated levels of these cytokines have been associated with dose-limiting toxicities for adoptive CAR T therapies. In some embodiments, the γδ TCR complex having the integrated TFP functions cooperatively with a native γδ TCR in the γδ TCR to produce increased cytotoxicity and/or increased reduction in tumor volume.

Disclosed herein, in some embodiments, are recombinant nucleic acids comprising (a) a sequence encoding a T cell receptor (TCR) fusion protein (TFP) comprising (i) a TCR-integrating subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, or CD3 zeta, and (ii) an antibody domain or an antigen binding domain comprising a ligand or a fragment thereof; wherein the TCR-integrating subunit and the antibody domain or the antigen binding domain are operatively linked, and wherein the TFP functionally incorporates into a TCR complex when expressed in a T cell. In some embodiments, the ligand or a fragment thereof is capable of binding to an antibody or fragment thereof. In some embodiments, the ligand or a fragment thereof binds to a receptor or polypeptide expressed on the surface of a cell.

Disclosed herein, in some embodiments, are vectors comprising the recombinant nucleic acid disclosed herein.

Disclosed herein, in some embodiments, are modified allogenic T cells comprising the sequence encoding the TFP disclosed herein or a TFP encoded by the sequence of the nucleic acid disclosed herein.

Disclosed herein, in some embodiments, are pharmaceutical compositions comprising: (a) the modified T cells of the disclosure; and (b) a pharmaceutically acceptable carrier.

Disclosed herein, in some embodiments, are methods of producing the transduced T cell with the recombinant nucleic acid of the disclosure, or the vectors disclosed herein.

Disclosed herein, in some embodiments, are methods of treating cancer in a subject in need thereof, the method comprising administering to the subject a therapeutically effective amount of the pharmaceutical compositions disclosed herein.

Disclosed herein, in some embodiments, are methods of treating cancer in a subject in need thereof, the method comprising administering to the subject a pharmaceutical composition comprising (a) a modified T cell produced according to the methods disclosed herein; and (b) a pharmaceutically acceptable carrier.

### Certain Terminology

Before describing the present invention in detail, it is to be understood that this disclosure is not limited to particularly exemplified materials or process parameters as such may, of course, vary. It is also to be understood that the terminology used herein is for the purpose of describing particular embodiments of the disclosure only, and is not intended to be limiting of the use of alternative terminology to describe the present disclosure.

All publications, patents and patent applications cited herein, whether supra or infra, are hereby incorporated by reference in their entirety for all purposes.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising,” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. “Patients” are subjects suffering from or at risk of developing a disease, disorder or condition or otherwise in need of the compositions and methods provided herein. In some embodiments, the subject has cancer, e.g., a cancer described herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition, and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999))

As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell.

As is used herein, the terms “T cell receptor” and “T cell receptor complex” are used interchangeably to refer to a molecule found on the surface of T cells that is, in general, responsible for recognizing antigens. The TCR comprises a heterodimer consisting of an alpha and beta chain in 95% of T cells, whereas 5% of T cells have TCRs consisting of gamma and delta chains. The TCR further comprises a combination of components of the CD3 complex. In some embodiments, the TCR comprises CD3ε. In some embodiments, the TCR comprises CD3γ. In some embodiments, the TCR comprises CD3. In some embodiments, the TCR comprises CD3δ. Engagement of the TCR with antigen, e.g., with antigen and WIC, results in activation of its T cells through a series of biochemical events mediated by associated enzymes, co-receptors, and specialized accessory molecules.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MEW molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

“Major histocompatibility complex (MEW) molecules are typically bound by TCRs as part of peptide:MHC complex. The MEW molecule may be an MEW class I or II molecule. The complex may be on the surface of an antigen presenting cell, such as a dendritic cell or a B cell, or any other cell, including cancer cells, or it may be immobilised by, for example, coating on to a bead or plate.

The human leukocyte antigen system (HLA) is the name of the gene complex which encodes major histocompatibility complex (MEW) in humans and includes HLA class I antigens (A, B & C) and HLA class II antigens (DP, DQ, & DR). HLA alleles A, B and C present peptides derived mainly from intracellular proteins, e.g., proteins expressed within the cell.

During T cell development in vivo, T cells undergo a positive selection step to ensure recognition of self MHCs followed by a negative step to remove T cells that bind too strongly to MHC which present self-antigens. As a consequence, certain T cells and the TCRs they express will only recognise peptides presented by certain types of MHC molecules—i.e. those encoded by particular HLA alleles. This is known as HLA restriction.

One HLA allele of interest is HLA-A*0201, which is expressed in the vast majority (>50%) of the Caucasian population. Accordingly, TCRs which bind WT1 peptides presented by MHC encoded by HLA-A*0201 (i.e. are HLA-A*0201 restricted) are advantageous since an immunotherapy making use of such TCRs will be suitable for treating a large proportion of the Caucasian population.

Other HLA-A alleles of interest are HLA-A*0101, HLA-A*2402, and HLA-A*0301.

Widely expressed HLA-B alleles of interest are HLA-B*3501, HLA-B*0702 and HLA-B*3502.

An “intracellular signaling domain,” as the term is used herein, refers to an intracellular portion of a molecule. The intracellular signaling domain generates a signal that promotes an immune effector function of the TFP containing cell, e.g., a modified T-T cell. Examples of immune effector function, e.g., in a modified T-T cell, include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation.

A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class 1 molecule, BTLA and a Toll ligand receptor, as well as OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

The term “antibody,” as used herein, refers to a protein, or polypeptide sequences derived from an immunoglobulin molecule, which specifically binds to an antigen. Antibodies can be intact immunoglobulins of oligoclonal or monoclonal origin, or fragments thereof and can be derived from natural or from recombinant sources.

The terms “antibody fragment” refers to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)₂, and Fv fragments, single-chain (sc)Fv (“scFv”) antibody fragments, linear antibodies, single domain antibodies such as sdAb (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “V_{H}” with regard to an antibody refers to the fragment of the heavy chain that contains three CDRs interposed between flanking stretches known as framework regions, these framework regions are generally more highly conserved than the CDRs and form a scaffold to support the CDRs. A camelid “V_{H}H” domain is a heavy chain comprising a single variable antibody domain.

Unless specified, as used herein a scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The portion of the TFP composition of the disclosure comprising an antibody or antibody fragment thereof may exist in a variety of forms where the antigen binding domain is expressed as part of a contiguous polypeptide chain including, for example, a single domain antibody fragment (sdAb), a single chain antibody (scFv) derived from a murine, humanized or human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a TFP composition of the disclosure comprises an antibody fragment. In a further aspect, the TFP comprises an antibody fragment that comprises a scFv or a sdAb.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response. This immune response may involve either antibody production, or the activation of specific immunologically-competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

As used herein, the term “CD19” refers to the Cluster of Differentiation 19 protein, which is an antigenic determinant detectable on B cell leukemia precursor cells, other malignant B cells and most cells of the normal B cell lineage.

As used herein, the term “BCMA” refers to the B-cell maturation antigen also known as tumor necrosis factor receptor superfamily member 17 (TNFRSF17) and Cluster of Differentiation 269 protein (CD269) is a protein that in humans is encoded by the TNFRSF17 gene. TNFRSF17 is a cell surface receptor of the TNF receptor superfamily which recognizes B-cell activating factor (BAFF) (see, e.g., Laabi et al., EMBO 11 (11): 3897-904 (1992). This receptor is expressed in mature B lymphocytes, and may be important for B-cell development and autoimmune response.

As used herein, the term “CD16” (also known as FcγRIII) refers to a cluster of differentiation molecule found on the surface of natural killer cells, neutrophil polymorphonuclear leukocytes, monocytes and macrophages. CD16 has been identified as Fc receptors FcγRIIIa (CD16a) and FcγRIIIb (CD16b), which participate in signal transduction. CD16 is a molecule of the immunoglobulin superfamily (IgSF) involved in antibody-dependent cellular cytotoxicity (ADCC).

“NKG2D,” as used herein, refers to a transmembrane protein belonging to the CD94/NKG2 family of C-type lectin-like receptors. In humans, NKG2D is expressed by NK cells, γδ T cells and CD8+αβ T cells. NKG2D recognizes induced-self proteins from MIC and RAET1/ULBP families which appear on the surface of stressed, malignant transformed, and infected cells.

“Mesothelin” (MSLN) refers to a tumor differentiation antigen that is normally present on the mesothelial cells lining the pleura, peritoneum and pericardium. Mesothelin is over expressed in several human tumors, including mesothelioma and ovarian and pancreatic adenocarcinoma. scFV and single domain antibodies suitable for use with the methods disclosed herein is described, e.g., in WO2018067993, which is incorporated by reference herein in it's entirety.

Tyrosine-protein kinase transmembrane receptor “ROR1”, also known as neurotrophic tyrosine kinase, receptor-related 1 (NTRKR1) is a member of the receptor tyrosine kinase-like orphan receptor (ROR) family. It plays a role in metastasis of cancer.

The term “MUC16”, also known as “mucin 16, cell-surface associated” or “ovarian cancer-related tumor marker CA125” is a membrane-tethered mucin that contains an extracellular domain at its amino terminus, a large tandem repeat domain, and a transmembrane domain with a short cytoplasmic domain. Products of this gene have been used as a marker for different cancers, with higher expression levels associated with poorer outcomes.

The “CD79α” and “CD79β” genes encode proteins that make up the B lymphocyte antigen receptor, a multimeric complex that includes the antigen-specific component, surface immunoglobulin (Ig). Surface Ig non-covalently associates with two other proteins, Ig-alpha and Ig-beta (encoded by CD79α and its paralog CD79β, respectively) which are necessary for expression and function of the B-cell antigen receptor. Functional disruption of this complex can lead to, e.g., human B-cell chronic lymphocytic leukemias.

B cell activating factor, or “BAFF” is a cytokine that belongs to the tumor necrosis factor (TNF) ligand family. This cytokine is a ligand for receptors TNFRSF13B/TACI, TNFRSF17/BCMA, and TNFRSF13C/BAFF-R. This cytokine is expressed in B cell lineage cells, and acts as a potent B cell activator. It has been also shown to play an important role in the proliferation and differentiation of B cells.

“CD70” is a is a cytokine that belongs to the tumor necrosis factor (TNF) ligand family. This cytokine is a ligand for TNFRSF27/CD27. It is a surface antigen on activated, but not on resting, T and B lymphocytes. CD70 induces proliferation of costimulated T cells, enhances the generation of cytolytic T cells, and contributes to T cell activation. CD70 is also reported to play a role in regulating B-cell activation, cytotoxic function of natural killer cells, and immunoglobulin synthesis.

“Prostate-specific membrane antigen” (PSMA) is a type II membrane protein expressed in all forms of prostate tissue, including carcinoma. The PSMA protein has a unique 3-part structure: a 19-amino-acid internal portion, a 24-amino-acid transmembrane portion, and a 707-amino-acid external portion. PSMA acts as a glutamate-preferring carboxypeptidase. PMSA expression is increased in cancer tissue in the prostate.

“HER2” encodes a member of the epidermal growth factor (EGF) receptor family of receptor tyrosine kinases. This protein has no ligand binding domain of its own and therefore cannot bind growth factors. However, it does bind tightly to other ligand-bound EGF receptor family members to form a heterodimer, stabilizing ligand binding and enhancing kinase-mediated activation of downstream signalling pathways, such as those involving mitogen-activated protein kinase and phosphatidylinositol-3 kinase. Amplification and/or overexpression of this gene has been reported in numerous cancers, including breast and ovarian tumors. Alternative splicing results in several additional transcript variants, some encoding different isoforms and others that have not been fully characterized. HER2 is amplified and/or overexpressed in 20-30% of invasive breast carcinomas. HER2-positive breast cancer is treated in a separate manner from other subtypes of breast cancer and commonly presents as more aggressive disease.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place.

The term “autologous” refers to any material derived from the same individual to whom it is later to be re-introduced into the individual.

The term “allogeneic” or, alternatively, “allogenic,” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically. In some embodiments, the allogeneic cells are T cells. In some embodiments, the T cells have naturally invariant TCRs that recognize the same receptor (antigen) in every individual in an HLA-independent manner, making them naturally allogeneic. In some embodiments, the allogeneic cells are γδ T cells, e.g., human γδ T cells. In some embodiments, the allogeneic cells are γ9δ2, γ2δ1, γ3δ1, γ4δ1, γ5δ1, γ8δ1, γ9δ1, γ10δ1, δ3 and δ5 subsets of γδ T cells, e.g., human γδ T cells. In some embodiments, the allogeneic cells are αβT cell subsets such as Vα24-Jα18 β11 (iNKT).

The term “xenogeneic” refers to a graft derived from an animal of a different species.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

The term “effective amount” or “therapeutically effective amount” are used interchangeably herein, and refer to an amount of a compound, formulation, material, or composition, as described herein effective to achieve a particular biological or therapeutic result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “functional disruption” refers to a physical or biochemical change to a specific (e.g., target) nucleic acid (e.g., gene, RNA transcript, of protein encoded thereby) that prevents its normal expression and/or behavior in the cell. In one embodiment, a functional disruption refers to a modification of the gene via a gene editing method. In one embodiment, a functional disruption prevents expression of a target gene (e.g., an endogenous gene).

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR™ gene delivery technology from Oxford BioMedica, the LENTIMAX™ vector system from Lentigen, and the like. Nonclinical types of lentiviral vectors are also available and would be known to one skilled in the art.

The term “circularized RNA” or “circRNA” refers to a class of single-stranded RNAs with a contiguous structure that have enhanced stability and a lack of end motifs necessary for interaction with various cellular proteins. CircRNAs are 3-5′ covalently closed RNA rings, and circRNAs do not display Cap or poly(A) tails. CircRNAs lack the free ends necessary for exonuclease-mediated degradation, rendering them resistant to several mechanisms of RNA turnover and granting them extended lifespans as compared to their linear mRNA counterparts. For this reason, circularization may allow for the stabilization of mRNAs that generally suffer from short half-lives and may therefore improve the overall efficacy of mRNA in a variety of applications. CircRNAs are produced by the process of splicing, and circularization occurs using conventional splice sites mostly at annotated exon boundaries (Starke et al., 2015; Szabo et al., 2015). For circularization, splice sites are used in reverse: downstream splice donors are “backspliced” to upstream splice acceptors (see Jeck and Sharpless, 2014; Barrett and Salzman, 2016; Szabo and Salzman, 2016; Holdt et al., 2018 for review).

Three general strategies have been reported so far for RNA circularization: chemical methods using cyanogen bromide or a similar condensing agent, enzymatic methods using RNA or DNA ligases, and ribozymatic methods using self-splicing introns. In preferred embodiments, precursor RNA is synthesized by run-off transcription and then heated in the presence of magnesium ions and GTP to promote circularization. RNA so produced can efficiently transfect different kinds of cells. In one aspect, the template includes sequences for the TFP, CAR, and TCR, or combination thereof.

In some exemplary embodiments, a ribozymatic method utilizing a permuted group I catalytic intron is used. This method is more applicable to long RNA circularization and requires only the addition of GTP and Mg2+ as cofactors. This permuted intron-exon (PIE) splicing strategy consists of fused partial exons flanked by half-intron sequences. In vitro, these constructs undergo the double transesterification reactions characteristic of group I catalytic introns, but because the exons are fused, they are excised as covalently 5′ and 3′linked circles.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Human” or “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the present disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TFP of the present disclosure can be replaced with other amino acid residues from the same side chain family and the altered TFP can be tested using the functional assays described herein.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n-1, n-2, n-3, n-4, n-5, n-6, n-7, n-8, n-9 and n-10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). Also included within the scope of the present disclosure are linkers described in WO2012/138475 (incorporated herein by reference). In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present disclosure, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present disclosure are derived from, cancers including but not limited to primary or metastatic melanoma, thymoma, lymphoma, sarcoma, lung cancer, liver cancer, NHL, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, an antibody fragment or a specific ligand, which recognizes and binds a cognate binding partner (e.g., CD19) present in a sample, but which does not necessarily and substantially recognize or bind other molecules in the sample.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### Compositions

Provided herein are recombinant nucleic acids encoding T cell receptor (TCR) fusion proteins (TFPs). Provided herein are γδ T cells comprising a recombinant nucleic acid encoding a T cell receptor (TCR) fusion protein (TFP), as described herein. In some embodiments, the γδ T cells are allogeneic. In some embodiments, the allogeneic γδ T cells include but are not limited to the Vδ2+, Vδ1+, and Vδ2−Vδ1− subsets.

Gamma delta (γδ) T cells are the prototype of ‘unconventional’ T cells and represent a relatively small subset of T cells in peripheral blood. They are defined by expression of heterodimeric T-cell receptors (TCRs) composed of γ and δ chains. This sets them apart from the classical and much better known αβ TCRs. The mechanism of (thymic) selection of γδ T cells is still largely unknown.

γδ T cells often show tissue-specific localisation of oligoclonal subpopulations sharing the same TCR chains. For instance, human peripheral blood γδ T cells are largely Vγ9/Vδ2+, and murine skin γδ T cells, so-called dendritic epidermal T cells (DETCs), are largely Vγ5/Vδ1+. In general, γδ T cells are enriched in epithelial and mucosal tissues where they are thought to serve as the first line of defense against pathogenic challenge.

The majority of γδ T cells are activated in an MHC-independent manner, in striking contrast to MHC-restricted αβ T cells. The antigens recognized by most γδ T cells are still unknown. A small proportion of murine γδ T cells (<1%) bind the MHC-I-related proteins T10 and T22 that are expressed by highly activated cells. Human Vγ9/Vδ2+T cells show TCR-dependent activation by certain phosphorylated metabolites such as microbial HMB-PP or eukaryotic isoprenoid precursor IPP. Due to metabolic dysregulation IPP is often accumulated by cancer cells. Some γδ T cells also recognise markers of cellular stress, resulting from infection or tumorigenesis. Stress surveillance performed by γδ T cells is thought to depend not only on their TCRs but also on co-stimulatory signals from, for instance, NK-type receptors. Finally, γδ TCRs have been shown to recognise lipid antigens presented by CD1 molecules, in particular CD1d.

Unlike conventional αβT cells, γδ T cells are largely tissue resident and have been shown to be resistant to hypoxic conditions; in fact their cytotoxic potential is largely enhanced by low oxygen levels, making them naturally adept to use in solid tumor cell therapy (Siegers et al., Front. Immunol. 9:1367 (2018)).

γδ T cells have natural tumor cell recognition and killing through expression of their TCR, NKG2D, NKp30, CD16, and NKp44, that can prevent escape of tumor cells with low or no expression of the targeted tumor antigen (Silva-Santos et al., Nature Reviews Immunology November; 15(11):683-91 (2015)). γδ T cell abundance in solid and heme tumors has been found to be a strong favorable prognostic for patient outcome (Gentles et al., Nature Medicine 21(8): 938-945 (2015)). Multiple studies have found that γδ T cell abundance in recipients of HSC transplants strongly correlates with overall survival and progression free survival due to their innate graft-versus-leukemia activity and lack of GvHD (Minculescu et al., Frontiers in Immunology 0:1997 (2019)).

Similar to T cell receptor complexes in alpha beta (αβ) T cells, the gamma delta (γδ) TCRs can be fused with tumor-antigen-binding motifs (such as the TFPs disclosed herein and variations thereof) to the CD3ε, CD3γ or CD3δ subunits. In comparison to conventional αβ TFP T cells, the γδ TFP T cells contemplated herein (and variations thereof) can be used for allogeneic cell therapy without risk for GvHD, and are predicted to provide increased solid tumor efficacy, and decreased rare tumor cell escape due to downregulation of the tumor antigen. In comparison to unmodified γδ T cells, γδ TFP T cells have increased specificity and additional recognition to prevent natural escape mechanisms that tumors have evolved to evade γδ T cells (i.e., BTN3A1 downregulation, and B7H6 and MICAS shedding). γδ TFP T cells have additive/synergistic cytotoxity that is mediated via tumor antigen recognition by the TFP and BTN3A1-mediated phosphotangtigen recognition by Vγ9/Vδ2 T cell receptor, potentially making them more potent than unmodified γδ T cells and than TFP αβ T cells.

### T Cell Receptor (TCR) Fusion Proteins (TFPs)

Provided herein are compositions of matter and methods of use for the treatment of a disease such as cancer, using modified γδ T cells comprising a T cell receptor (TCR) fusion protein. As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell.

As provided herein, TFPs provide substantial benefits as compared to Chimeric Antigen Receptors. The term “Chimeric Antigen Receptor” or alternatively a “CAR” refers to a recombinant polypeptide comprising an extracellular antigen binding domain in the form of, e.g., a single domain antibody or scFv, a transmembrane domain, and cytoplasmic signaling domains (also referred to herein as “intracellular signaling domains”) comprising a functional signaling domain derived from a stimulatory molecule as defined below. Generally, the central intracellular signaling domain of a CAR is derived from the CD3 zeta chain that is normally found associated with the TCR complex. The CD3 zeta signaling domain can be fused with one or more functional signaling domains derived from at least one co-stimulatory molecule such as 4-1BB (i.e., CD137), CD27 and/or CD28.

The present disclosure encompasses recombinant DNA constructs encoding TFPs, wherein the TFP comprises a binding domain, e.g., an antibody or antibody fragment, a ligand, or a ligand binding protein, wherein the sequence of the binding domain is contiguous with and in the same reading frame as a nucleic acid sequence encoding a TCR-integrating subunit or portion thereof. The TFPs provided herein are able to associate with one or more endogenous (or alternatively, one or more exogenous, or a combination of endogenous and exogenous) TCR subunits in order to form a functional TCR complex.

In one aspect, the TFP of the present disclosure comprises a target-specific binding element otherwise referred to as an antigen binding domain. The choice of moiety depends upon the type and number of target antigen that define the surface of a target cell. For example, the antigen binding domain may be chosen to recognize a target antigen that acts as a cell surface marker on target cells associated with a particular disease state. Thus examples of cell surface markers that may act as target antigens for the antigen binding domain in a TFP of the present disclosure include those associated with viral, bacterial and parasitic infections; autoimmune diseases; and cancerous diseases (e.g., malignant diseases).

In one aspect, the TFP-mediated T cell response can be directed to an antigen of interest by way of engineering an antigen-binding domain into the TFP that specifically binds a desired antigen.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise a natural or synthetic ligand specifically recognizing and binding a target antigen can be used as antigen binding domain for the TFP. In some instances, it is beneficial for the antigen binding domain to be derived from the same species in which the TFP will ultimately be used in. For example, for use in humans, it may be beneficial for the antigen binding domain of the TFP to comprise human or humanized residues for the antigen binding domain of an antibody or antibody fragment.

Thus, in one aspect, the TFP comprises an antigen-binding domain comprises a humanized or human antibody or an antibody fragment, or a murine antibody or antibody fragment. In one embodiment, the murine, human, or humanized antigen binding domain is, e.g., an antibody or antibody fragment. In one embodiment, the antibody or antibody fragment is an scFv or single domain antibody (sdAb). In one embodiment, the antibody is an scFv and comprises one or more (e.g., all three) light chain complementary determining region 1 (LC CDR1), light chain complementary determining region 2 (LC CDR2), and light chain complementary determining region 3 (LC CDR3) of a humanized or human anti-tumor-associated antigen binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a murine, humanized or human anti-tumor-associated antigen binding domain described herein, e.g., a murine, humanized or human anti-CD19, anti-BCMA, anti-MUC16, anti-mesothelin (anti-MSLN), anti-CD79B, anti-HER2, anti-PSMA, anti-CD70 binding domain comprising one or more, e.g., all three, LC CDRs and one or more, e.g., all three, HC CDRs. In some cases, the antibody or antibody fragment is a single domain antibody and comprises a variable domain having a CDR1, CDR2, and CDR3. For example, the single domain antibody can comprise a variable domain having a CDR1, CDR2, and CDR3 of an anti-CD19 binding domain, anti-B-cell maturation antigen (BCMA) binding domain, anti-mesothelin (MSLN) binding domain, an anti-MUC16 binding domain, an anti-HER2 binding domain, an anti-PSMA binding domain, an anti-CD70 binding domain, an anti-CD79B binding domain, an anti-PD-L1 binding domain, an anti-Nectin-4 binding domain, an anti-Trop-2 binding domain, an anti-GPC3 binding domain, or anti-BAFF receptor binding domain.

In one embodiment, the humanized or human anti-tumor-associated antigen (anti-TAA) binding domain comprises a humanized heavy chain variable region described herein, e.g., at least two humanized or human heavy chain variable regions described herein. In one embodiment, the anti-TAA binding domain is a scFv comprising a light chain and a heavy chain of an amino acid sequence provided herein. In an embodiment, the anti-TAA binding domain (e.g., a scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the anti-TAA binding domain is a scFv, and a light chain variable region comprising an amino acid sequence described herein, is attached to a heavy chain variable region comprising an amino acid sequence described herein, via a linker, e.g., a linker described herein. In one embodiment, the humanized anti-TAA binding domain includes a (Gly₄-Ser)ₙ linker, wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region. In some instances, the linker sequence comprises (G₄S)ₙ, wherein n=2 to 4. In some instances, the linker sequence comprises (G₄S)ₙ, wherein n=1 to 3.

In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, Proc. Natl. Acad. Sci., 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology 28(4/5):489-498; Studnicka et al., Protein Engineering 7(6):805-814 (1994); and Roguska et al., Proc. Natl. Acad. Sci. 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al., Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a V_{H}4-4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3-1.25 germline sequence. In one embodiment, the framework region can comprise, one, two, three, four or five modifications, e.g., substitutions, e.g., from the amino acid at the corresponding murine sequence.

In some aspects, the portion of a TFP composition of the present disclosure that comprises an antibody fragment is humanized with retention of high affinity for the target antigen and other favorable biological properties. According to one aspect of the present disclosure, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present disclosure, the ability to bind a human TAA. In some embodiments, a humanized antibody or antibody fragment may have improved affinity and/or specificity of binding to human CD19, human BCMA, human MUC16, human mesothelin, human CD79B, human HER2, human PSMA, human CD70.

In one aspect, the binding domain is characterized by particular functional features or properties of an antibody or antibody fragment. For example, in one aspect, the portion of a TFP composition of the present disclosure that comprises an antigen binding domain specifically binds human CD19, human BCMA, human MUC16, human mesothelin, human CD79B, human HER2, human PSMA, human CD70.

In one aspect, the antigen binding domain has the same or a similar binding specificity to human CD19 as the FMC63 scFv described in Nicholson et al., Mol. Immun. 34 (16-17): 1157-1165 (1997). In one embodiment, the antibody has the antigen binding domain of FMC63 or another anti-CD19 antibody. Other exemplary antibodies that bind CD19 include, but are not limited to, inebilizumab, MDX-1342, tafasitamab, obexelimab, B4 (Merck), immunomedics hA19, and those described in WO2019112347, WO2010142952, WO2018108106, WO2009086514, WO2006121852, WO2010095031, WO2016059253, WO2009102473, WO2001058916, WO2012010561, WO2005012493, WO2008031056, WO2019214332, WO2017126587, WO2000035409, WO2019137518, WO2007002223, WO2012067981, WO2007076950, WO2002020615, WO2001070266, WO2005035582, WO2015179236, WO2013184218, WO2018101448, WO2016112855, WO2010021697, WO2001057226, WO2017165125, WO2016079276, WO2011147834, WO2006103100, WO2017015783, WO2013192596, WO2013024095, WO2008022152, WO2007115713, WO2012057765, and WO2019057100, the contents of each of which are incorporated by reference herein in their entirety.

In one embodiment, the antibody has the antigen binding domain of an anti-BCMA antibody. Exemplary antibodies that bind BCMA include, but are not limited to, SEA-BCMA (Seattle Genetics) and those described in WO2010104949, WO2011108008, WO2014122143, WO2016090327, WO2017143069, WO2017211900, WO2018133877, WO2019066435, WO2019149269. WO2019190969, and WO2019195017, the contents of each of which are incorporated by reference herein in their entirety.

In one embodiment, the antibody has the antigen binding domain of an anti-MUC16 antibody. Exemplary antibodies that bind MUC16 include, but are not limited to, oregovomab, 4H11 (Memorial Sloan Kettering Cancer Center), sofituzumab, and those described in WO2018058003, the contents of which is incorporated by reference herein in its entirety.

In one embodiment, the antibody has the antigen binding domain of an anti-mesothelin antibody. Exemplary antibodies that bind mesothelin include, but are not limited to, amatuximab and those described in WO2006099141, WO2006124641, WO2009120769, WO2010111282, WO2014004549, WO2014031476, WO2014052064, WO2017032293, and WO2017052241, the contents of each of which are incorporated by reference herein in their entirety.

In one embodiment, the antibody has the antigen binding domain of an anti-CD79B antibody. Exemplary antibodies that bind CD79B include, but are not limited to, those described in WO2017009474 and WO2016021621, the contents of each of which are incorporated by reference herein in their entirety.

In one embodiment, the antibody has the antigen binding domain of an anti-HER2 antibody. Exemplary antibodies that bind HER2 include, but are not limited to, trastuzumab, pertuzumab, margetuximab, trastuzumab-pkrb, ertumaxomab, SB3, PF-05280014, CMAB302, trastuzumab-dkst, HD201, GB221, BCD-022, trastuzumab-anns, HLX02, DMB-3111, timigutuzumab, UB-921, IBI315, RG6194, HLX22, SIBP-01, TX05, and DXL702.

In one embodiment, the antibody has the antigen binding domain of an anti-PSMA antibody. Exemplary antibodies that bind PSMA include, but are not limited to, MDX1201-A488 and those described in WO2001009192, WO200303490, WO2007002222, WO2009130575, WO2010118522, WO2013185117, WO2013188740, WO2014198223, WO2016145139, WO2017121905, WO2017180713, WO2017212250, WO2018033749, WO2018129284, WO2018142323, and WO2019191728, the contents of each of which are incorporated by reference herein in their entirety.

In one embodiment, the antibody has the antigen binding domain of an anti-CD70 antibody. Exemplary antibodies that bind CD70 include, but are not limited to, cusatuzumab, MDX-1411, vorsetuzumab and those described in WO2014158821 and WO2018152181, the contents of each of which are incorporated by reference herein in their entirety.

In one embodiment, a TFP that specifically binds human CD19 comprises a heavy chain variable region having the sequence of SEQ ID NO: 22 and a light chain variable region having the sequence of SEQ ID NO: 24. In some cases, the antigen binding domain is an anti-CD19 binding domain and the TFP comprises the sequence of SEQ ID NO: 22, SEQ ID NO: 24, or SEQ ID NO: 33. The antigen binding domain can comprise a variable domain comprising a light chain complementarity determining region 1 (LC CDR1), a LC CDR2, and a LC CDR3. The LC CDR1 can comprise the sequence of SEQ ID NO: 10, the LC CDR2 can comprise the sequence of SEQ ID NO: 12, and the LC CDR3 can comprise the sequence of SEQ ID NO: 14. The antigen binding domain can comprise a variable domain comprising a heavy chain complementarity determining region 1 (HC CDR1), a HC CDR2, and a HC CDR3. The HC CDR1 can comprise the sequence of SEQ ID NO: 16, the HC CDR2 can comprise the sequence of SEQ ID NO: 18, and the HC CDR3 can comprise the sequence of SEQ ID NO: 20.

In another embodiment, a TFP that specifically binds human MSLN comprises or consists of a VHH having a sequence of SEQ ID NO: 25 or SEQ ID NO: 26. In some cases, the antigen binding domain is an anti-MSLN binding domain and the TFP comprises the sequence of SEQ ID NO: 25, SEQ ID NO: 26, SEQ ID NO: 41, or SEQ ID NO: 34. The antigen binding domain can comprise a variable domain comprising a complementarity determining region (CDR) 1 of SEQ ID NO: 35, CDR2 of SEQ ID NO: 36, and CDR3 of SEQ ID NO: 37.

The antigen binding domain can comprise a variable domain comprising a CDR1 of SEQ ID NO: 38, CDR2 of SEQ ID NO: 39, and CDR3 of SEQ ID NO: 40. The antigen binding domain can comprise a variable domain comprising a CDR1 of SEQ ID NO: 41, CDR2 of SEQ ID NO: 42, and CDR3 of SEQ ID NO: 43.

In some embodiments, a TFP comprises an antigen binding domain that specifically binds CD70 (e.g., human CD70). Examples of the anti-CD70 binding domains are listed in Table 1. The TFP can comprise the sequence of SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, or SEQ ID NO: 80. wherein the antigen binding domain comprises a variable domain comprising a CDR1 of SEQ ID NO: 45, CDR2 of SEQ ID NO: 46, and CDR3 of SEQ ID NO: 47. The antigen binding domain can comprise a variable domain comprising a CDR1 of SEQ ID NO: 49, CDR2 of SEQ ID NO: 50, and CDR3 of SEQ ID NO: 51. The antigen binding domain can comprise a variable domain comprising a CDR1 of SEQ ID NO: 53, CDR2 of SEQ ID NO: 54, and CDR3 of SEQ ID NO: 55. The antigen binding domain can comprise a variable domain comprising a CDR1 of SEQ ID NO: 57, CDR2 of SEQ ID NO: 58, and CDR3 of SEQ ID NO: 59. The antigen binding domain can comprise a variable domain comprising a CDR1 of SEQ ID NO: 61, CDR2 of SEQ ID NO: 62, and CDR3 of SEQ ID NO: 63. The antigen binding domain can comprise a variable domain comprising a CDR1 of SEQ ID NO: 65, CDR2 of SEQ ID NO: 66, and CDR3 of SEQ ID NO: 67. The antigen binding domain can comprise a variable domain comprising a CDR1 of SEQ ID NO: 69, CDR2 of SEQ ID NO: 70, and CDR3 of SEQ ID NO: 71. The antigen binding domain can comprise a variable domain comprising a CDR1 of SEQ ID NO: 73, CDR2 of SEQ ID NO: 74, and CDR3 of SEQ ID NO: 75. The antigen binding domain can comprise a variable domain comprising a CDR1 of SEQ ID NO: 77, CDR2 of SEQ ID NO: 78, and CDR3 of SEQ ID NO: 79.

In one aspect, the present disclosure relates to an antigen binding domain comprising an antibody or antibody fragment, wherein the antibody binding domain specifically binds to a tumor-associated protein or fragment thereof, wherein the antibody or antibody fragment comprises a variable light chain and/or a variable heavy chain that includes an amino acid sequence provided herein. In certain aspects, the scFv is contiguous with and in the same reading frame as a leader sequence.

In one aspect, the anti-tumor-associated antigen binding domain is a fragment, e.g., a single chain variable fragment (scFv). In one aspect, the anti-TAA binding domain is a Fv, a Fab, a (Fab′)₂, or a bi-functional (e.g. bi-specific) hybrid antibody (e.g., Lanzavecchia et al., Eur. J. Immunol. 17, 105 (1987)). In one aspect, the antibodies and fragments thereof of the present disclosure binds a tumor-associated protein with wild-type or enhanced affinity. In another aspect, the anti-TAA binding domain comprises a single domain antibody (sdAb or V_{HH}).

Also provided herein are methods for obtaining an antibody antigen binding domain specific for a target antigen (e.g., CD19, BCMA, MUC16, MSLN, CD20, CD70, CD79B, HER2, PSMA or any target antigen described elsewhere herein for targets of fusion moiety binding domains), the method comprising providing by way of addition, deletion, substitution or insertion of one or more amino acids in the amino acid sequence of a V_{H} domain set out herein a V_{H} domain which is an amino acid sequence variant of the V_{H} domain, optionally combining the V_{H} domain thus provided with one or more V_{L} domains, and testing the V_{H} domain or V_{H}/V_{L} combination or combinations to identify a specific binding member or an antibody antigen binding domain specific for a target antigen of interest and optionally with one or more desired properties.

In some instances, V_{H} domains and scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking V_{H} and V_{L} regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intra-chain folding is prevented. Inter-chain folding is also required to bring the two variable regions together to form a functional epitope binding site. In some instances, the linker sequence comprises (G₄S)ₙ, wherein n=2 to 4. In some instances, the linker sequence comprises (G₄S)ₙ, wherein n=1 to 3. For examples of linker orientation and size see, e.g., Hollinger et al., 1993, Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, each of which is incorporated herein by reference.

An scFv can comprise a linker of about 10, 11, 12, 13, 14, 15 or greater than 15 residues between its V_{L} and V_{H} regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)ₙ, where n is a positive integer equal to or greater than 1. In one embodiment, the linker can be (Gly₄Ser)₄ or (Gly₄Ser)₃. Variation in the linker length may retain or enhance activity, giving rise to superior efficacy in activity studies. In some instances, the linker sequence comprises (G₄S)ₙ, wherein n=2 to 4. In some instances, the linker sequence comprises (G₄S)ₙ, wherein n=1 to 3.

### Stability and Mutations

The stability of a tumor associated antigen binding domain, e.g., scFv molecules (e.g., soluble scFv) can be evaluated in reference to the biophysical properties (e.g., thermal stability) of a conventional control scFv molecule or a full-length antibody. In one embodiment, the humanized or human scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a parent scFv in the described assays.

The improved thermal stability of the anti-TAA binding domain, e.g., scFv is subsequently conferred to the entire TAA-TFP construct, leading to improved therapeutic properties of the anti-TAA TFP construct. The thermal stability of the binding domain, e.g., scFv or sdAb, can be improved by at least about 2° C. or 3° C. as compared to a conventional antibody. In one embodiment, the binding domain, has a 1° C. improved thermal stability as compared to a conventional antibody. In another embodiment, the binding domain, has a 2° C. improved thermal stability as compared to a conventional antibody. In another embodiment, the scFv has a 4° C., 5° C., 6° C., 7° C., 8° C., 9° C., 10° C., 11° C., 12° C., 13° C., 14° C., or 15° C. improved thermal stability as compared to a conventional antibody. Comparisons can be made, for example, between the scFv molecules disclosed herein and scFv molecules or Fab fragments of an antibody from which the scFv V_{H} and V_{L} were derived. Thermal stability can be measured using methods known in the art. For example, in one embodiment, T_{M} can be measured. Methods for measuring T_{M} and other methods of determining protein stability are described in more detail below.

Mutations in antibody sequences (arising through humanization or direct mutagenesis of the soluble scFv) alter the stability of the antibody or fragment thereof and improve the overall stability of the antibody and the TFP construct. Stability of the humanized antibody or fragment thereof is compared against the murine antibody or fragment thereof using measurements such as T_{M}, temperature denaturation and temperature aggregation. In one embodiment, the binding domain, e.g., a scFv or sdAb, comprises at least one mutation arising from the humanization process such that the mutated scFv confers improved stability to the anti-TAA TFP construct. In another embodiment, the anti-TAA binding domain, e.g., scFv comprises at least 1, 2, 3, 4, 5, 6, 7, 8, 9, 10 mutations arising from the humanization process such that the mutated scFv confers improved stability to the TAA-TFP construct.

In one aspect, the antigen binding domain of the TFP comprises an amino acid sequence that is homologous to an antigen binding domain amino acid sequence described herein, and the antigen binding domain retains the desired functional properties of the anti-tumor-associated antigen antibody fragments described herein. In one specific aspect, the TFP composition of the present disclosure comprises an antibody fragment. In a further aspect, that antibody fragment comprises a scFv.

In various aspects, the antigen binding domain of the TFP is engineered by modifying one or more amino acids within one or both variable regions (e.g., V_{H} and/or V_{L}), for example within one or more CDR regions and/or within one or more framework regions. In one specific aspect, the TFP composition of the present disclosure comprises an antibody fragment. In a further aspect, that antibody fragment comprises a scFv.

It will be understood by one of ordinary skill in the art that the antibody or antibody fragment of the present disclosure may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990)J Mol. Biol. 215:403-410, respectively. Software for performing BLAST analyses is publicly available through the National Center for Biotechnology Information.

In one aspect, the present disclosure contemplates modifications of the starting antibody or fragment (e.g., scFv) amino acid sequence that generate functionally equivalent molecules. For example, the V_{H} or V_{L} of a binding domain, e.g., scFv, comprised in the TFP can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{H} or V_{L} framework region of the starting antibody fragment, e.g., scFv. The present disclosure contemplates modifications of the entire TFP construct, e.g., modifications in one or more amino acid sequences of the various domains of the TFP construct in order to generate functionally equivalent molecules. The TFP construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting TFP construct.

In another aspect, the TFP comprises an antigen-binding domain that comprises a ligand. In some embodiments, the ligand or fragment thereof is capable of binding to an antibody or fragment thereof. In some instances, the ligand binds to the polypeptide expressed on a surface of a cell. In some instances, the receptor or polypeptide expressed on a surface of a cell comprises a stress response receptor or polypeptide. In some instances, the receptor or polypeptide expressed on a surface of a cell is an MHC class I-related glycoprotein. In some instances, the MHC class I-related glycoprotein is selected from the group consisting of MICA (MHC class I polypeptide-related sequence A; UniProt ID: Q29983), MICB (MHC class I polypeptide-related sequence B; UniProt ID: Q29980), RAET1E (Retinoic acid early transcript 1E; UniProt ID: Q8TD07), RAET1G (UL-16 binding protein 5; UniProt ID: Q6H3X3), ULBP1 (UL16-binding protein 1; UniProt ID: Q9BZM6), ULBP2 (UL16-binding protein 2; UniProt ID: Q9BZM5), ULBP3 (UL16-binding protein 3; UniProt ID: Q9BZM4), ULBP4 (Retinoic acid early transcript 1E; UniProt ID: Q8TD07) and combinations thereof. In some instances, the antigen binding domain comprises a monomer, a dimer, a trimer, a tetramer, a pentamer, a hexamer, a heptamer, an octomer, a nonamer, or a decamer. In some instances, the antigen binding domain comprises a monomer or a dimer of the ligand or fragment thereof. In some instances, the ligand or fragment thereof is a monomer, a dimer, a trimer, a tetramer, a pentamer, a hexamer, a heptamer, an octomer, a nonamer, or a decamer. In some instances, the ligand or fragment thereof is a monomer or a dimer. In some instances, the antigen binding domain does not comprise an antibody or fragment thereof. In some instances, the antigen binding domain does not comprise a variable region. In some instances, the antigen binding domain does not comprise a CDR. In some instances, the ligand or fragment thereof is a Natural Killer Group 2D (NKG2D) ligand or a fragment thereof.

### Extracellular Domain

The extracellular domain may be derived either from a natural or from a recombinant source. Where the source is natural, the domain may be derived from any protein, but in particular a membrane-bound or transmembrane protein. In one aspect the extracellular domain is capable of associating with the transmembrane domain. An extracellular domain of particular use in this present disclosure may include at least the extracellular region(s) of e.g., the TCR gamma or delta subunits of the T cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, CD154, CD279.

In some embodiments, the TCR extracellular domain comprises an extracellular domain or portion thereof of a TCR delta chain or a TCR gamma chain. In some embodiments, the TCR extracellular domain comprises an IgC domain of a TCR delta chain or a TCR gamma chain.

In some embodiments, the extracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more consecutive amino acid residues of the extracellular domain of a TCR delta chain or a TCR gamma chain. In some embodiments, the extracellular domain comprises a sequence having at least about 50%, 55%, 60%, 65%, 70%, 75%, 80%, 85%, 90%, 95%, 98%, 99% or more sequence identity to a sequence encoding the extracellular domain of a TCR delta chain or a TCR gamma chain. In some embodiments, the extracellular domain comprises a sequence encoding the extracellular domain of a TCR delta chain or a TCR gamma chain having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

In some embodiments, the extracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more consecutive amino acid residues of an IgC domain of a TCR delta or a TCR gamma. In some embodiments, the extracellular domain comprises a sequence having at least about 50%, 55%, 60%, 65%, 70%, 75%, 80%, 85%, 90%, 95%, 98%, 99% or more sequence identity to a sequence encoding an IgC domain of a TCR delta or a TCR gamma. In some embodiments, the extracellular domain comprises a sequence encoding an IgC domain of TCR delta or TCR gamma having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

In some embodiments, the extracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more consecutive amino acid residues of the extracellular domain of a CD3 epsilon TCR subunit, a CD3 gamma TCR subunit, or a CD3 delta TCR subunit. In some embodiments, the extracellular domain comprises a sequence having at least about 50%, 55%, 60%, 65%, 70%, 75%, 80%, 85%, 90%, 95%, 98%, 99% or more sequence identity to a sequence encoding the extracellular domain of a CD3 epsilon TCR subunit, a CD3 gamma TCR subunit, or a CD3 delta TCR subunit. In some embodiments, the extracellular domain comprises a sequence encoding the extracellular domain of a CD3 epsilon TCR subunit, a CD3 gamma TCR subunit, or a CD3 delta TCR subunit having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

### Transmembrane Domain

In general, a TFP sequence contains an extracellular domain and a transmembrane domain encoded by a single genomic sequence. In alternative embodiments, a TFP can be designed to comprise a transmembrane domain that is heterologous to the extracellular domain of the TFP. A transmembrane domain can include one or more additional amino acids adjacent to the transmembrane region, e.g., one or more amino acid associated with the extracellular region of the protein from which the transmembrane was derived (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the intracellular region). In some cases, the transmembrane domain can include at least 30, 35, 40, 45, 50, 55, 60 or more amino acids of the extracellular region. In some cases, the transmembrane domain can include at least 30, 35, 40, 45, 50, 55, 60 or more amino acids of the intracellular region. In one aspect, the transmembrane domain is one that is associated with one of the other domains of the TFP is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP-T cell surface. In a different aspect the amino acid sequence of the transmembrane domain may be modified or substituted so as to minimize interactions with the binding domains of the native binding partner present in the same TFP.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFP has bound to a target. In some instances, the transmembrane domain comprises a transmembrane domain of a protein selected from the group consisting of a TCR gamma chain, a TCR delta chain, a CD3 zeta chain, a CD3 epsilon subunit, a CD3 gamma subunit, a CD3 delta subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some embodiments, the transmembrane domain comprises, or comprises at least 5, 6, 7, 8, 9, 10, 11, 12, 13, 14, 15, 16, 17, 18, 19, 20, 21, 22, 23, 24, 25, 26, 27, 28, 29, or 30 or more consecutive amino acid residues of the transmembrane domain of a TCR gamma chain, a TCR delta chain, a CD3 epsilon subunit, a CD3 gamma subunit, or a CD3 delta subunit. In some embodiments, the transmembrane domain comprises a sequence having at least about 50%, 55%, 60%, 65%, 70%, 75%, 80%, 85%, 90%, 95%, 98%, 99% or more sequence identity to a sequence encoding the transmembrane domain of a TCR gamma chain, a TCR delta chain, a CD3 epsilon subunit, a CD3 gamma subunit, or a CD3 delta subunit. In some embodiments, the transmembrane domain comprises a sequence encoding the transmembrane domain of a TCR gamma chain, a TCR delta chain, a CD3 epsilon subunit, a CD3 gamma subunit, or a CD3 delta subunit having a truncation of at least 1, 2, 3, 4, 5, 6, 7, 8, 9, or 10 or more amino acids at the N- or C-terminus or at both the N- and C-terminus.

In some instances, the transmembrane domain can be attached to the extracellular region of the TFP, e.g., the antigen binding domain of the TFP, via a hinge, e.g., a hinge from a human protein. For example, in one embodiment, the hinge can be a human immunoglobulin (Ig) hinge, e.g., an IgG4 hinge, or a CD8a hinge.

### Linkers

Optionally, a short oligo- or polypeptide linker, between 2 and 10 amino acids in length may form the linkage between the transmembrane domain and the cytoplasmic region of the TFP. In some cases, the linker may be at least about 5, 6, 7, 8, 9, 10, 11, 12, 13, 14, 15, 16, 17, 18, 19, 20, or more in length. A glycine-serine doublet provides a particularly suitable linker. For example, in one aspect, the linker comprises the amino acid sequence of GGGGSGGGGS (SEQ ID NO: 27) or a sequence (GGGGS)x wherein X is 1, 2, 3, 4, 5, 6, 7, 8, 9, or 10 or more. In some embodiments, X is 2. In some embodiments, X is 4. In some embodiments, the linker is encoded by a nucleotide sequence of GGTGGCGGAGGTTCTGGAGGTGGAGGTTCC (SEQ ID NO: 28).

### Cytoplasmic Domain

The cytoplasmic domain of the TFP can include an intracellular signaling domain, if the TFP contains CD3 gamma, delta, zeta, or epsilon polypeptides; TCR gamma and TCR delta subunits are generally lacking a signaling domain. An intracellular signaling domain is generally responsible for activation of at least one of the normal effector functions of the immune cell in which the TFP has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP of the present disclosure include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

In some embodiments, the intracellular domain comprises the intracellular domain of a TCR gamma chain, a TCR delta chain, a CD3 epsilon TCR subunit, a CD3 gamma TCR subunit, or a CD3 delta TCR subunit.

In some embodiments, the intracellular domain comprises, or comprises at least 1, 2, 3, 4, 5, 6, 7, 8, 9, 10, 11, 12, 13, 14, 15, 16, 17, 18, or 19 or more consecutive amino acid residues of the intracellular domain of a TCR gamma chain or a TCR delta chain. In some embodiments, the intracellular domain comprises a sequence having at least about 50%, 55%, 60%, 65%, 70%, 75%, 80%, 85%, 90%, 95%, 98%, 99% or more sequence identity to a sequence encoding the intracellular domain of a TCR gamma chain or a TCR delta chain. In some embodiments, the transmembrane domain comprises a sequence encoding the intracellular domain of a TCR gamma chain or a TCR delta chain having a truncation of at least 1, 2, 3, 4, 5, 6, 7, 8, 9, or 10 or more amino acids at the N- or C-terminus or at both the N- and C-terminus.

In some embodiments, the intracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 or more consecutive amino acid residues of the intracellular domain of a CD3 epsilon subunit, a CD3 gamma subunit, or a CD3 delta subunit. In some embodiments, the intracellular domain comprises a sequence having at least about 50%, 55%, 60%, 65%, 70%, 75%, 80%, 85%, 90%, 95%, 98%, 99% or more sequence identity to a sequence encoding the intracellular domain of a CD3 epsilon subunit, a CD3 gamma subunit, or a CD3 delta subunit. In some embodiments, the intracellular domain comprises a sequence encoding the intracellular domain of a CD3 epsilon subunit, a CD3 gamma subunit, or a CD3 delta subunit having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

It is known that signals generated through the TCR alone are insufficient for full activation of naive T cells and that a secondary and/or costimulatory signal is required. Thus, naïve T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the present disclosure include those of CD3 zeta, FcR gamma, FcR beta, CD3 gamma, CD3 delta, CD3 epsilon, CD5, CD22, CD79a, CD79b, and CD66d. In one embodiment, a TFP of the present disclosure comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP can comprise the CD3 zeta signaling domain by itself or it can be combined with any other desired intracellular signaling domain(s) useful in the context of a TFP of the present disclosure. For example, the intracellular signaling domain of the TFP can comprise a CD3 epsilon chain portion and a costimulatory signaling domain. The costimulatory signaling domain refers to a portion of the TFP comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human TFP-T cells in vitro and augments human T cell persistence and antitumor activity in vivo (Song et al., Blood. 2012; 119(3):696-706).

The intracellular signaling sequences within the cytoplasmic portion of the TFP of the present disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

In one embodiment, a glycine-serine doublet can be used as a suitable linker. In one embodiment, a single amino acid, e.g., an alanine, a glycine, can be used as a suitable linker.

In one aspect, the TFP-expressing cell described herein can further comprise a second TFP, e.g., a second TFP that includes a different antigen binding domain, e.g., to the same target (e.g., a first tumor associated antigen) or a different target (e.g., a second tumor associated antigen or cell recognition molecule). In one embodiment, when the TFP-expressing cell comprises two or more different TFPs, the antigen binding domains of the different TFPs can be such that the antigen binding domains do not interact with one another. For example, a cell expressing a first and second TFP can have an antigen binding domain of the first TFP, e.g., as a fragment, e.g., a scFv, that does not form an association with the antigen binding domain of the second TFP, e.g., the antigen binding domain of the second TFP is a V_{HH}.

In another aspect, the TFP-expressing cell described herein can further express another agent, e.g., an agent which enhances the activity of a modified T cell. For example, in one embodiment, the agent can be an agent which inhibits an inhibitory molecule. Inhibitory molecules, e.g., PD-L1, can, in some embodiments, decrease the ability of a modified T cell to mount an immune effector response. Examples of inhibitory molecules include PD-1, PD-L1, CTLA4, TIM3, LAG3, VISTA, BTLA, TIGIT, LAIRE CD160, 2B4 and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIRE TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2, have been shown to downregulate T cell activation upon binding to PD1 (Freeman et al., 2000 J Exp Med 192:1027-34; Latchman et al., 2001 Nat Immunol 2:261-8; Carter et al., 2002 Eur J Immunol 32:634-43). PD-L1 is abundant in human cancers (Dong et al., 2003 J Mol Med 81:281-7; Blank et al. 2005 Cancer Immunol. Immunother. 54:307-314; Konishi et al., 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1) that is fused to a transmembrane domain and optionally an intracellular signaling domain such as CD28, 41BB and CD3 zeta (also referred to herein as a PD1 TFP). In one another, the agent comprises the extracellular domain (ECD) and transmembrane domain of an inhibitory molecule, e.g., Programmed Death 1 (PD1) that is fused to an intracellular signaling domain such as CD28, 41BB and CD3 zeta (also referred to herein as a PD1 TFP). In one embodiment, the PD1 TFP, when used in combinations with an anti-TAA TFP described herein, improves the persistence of the T cell. In one embodiment, the TFP is a PD1 TFP comprising the extracellular domain of PD 1. Alternatively, provided are TFPs containing an antibody or antibody fragment such as a scFv that specifically binds to the Programmed Death-Ligand 1 (PD-L1) or Programmed Death-Ligand 2 (PD-L2).

In some embodiments, the agent is a cytokine. In some embodiments, the cytokine is IL-15. In some embodiments, IL-15 increases the persistence of the gamma delta T cells described herein.

In some embodiments, the recombinant nucleic acid encoding the TFP described herein comprises a first nucleic acid. The cell comprising the recombinant nucleic acid encoding the TFP can further comprise a second nucleic acid encoding IL-15. In some cases, the recombinant nucleic acid comprises the second nucleic acid. The IL-15 and the TFP can be expressed in frame from the recombinant nucleic acid. The IL-15 and the TFP, when expressed in frame from the recombinant nucleic acid, can be separated by a self-cleaving peptide, e.g., T2A. In some other cases, the IL-15 and the TFP can be expressed in two separate nucleic acid molecules. The expression of IL-15 can increase persistence of the cells.

In some embodiments, the cell comprising the recombinant nucleic acid encoding the TFP can comprise a first nucleic acid encoding an inhibitory molecule. The inhibitory molecule can comprise a first polypeptide comprising at least a portion of an inhibitory molecule, associated with a second polypeptide comprising a positive signal from an intracellular signaling domain. The inhibitory molecule can comprise the first polypeptide comprising at least a portion of PD-1 and the second polypeptide comprising a costimulatory domain and primary signaling domain. The second polypeptide can comprise at least a portion of CD28. The first polypeptide can comprise an extracellular domain and/or a transmembrane domain of PD-1. The transmembrane domain of PD-1 can be linked to an intracellular domain of CD28.

In some cases, the cell comprising the recombinant nucleic acid encoding the TFP can further comprise a nucleic acid sequence encoding IL-15 polypeptide or a fragment thereof. The IL-15 polypeptide can be secreted when expressed in a T cell. The IL-15 polypeptide can comprise a sequence of SEQ ID NO: 81. The nucleic acid sequence can further encode an IL-15 receptor (IL-15R) subunit or a fragment thereof. The IL-15R subunit can be IL-15R alpha (IL-15Rα). The nucleic acid sequence can encode a fusion protein comprising the IL-15 polypeptide linked to the IL-15Rα subunit. The fusion protein can comprise a sequence of SEQ ID NO: 84. The fusion protein can be expressed on cell surface when expressed. The fusion protein can be secreted when expressed. The cell can further comprise a first nucleic acid sequence encoding IL-15 receptor alpha (IL-15Rα) polypeptide or a fragment thereof. The first nucleic acid sequence can further encode PD-1 or a fragment thereof. The first nucleic acid sequence can further encode CD28 or a fragment thereof. The first nucleic acid sequence can encode a fusion protein comprising an extracellular domain and/or a transmembrane domain of PD-1 linked to an intracellular domain of CD28 linked to IL-15Rα. The fusion protein can comprise a sequence of SEQ ID NO: 85 or SEQ ID NO: 86. The cell can further comprise a second nucleic acid sequence encoding an IL-15 polypeptide or a fragment thereof. The IL-15 polypeptide or the fragment thereof can be secreted when expressed.

The TFP of the present invention may be used in multicistronic vectors or vectors expressing several proteins in the same transcriptional unit. Such vectors may use internal ribosomal entry sites (IRES). Since IRES are not functional in all hosts and do not allow for the stoichiometric expression of multiple protein, self-cleaving peptides may be used instead. For example, several viral peptides are cleaved during translation and allow for the expression of multiple proteins form a single transcriptional unit. Such peptides include 2A-peptides, or 2A-like sequences, from members of the Picornaviridae virus family. See for example Szymczak et al 2004, Nature Biotechnology; 22:589-594. In some embodiments, the recombinant nucleic acid described herein encodes the TFP in frame with the agent, with the two sequences separated by a self cleaving peptide, such as a 2A sequence, or a T2A sequence.

In another aspect, the present disclosure provides a population of TFP-expressing T cells, e.g., TFP-T cells. In some embodiments, the population of TFP-expressing T cells comprises a mixture of cells expressing different TFPs. For example, in one embodiment, the population of TFP-T cells can include a first cell expressing a TFP having a binding domain described herein, and a second cell expressing a TFP having a different anti-TAA binding domain, e.g., a binding domain described herein that differs from the binding domain in the TFP expressed by the first cell. As another example, the population of TFP-expressing cells can include a first cell expressing a TFP that includes an a first binding domain binding domain, e.g., as described herein, and a second cell expressing a TFP that includes an antigen binding domain to a target other than the binding domain of the first cell (e.g., another tumor-associated antigen).

In another aspect, the present disclosure provides a population of cells wherein at least one cell in the population expresses a TFP having a domain described herein, and a second cell expressing another agent, e.g., an agent which enhances the activity of a modified T cell. For example, in one embodiment, the agent can be an agent which inhibits an inhibitory molecule. Inhibitory molecules, e.g., can, in some embodiments, decrease the ability of a modified T cell to mount an immune effector response. Examples of inhibitory molecules include PD1, PD-L1, PD-L2, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein.

Disclosed herein are methods for producing in vitro transcribed RNA encoding TFPs. The present disclosure also includes a TFP encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In one aspect, the template includes sequences for the TFP.

In one aspect the anti-TAA TFP is encoded by a messenger RNA (mRNA) or circRNA. In one aspect the mRNA or circRNA encoding the anti-TAA TFP is introduced into a T cell for production of a TFP-T cell. In one embodiment, the in vitro transcribed RNA TFP can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a TFP of the present disclosure. In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3 ‘UTR sequences can decrease the stability of mRNA. Therefore, 3’ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts but do not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps can also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector®-II (Amaxa Biosystems, Cologne, Germany)), ECM 830 (BTX) (Harvard Instruments, Boston, Mass.) or the Gene Pulser® II (BioRad, Denver, Colo.), Multiporator® (Eppendorf, Hamburg Germany), cationic liposome mediated transfection using lipofection, polymer encapsulation, peptide mediated transfection, or biolistic particle delivery systems such as “gene guns” (see, for example, Nishikawa, et al., Hum Gene Ther., 12(8):861-70 (2001).

### Recombinant Nucleic Acids Encoding a TFP

Disclosed herein, in some embodiments, are recombinant nucleic acids comprising (a) a sequence encoding a T cell receptor (TCR) fusion protein (TFP) comprising (i) a TCR-integrating subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, or CD3 zeta; and (ii) an antibody comprising an antigen binding domain; wherein the TCR-integrating subunit and the antibody are operatively linked, and wherein the TFP functionally incorporates into a TCR complex when expressed in a T cell.

In some instances, the TCR-integrating subunit and the antibody domain or fragment thereof are operatively linked by a linker sequence. In some instances, the linker sequence comprises (G₄S)n, wherein n=1 to 5.

In some instances, the transmembrane domain is a TCR transmembrane domain from CD3 epsilon, CD3 gamma, CD3 delta, CD3 zeta, TCR gamma or TCR delta. In some instances, the intracellular domain is derived from only CD3 epsilon, only CD3 gamma, or only CD3 delta.

In some instances, the TCR-integrating subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR-integrating subunit comprises a transmembrane domain comprising a transmembrane domain of a protein selected from the group consisting of a TCR gamma chain, a TCR delta chain, a CD3 zeta chain TCR subunit, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR-integrating subunit comprises a TCR intracellular domain comprising a stimulatory domain of a protein selected from an intracellular signaling domain of CD3 epsilon, CD3 gamma or CD3 delta, CD3 zeta, or an amino acid sequence having at least one modification thereto.

In some instances, the TCR-integrating subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR-integrating subunit comprises an immunoreceptor tyrosine-based activation motif (ITAM) of a TCR-integrating subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of a CD3 zeta subunit, a CD3 epsilon subunit, a CD3 gamma subunit, and a CD3 delta subunit and replaces a different ITAM selected from the group consisting of a CD3 zeta subunit, a CD3 epsilon subunit, a CD3 gamma subunit, and a CD3 delta subunit.

In some instances, the at least one but not more than 20 modifications thereto comprise a modification of an amino acid that mediates cell signaling or a modification of an amino acid that is phosphorylated in response to a ligand binding to the TFP.

In some instances, the human or humanized antibody is an antibody fragment. In some instances, the antibody fragment is a scFv, a single domain antibody domain, a VH domain or a VL domain. In some instances, human or humanized antibody comprising an antigen binding domain is selected from a group consisting of an anti-CD19 binding domain, an anti-B-cell maturation antigen (BCMA) binding domain, an anti-mesothelin (MSLN) binding domain, an anti-PD-1 binding domain, anti-BAFF receptor binding domain, an anti-CD79B binding domain, an anti-CD70 binding domain, and an anti-HER2 binding domain, an anti-PMSA binding domain.

In some instances, the nucleic acid is selected from the group consisting of a DNA and an RNA. In some instances, the nucleic acid is an mRNA. In some instances, the nucleic acid is a circular RNA.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR gamma transmembrane domain. In some instances, the recombinant nucleic acid further comprises a sequence encoding a TCR delta transmembrane domain. In some instances, the recombinant nucleic acid further comprises a sequence encoding a TCR gamma transmembrane domain and a sequence encoding a TCR delta transmembrane domain.

Disclosed herein, in some embodiments, are recombinant nucleic acids comprising (a) a sequence encoding a T cell receptor (TCR) fusion protein (TFP) comprising (i) a TCR-integrating subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, or CD3 zeta, and (ii) a an antigen binding domain comprising ligand or a fragment thereof. In some embodiments, the ligand is capable of binding to an antibody or fragment thereof; wherein the TCR-integrating subunit and the ligand or fragment thereof are operatively linked, and wherein the TFP functionally incorporates into a TCR complex when expressed in a T cell. In some instances, the ligand is capable of binding an Fc domain of the antibody. In some instances, the ligand is capable of selectively binding an IgG1 antibody. In some instances, the ligand is capable of specifically binding an IgG1 antibody. In some instances, the antibody or fragment thereof binds to a cell surface antigen. In some instances, the antibody or fragment thereof binds to a cell surface antigen on the surface of a tumor cell. In some instances, the ligand comprises a monomer, a dimer, a trimer, a tetramer, a pentamer, a hexamer, a heptamer, an octomer, a nonamer, or a decamer. In some instances, the ligand does not comprise an antibody or fragment thereof. In some instances, the ligand comprises a CD16 polypeptide or fragment thereof. In some instances, the ligand comprises a CD16-binding polypeptide. In some instances, the ligand is human or humanized. In some instances, the recombinant nucleic acid further comprises a nucleic acid sequence encoding an antibody or fragment thereof capable of being bound by the ligand. In some instances, the antibody or fragment thereof is capable of being secreted from a cell.

In some instances, the TCR-integrating subunit and the antibody domain or the antigen binding domain or fragment thereof are operatively linked by a linker sequence. In some instances, the linker sequence comprises (G₄S)n, wherein n=1 to 5.

In some instances, the transmembrane domain is a TCR transmembrane domain from CD3 epsilon, CD3 gamma, CD3 delta, CD3 zeta, TCR gamma, or TCR delta. In some instances, the intracellular domain is derived from only CD3 epsilon, only CD3 gamma, only CD3 delta, only TCR gamma or only TCR delta.

In some instances, the TCR-integrating subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR-integrating subunit comprises a transmembrane domain comprising a transmembrane domain of a protein selected from the group consisting of a TCR gamma subunit, a TCR delta subunit, a CD3 epsilon subunit, a CD3 gamma subunit, a CD3 delta subunit, a CD3 zeta subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR-integrating subunit comprises a TCR intracellular domain comprising a stimulatory domain of a protein selected from an intracellular signaling domain of CD3 epsilon, CD3 gamma or CD3 delta, or an amino acid sequence having at least one modification thereto.

In some instances, the TCR-integrating subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR-integrating subunit comprises an immunoreceptor tyrosine-based activation motif (ITAM) of a TCR-integrating subunit that comprises an ITAM or portion thereof of a protein selected from the group consisting of CD3 zeta subunit, CD3 epsilon subunit, CD3 gamma subunit, CD3 delta subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta subunit, CD3 epsilon subunit, CD3 gamma subunit, and CD3 delta subunit and replaces a different ITAM selected from the group consisting of CD3 zeta subunit, CD3 epsilon subunit, CD3 gamma subunit, and CD3 delta subunit.

In some instances, the at least one but not more than 20 modifications thereto comprise a modification of an amino acid that mediates cell signaling or a modification of an amino acid that is phosphorylated in response to a ligand binding to the TFP.

In some instances, the human or humanized antibody is an antibody fragment. In some instances, the antibody fragment is a scFv, a single domain antibody domain, a VH domain or a VL domain. In some instances, human or humanized antibody comprising an antigen binding domain is selected from a group consisting of an anti-CD19 binding domain, an anti-B cell maturation antigen (BCMA) binding domain, an anti-mesothelin (MSLN) binding domain, an anti-MUC16 binding domain, an anti-PD-L1 binding domain, an anti-Nectin-4 binding domain, an anti-Trop-2 binding domain, an anti-GPC3 binding domain, or an anti-BAFFR binding domain, an anti-CD70 binding domain, an anti-HER2 binding domain, an anti-PMSA binding domain, and an anti-CD79B binding domain.

In some instances, the nucleic acid is selected from the group consisting of a DNA and an RNA. In some instances, the nucleic acid is an mRNA. In some instances, the nucleic acid is a circular RNA.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR gamma transmembrane domain. In some instances, the recombinant nucleic acid further comprises a sequence encoding a TCR delta transmembrane domain. In some instances, the recombinant nucleic acid further comprises a sequence encoding a TCR gamma transmembrane domain and a sequence encoding a TCR delta transmembrane domain. Alternatively, the recombinant nucleic acid comprises a sequence encoding a TCR gamma or TCR delta domain, e.g., a transmembrane domain.

Disclosed herein, in some embodiments, are recombinant nucleic acids comprising (a) a sequence encoding a T cell receptor (TCR) fusion protein (TFP) comprising (i) a TCR-integrating subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, or CD3 zeta; and (ii) an antigen binding domain comprising a ligand or a fragment thereof; wherein the TCR-integrating subunit and the antigen binding domain are operatively linked, and wherein the TFP functionally incorporates into a TCR complex when expressed in a T cell. In some instances, the ligand binds to the receptor of a cell. In some embodiments, the ligand or fragment thereof is capable of binding to an antibody or fragment thereof. In some embodiments, the ligand or fragment thereof binds to a receptor or polypeptide expressed on the surface of a cell. In some instances, the ligand binds to the polypeptide expressed on a surface of a cell. In some instances, the receptor or polypeptide expressed on a surface of a cell comprises a stress response receptor or polypeptide. In some instances, the receptor or polypeptide expressed on a surface of a cell is an MHC class I-related glycoprotein. In some instances, the MHC class I-related glycoprotein is selected from the group consisting of MICA (MHC class I polypeptide-related sequence A; UniProt ID: Q29983), MICB (MHC class I polypeptide-related sequence B; UniProt ID: Q29980), RAET1E (Retinoic acid early transcript 1E; UniProt ID: Q8TD07), RAET1G (UL-16 binding protein 5; UniProt ID: Q6H3X3), ULBP1 (UL16-binding protein 1; UniProt ID: Q9BZM6), ULBP2 (UL16-binding protein 2; UniProt ID: Q9BZM5), ULBP3 (UL16-binding protein 3; UniProt ID: Q9BZM4), ULBP4 (Retinoic acid early transcript 1E; UniProt ID: Q8TD07) and combinations thereof. In some instances, the antigen binding domain comprises a monomer, a dimer, a trimer, a tetramer, a pentamer, a hexamer, a heptamer, an octomer, a nonamer, or a decamer. In some instances, the antigen binding domain comprises a monomer or a dimer of the ligand or fragment thereof. In some instances, the ligand or fragment thereof is a monomer, a dimer, a trimer, a tetramer, a pentamer, a hexamer, a heptamer, an octomer, a nonamer, or a decamer. In some instances, the ligand or fragment thereof is a monomer or a dimer. In some instances, the antigen binding domain does not comprise an antibody or fragment thereof. In some instances, the antigen binding domain does not comprise a variable region. In some instances, the antigen binding domain does not comprise a CDR. In some instances, the ligand or fragment thereof is a Natural Killer Group 2D (NKG2D) ligand or a fragment thereof.

In some instances, the TCR-integrating subunit and the antibody domain or the antigen binding domain or fragment thereof are operatively linked by a linker sequence. In some instances, the linker sequence comprises (G₄S)n, wherein n=1 to 5.

In some instances, the transmembrane domain is a TCR transmembrane domain from CD3 epsilon, CD3 gamma, CD3 delta, CD3 zeta, TCR gamma or TCR delta. In some instances, the intracellular domain is derived from only CD3 epsilon, only CD3 gamma, or only CD3 delta.

In some instances, the TCR-integrating subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR-integrating subunit comprises a transmembrane domain comprising a transmembrane domain of a protein selected from the group consisting of a TCR gamma chain, a TCR delta chain, a CD3 zeta chain TCR subunit, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR-integrating subunit comprises a TCR intracellular domain comprising a stimulatory domain of a protein selected from an intracellular signaling domain of CD3 epsilon, CD3 gamma or CD3 delta, or an amino acid sequence having at least one modification thereto.

In some instances, the TCR-integrating subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR-integrating subunit comprises an immunoreceptor tyrosine-based activation motif (ITAM) of a TCR-integrating subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR-integrating subunit and replaces a different ITAM selected from the group consisting of CD3 zeta TCR subunit, CD3 epsilon TCR subunit, CD3 gamma TCR subunit, and CD3 delta TCR subunit.

In some instances, the at least one but not more than 20 modifications thereto comprise a modification of an amino acid that mediates cell signaling or a modification of an amino acid that is phosphorylated in response to a ligand binding to the TFP.

In some instances, the human or humanized antibody is an antibody fragment. In some instances, the antibody fragment is a scFv, a single domain antibody domain, a VH domain or a VL domain. In some instances, human or humanized antibody comprising an antigen binding domain is selected from a group consisting of an anti-CD19 binding domain, anti-B-cell maturation antigen (BCMA) binding domain, an anti-MUC16 binding domain, an anti-mesothelin (MSLN) binding domain, an anti-Nectin-4 binding domain, an anti-Trop-2 binding domain, an anti-GPC3 binding domain, or an anti-BAFFR binding domain, an anti-PD-L1 binding domain, an anti-CD70 binding domain, an anti-HER2 binding domain, an anti-PMSA binding domain, and an anti-CD79B binding domain.

In some instances, the nucleic acid is selected from the group consisting of a DNA and an RNA. In some instances, the nucleic acid is an mRNA. In some instances, the recombinant nucleic acid is a circular RNA.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR gamma transmembrane domain. In some instances, the recombinant nucleic acid further comprises a sequence encoding a TCR delta transmembrane domain. In some instances, the recombinant nucleic acid further comprises a sequence encoding a TCR gamma transmembrane domain and a sequence encoding a TCR delta transmembrane domain.

Further disclosed herein, in some embodiments, are vectors comprising the recombinant nucleic acid disclosed herein. In some instances, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some instances, the vector is an AAV6 vector. In some instances, the vector further comprises a promoter. In some instances, the vector is an in vitro transcribed vector.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In another embodiment, the vector comprising the nucleic acid encoding the desired TFP of the present disclosure is an adenoviral vector (A5/35). In another embodiment, the expression of nucleic acids encoding TFPs can be accomplished using of transposons such as sleeping beauty, crisper, CAS9, and zinc finger nucleases. See, e.g., June et al., 2009 Nature Reviews Immunology 9.10: 704-716, the entirety of which is incorporated herein by reference.

The expression constructs of the present disclosure may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties). In another embodiment, the present disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a TFP transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving TFP expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline-regulated promoter.

In order to assess the expression of a TFP polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, Molecular Cloning: A Laboratory Manual, volumes 1-4, Cold Spring Harbor Press, NY). A preferred method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In some embodiments, disclosed herein are circular RNAs, or circular RNA precursors encoding the TFP. To generate circRNAs that could subsequently be transferred into cells, in vitro production of circRNAs with autocatalytic-splicing introns can be programmed. A method for generating circRNA can involve in vitro transcription (IVT) of a precursor linear RNA template with specially designed primers. Methods for generating circRNAs are described, e.g., in Wesselhoeft et. al., Nat. Commun., 9:26-29, 2018.

circRNA can be introduced into target cells using any of a number of different methods, for instance, commercially available methods which include, but are not limited to, electroporation (Amaxa Nucleofector®-II (Amaxa Biosystems, Cologne, Germany)), ECM 830 (BTX) (Harvard Instruments, Boston, Mass.), Neon Transfection System (ThermoFisher), Cell squeezing (SQZ Biotechnologies) or the Gene Pulser® II (BioRad, Denver, Colo.), Multiporator® (Eppendorf, Hamburg Germany), polymer encapsulation, peptide mediated transfection, or biolistic particle delivery systems such as “gene guns” (see, for example, Nishikawa, et al. Hum Gene Ther., 12(8):861-70 (2001).

In the case where a non-viral delivery system is utilized, including circRNA, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and western blots) or by assays described herein to identify agents falling within the scope of the present disclosure.

The present disclosure further provides a vector comprising a TFP encoding nucleic acid molecule. In one aspect, a TFP vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the TFP construct in mammalian γδ T cells. In one aspect, the mammalian γδ T cell is a human γδ T cell.

### Modified γδ T Cells

Disclosed herein, in some embodiments, are modified γδ T cells comprising the recombinant nucleic acid disclosed herein, or the vectors disclosed herein. The modified γδ T cell may comprise a functional disruption of an endogenous TCR. Also disclosed herein, in some embodiments, are modified γδ T cells comprising the sequence of the nucleic acid disclosed herein encoding the TFP or a TFP encoded by the sequence of the nucleic acid disclosed herein. Further disclosed herein, in some embodiments, are modified allogenic γδ T cells comprising the sequence encoding the TFP disclosed herein or a TFP encoded by the sequence of the nucleic acid disclosed herein. In some embodiments, the γδ T cells are Vδ1+Vδ2−γδ T cells. In some embodiments, the γδ T cells are Vδ1− Vδ2+γδ T cells. In some embodiments, the γδ T cells are Vδ1−Vδ2−γδ T cells.

### Sources of γδ T Cells

Prior to expansion and genetic modification, a source of γδ T cells is obtained from a subject. The term “subject” is intended to include living organisms in which an immune response can be elicited (e.g., mammals). Examples of subjects include humans, dogs, cats, mice, rats, and transgenic species thereof.

In some aspects, the γδ T cells are obtained from a bank of umbilical cord blood, peripheral blood, human embryonic stem cells, or induced pluripotent stem cells, for example. Suitable doses for a therapeutic effect would be at least 10⁵ or between about 10⁵ and about 10¹⁰ cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass.

An effective amount or sufficient number of the isolated, oligoclonal γδ T cells is present in the composition and introduced into the subject such that long-term, specific, anti-tumor responses are established to reduce the size of a tumor or eliminate tumor growth or regrowth than would otherwise result in the absence of such treatment. Desirably, the amount of oligoclonal γδ T cells introduced into the subject causes a 10%, 20%, 30%, 40%, 50%, 60%, 70%, 80%, 90%, 95%, 98%, or 100% decrease in tumor size when compared to otherwise same conditions wherein the oligoclonal γδ T cells are not present.

Accordingly, the amount of oligoclonal γδ T cells administered should take into account the route of administration and should be such that a sufficient number of the oligoclonal γδ T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of oligoclonal γδ T cells desirably should be sufficient to provide in the subject being treated at least from about 1×10⁶ to about 1×10⁹ oligoclonal γδ T cells, even more desirably, from about 1×10⁷ to about 5×10⁸ oligoclonal γδ T cells, although any suitable amount can be utilized either above, e.g., greater than 5×10⁸ cells, or below, e.g., less than 1×10⁷ cells. The dosing schedule can be based on well-established cell-based therapies (see, e.g., Topalian and Rosenberg, 1987; U.S. Pat. No. 4,690,915), or an alternate continuous infusion strategy can be employed. These values provide general guidance of the range of oligoclonal γδ T cells to be used upon optimizing the method of the present invention for practice of the invention.

Enrichment of a T cell population by negative selection can be accomplished with a combination of antibodies directed to surface markers not expressed in the negatively selected cells. One method is cell sorting and/or selection via negative magnetic immunoadherence or flow cytometry that uses a cocktail of monoclonal antibodies directed to cell surface markers absent on the cells negatively selected. For example, to enrich for γδ T cells by negative selection, a monoclonal antibody cocktail typically includes antibodies to CD14, CD4, CD20, CD11b, CD16, HLA-DR, TCRαβ and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection. In some embodiments,

In one embodiment, a T cell population can be selected that expresses one or more of IFN-γ TNF-α, IL-17A, IL-2, IL-3, IL-4, GM-CSF, IL-10, IL-13, granzyme B, and perform, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 million cells/mL is used. In one aspect, a concentration of 1 million cells/mL is used. In a further aspect, greater than 100 million cells/mL is used. In a further aspect, a concentration of cells of 10, 15, 20, 25, 30, 35, 40, 45, or 50 million cells/mL is used. In yet one aspect, a concentration of cells from 75, 80, 85, 90, 95, or 100 million cells/mL is used. In further aspects, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/mL. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. In certain aspects, cryopreserved cells are thawed and washed as described herein and allowed to rest for one hour at room temperature prior to activation using the methods of the present disclosure.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 7,572,631. In some embodiments, PBMCs are activated and expanded prior to negative selection to obtain γδ T cells. In other embodiments, γδ T cells are obtained by negative selection and are then activated and expanded.

In certain aspects, the invention includes a method of making and/or expanding oligoclonal γδ T cells that comprises culturing the cells with artificial antigen presenting cells (APCs). In certain aspects, the γδ T cells are primary human γδ T cells, such as γδ T cells derived from human peripheral blood mononuclear cells (PBMC), PBMC collected after stimulation with G-CSF, bone marrow, or umbilical cord blood. The cells may be propagated for days, weeks, or months ex vivo as a bulk population in co-culture with aAPCs. Co-cultures may be initiated with 10³, 10⁴, 10⁵, 10⁶, 10⁷, or 10⁸ γδ T cells, or any number derivable therein, and 10³, 10⁴, 10⁵, 10⁶, 10⁷, 10⁸, or 10⁹ aAPC, or any number derivable therein. It is preferable that the co-cultures be initiated with a ratio of γδ T cells to aAPC of 1 to 2. In some embodiments, the cells are co-cultured with aAPCs and IL-2, and/or IL-21.

In some embodiments, the γδ T cells may be activated and/or expanded by stimulation with IL-2, IL-7, IL-15, or other cytokines that bind the common gamma-chain (e.g., IL-12, IL-21, and others). The activation and/or expansion of γδ T cells may be stimulated with 10, 20, 30, 40, 50, 60, 70, 80, 90, 100, 200, 300, 400, 500, 600, 700, 800, 900, or 100 U/mL of IL-2; preferably the activation and/or expansion is stimulated with 300 U/mL of IL-2. The activation and/or expansion of γδ T cells may be stimulated with 1, 2, 3, 4, 5, 6, 7, 8, 9, 10, 11, 12, 13, 14, 15, 16, 17, 18, 19, 20, 30, 40, 50, or 100 ng/mL of IL-7; preferably the activation and/or expansion is stimulated with 12.5 ng/mL IL-7. The activation and/or expansion of γδ T cells may be stimulated with 1, 2, 3, 4, 5, 6, 7, 8, 9, 10, 11, 12, 13, 14, 15, 16, 17, 18, 19, 20, 30, 40, 50, or 100 ng/mL of IL-15; preferably the activation and/or expansion is stimulated with 12.5 ng/mL IL-15. Preferably the activation and/or expansion is stimulated with 10-20 ng/mL IL-7 and 10-20 ng/mL IL-15, e.g., 12.5 ng/mL IL-7 and 12.5 ng/mL IL-15. Said stimulations may occur 1, 2, 3, 4, 5, 6, or 7 times per week, preferably every 2 days. In a further aspect, the expanded γδ T cells may be cryopreserved.

In some embodiments, the T cells of the present disclosure may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of γδ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

In some embodiments, γδ T cells are activated by stimulation with an anti-CD3 antibody and an anti-CD28 antibody in combination with cytokines that bind the common gamma-chain (e.g., IL-2, IL-7, IL-12, IL-15, IL-21, and others). In particular embodiments, γδ T cells that have been isolated from PBMC by negative selection are activated with IL-2 in the presence of bead-bound anti-CD3 and anti-CD28 antibodies (Dynabeads). In some embodiments, γδ T cells activated with IL-2 in the presence of bead-bound anti-CD3 and anti-CD28 antibodies are transduced 3, 4, 5, or 6 days after activation. In some embodiments, γδ T cells that are activated with IL-2 in the presence of bead-bound anti-CD3 and anti-CD28 antibodies are expanded with IL-2 in the absence of bead-bound anti-CD3 and anti-CD28 antibodies. In particular embodiments, γδ T cells are activated with IL-7 and IL-15 in the presence of bead-bound anti-CD3 and anti-CD28 antibodies (TransAct beads). In some embodiments, γδ T cells activated with IL-7 and IL-15 in the presence of bead-bound anti-CD3 and anti-CD28 antibodies are transduced 1 or 2 days after activation. In some embodiments, γδ T cells that are activated with IL-7 and IL-15 in the presence of bead-bound anti-CD3 and anti-CD28 antibodies are expanded with IL-7 and IL-15 in the absence of bead-bound anti-CD3 and anti-CD28 antibodies. In some embodiments, cells are subcultured every 1, 2, 3, 4 or 5 days. In some embodiments, cells are cultured at a concentration of 10⁴, 10⁵, 10⁶, or 10⁷ γδ T cells

The expansion of γδ T cells may be stimulated with zoledronic acid (Zometa), alendronic acid (Fosamax) or other related bisphosphonate drugs at concentrations of 0.1, 0.25, 0.5, 1.0, 2.0, 3.0, 4.0, 5.0, 7.5, 10, or 100 μM in the presence of feeder cells (irradiated cancer cells, PBMCs, artificial antigen presenting cells). The expansion of γδ T cells may be stimulated with isopentyl pyrophosphate (IPP), (E)-4-Hydroxy-3-methyl-but-2-enyl pyrophosphate (HMBPP or HMB-PP) or other structurally related compounds at concentrations of 0.1, 0.25, 0.5, 1.0, 2.0, 3.0, 4.0, 5.0, 7.5, 10, or 100 μM in the presence of feeder cells (irradiated cancer cells, PBMCs, artificial antigen presenting cells). In some embodiments, the expansion of γδ T cells may be stimulated with synthetic phosphoantigens (e.g., bromohydrin pyrophosphate; BrHPP), 2M3B1 PP, or 2-methyl-3-butenyl-1-pyrophosphate in the presence of IL-2 for one-to-two weeks. In some embodiments, the expansion of γδ T cells may be stimulated with immobilized anti-TCRyd (e.g., pan TCRY6) in the presence of IL-2, e.g., for approximately 14 days. In some embodiments, the expansion of γδ T cells may be stimulated with culture of immobilized anti-CD3 antibodies (e.g., OKT3) in the presence of IL-2. In some embodiments, the aforementioned culture is maintained for about seven days prior to subculture in soluble anti-CD3, and IL-2.

In some cases, provided herein are methods of producing a plurality of cells comprising the cell (e.g., modified γδ T cell) described herein, which cell comprises the recombinant nucleic acid encoding the TFP. For example, the method can comprise activating the γδ T cells isolated from PBMCs obtained from a donor. Activation can comprise contacting the γδ T cells with one or more immunomodulatory agents. In some cases, activation can further comprise contacting the γδ T cells with one or more cytokines. Next, the γδ T cells can be transduced with the viral vector described herein (e.g., the vector comprising the recombinant nucleic acid sequence encoding the TFP). Next, the γδ T cells can be expanded in the presence of the one or more cytokines. The method can further comprise obtaining the PBMCs from a donor. The method can further comprise isolating γδ T cells from the PBMCs. The γδ T cells can be obtained by negative selection. The γδ T cells can be obtained by positive selection.

The one or more immunomodulatory agents can comprise concanavalin (e.g., concanavalin A), an anti-CD3 antibody, an anti-CD28 antibody, an anti-TCR delta antibody, an anti-TCR gamma antibody, or a pan Gamma delta TCR antibody. The anti-TCR delta antibody can be an anti-TCR delta1 antibody. The anti-TCR gamma antibody can be an anti-TCR gamma9 antibody. The anti-TCR delta antibody, the anti-TCR gamma antibody, the pan Gamma delta TCR antibody can be plate bound. The one or more immunomodulatory agents can comprise anti-CD3 and anti-CD28 antibodies, e.g., human anti-CD3 and anti-CD28 antibodies. The anti-CD3 and the anti-CD28 antibodies can be bead bound. The anti-CD3 and the anti-CD28 antibodies can be suspended in a matrix. The one or more cytokines can be IL-2, IL-4, IL-7 or IL-15. The one or more cytokines can be IL-7 and IL-15. The one or more cytokines can be IL-2, IL-7, and IL-15. The one or more cytokines can be IL-2 and IL-4. The one or more immunomodulatory agents can further comprise a retronectin. The retronectin can be plate bound.

The one or more immunomodulatory agents can comprise anti-CD3 and anti-CD28 antibodies, e.g., human anti-CD3 and anti-CD28 antibodies. In some cases, the one or more cytokines can be IL-7 and IL-15.

The one or more immunomodulatory agents can comprise anti-CD3 and anti-CD28 antibodies, e.g., human anti-CD3 and anti-CD28 antibodies. In some cases, the one or more cytokines can be IL-2, IL-7, and IL-15.

The one or more immunomodulatory agents can comprise a retronectin and anti-CD3 and anti-CD28 antibodies, e.g., human anti-CD3 and anti-CD28 antibodies. In some cases, the one or more cytokines can be IL-7 and IL-15.

The anti-CD3 and the anti-CD28 antibodies can be bead bound. The anti-CD3 and the anti-CD28 antibodies can be suspended in a matrix.

The one or more immunomodulatory agents can comprise the concanavalin and the one or more cytokines can be IL-2 and IL-4. The one or more immunomodulatory agents can comprise an anti-TCR delta1 antibody and the one or more cytokines can be IL-2, IL-7, and IL-15. The one or more immunomodulatory agents can comprise an anti-TCR gamma9 antibody and the one or more cytokines can be IL-2, IL-7, and IL-15. The one or more immunomodulatory agents can comprise a retronectin and the pan Gamma delta TCR antibody and the one or more cytokines can be IL-2, IL-7, and IL-15.

The one or more immunomodulatory agents can comprise a retronectin and an anti-TCR gamma9 antibody and the one or more cytokines can be IL-2, IL-7, and IL-15.

The cells can be activated in the presence of the immunomodulatory agents for 2, 3, 4, or 5 days and then expanded in the absence of the immunomodulatory agents. In some cases, the cells can be activated in the presence of the immunomodulatory agents for 5 or more days before being expanded in the absence of the immunomodulatory agents.

The final concentration of the one or more cytokines for expanding the cells in the mixture or culture can vary. In some cases, the final concentration of the cytokine (e.g., IL-2, IL-4, IL-7, or IL-15) can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more ng/mL. In some cases, the final concentration of the cytokine can be at least about 50, 100, 150, 200 or more ng/mL. For example, the final concentration of IL-7 can be about 1, 2, 3, 4, 5, 6, 7, 8, 9, 10, 11, 12, 13, 14, 15, 16, 17, 18, 19, 20, or more ng/mL. For another example, the final concentration of IL-15 can be about 1, 2, 3, 4, 5, 6, 7, 8, 9, 10, 11, 12, 13, 14, 15, 16, 17, 18, 19, 20, or more ng/mL. For another example, the final concentration of IL-4 can be about 1, 2, 3, 4, 5, 6, 7, 8, 9, 10, 11, 12, 13, 14, 15, 16, 17, 18, 19, 20, or more ng/mL. For another example, the final concentration of IL-2 can be about 1, 2, 3, 4, 5, 6, 7, 8, 9, 10, 11, 12, 13, 14, 15, 16, 17, 18, 19, 20, or more ng/mL. In some cases, the final concentration of IL-2 can be at least about 50, 60, 70, 80, 90, 100, 150, 200 or more units/mL.

The final concentration of the immunomodulatory agents such as concanavalin can be at least about 50, 100, 150, 200, 400, 500, 800, 1,000, 1,500, 2,000, 2,500 or more ng/mL. For example, the final concentration of concanavalin A can be about 1,000 ng/mL.

The cells described herein can be activated by contacting the γδ T cells with a ligand that binds the antigen binding domain of the TFP. In some embodiments, the ligand is the antigen that is bound by the antigen binding domain. In some embodiments, the antigen is a TAA. In some embodiments, the antigen is CD19, BCMA, MSLN, MUC16, HER2, PSMA, CD70, CD79B, PD-L1, Nectin-4, Trop-2, GPC3, or BAFF receptor. In some embodiments, the ligand is expressed on the surface of an antigen-presenting cell (APC). In some embodiments, the γδ T cells are co-cultured with the APCs expressing the ligand on their cell surface. In some cases, the method can comprise activating the γδ T cells by contacting the γδ T cells with an APC in the presence or absence of the one or more immunomodulatory agents. The APC can express a ligand of the antigen binding domain of the TFP. The cells comprising the TFP can be selectively expanded by contacting the APC. In some cases, the γδ T cells can be contacted with the APC after removal of the one or more immunomodulatory agents. The γδ T cells can be contacted with the APC in the absence of the one or more immunomodulatory agents. The APC can be a professional, non-professional or artificial APC.

Further, in addition to Vδ1+ and Vδ2+ TCR-specific markers, other phenotypic markers vary significantly, but in large part, reproducibly during the course of the cell expansion process. Thus, such reproducibility enables the ability to tailor an activated T cell product for specific purposes.

Once a TFP described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a TFP described herein are described in further detail below.

Western blot analysis of TFP expression in primary γδ T cells can be used to detect the presence of monomers and dimers (see, e.g., Milone et al., Molecular Therapy 17(8): 1453-1464 (2009)). Very briefly, γδ T cells expressing the TFPs are expanded in vitro for more than 10 days followed by lysis and SDS-PAGE under reducing conditions. TFPs are detected by western blotting using an antibody to a TCR chain. The same T cell subsets are used for SDS-PAGE analysis under non-reducing conditions to permit evaluation of covalent dimer formation.

In vitro expansion of TFP⁺ T cells following antigen stimulation can be measured by flow cytometry. For example, a mixture of Vδ1+ and Vδ2+γδ T cells are stimulated with alphaCD3/alphaCD28 and APCs followed by transduction with lentiviral vectors expressing GFP under the control of the promoters to be analyzed. Exemplary promoters include the CMV IE gene, EF-1alpha, ubiquitin C, or phosphoglycerokinase (PGK) promoters. GFP fluorescence is evaluated on day 6 of culture in the CD4+ and/or CD8+ T cell subsets by flow cytometry (see, e.g., Milone et al., Molecular Therapy 17(8): 1453-1464 (2009)).

Alternatively, a mixture of Vδ1+ and Vδ2+ γδ T cells are stimulated with anti-CD3/anti-CD28 coated magnetic beads on day 0, and transduced with TFP on day 1 using a bicistronic lentiviral vector expressing TFP along with eGFP using a 2A ribosomal skipping sequence. Cultures are re-stimulated with either CD19+K562 cells (K562-CD19), MSLN+ MSTO cells (MSTO-MSLN), wild-type K562 cells (K562 wild type), wild-type MSTO cells (MSTO) or K562 cells expressing hCD32 and 4-1BBL in the presence of antiCD3 and anti-CD28 antibody (K562-BBL-3/28) following washing. Exogenous IL-2, or IL-7 plus IL-15, is added to the cultures every other day at 100 IU/mL. GFP+ T cells are enumerated by flow cytometry using bead-based counting (see, e.g., Milone et al., Molecular Therapy 17(8): 1453-1464 (2009)).

Sustained TFP+ T cell expansion in the absence of re-stimulation can also be measured (see, e.g., Milone et al., Molecular Therapy 17(8): 1453-1464 (2009)). Briefly, mean T cell volume (fl) is measured on day 8 of culture using a Coulter Multisizer III particle counter following stimulation with alphaCD3/alphaCD28 coated magnetic beads on day 0, and transduction with the indicated TFP on day 1.

Animal models can also be used to measure a TFP-T activity. For example, xenograft model using human CD19-specific TFP+ T cells to treat a primary human pre-B ALL in immunodeficient mice can be used (see, e.g., Milone et al., Molecular Therapy 17(8): 1453-1464 (2009)). Very briefly, after establishment of ALL, mice are randomized as to treatment groups. Different numbers of engineered T cells are coinjected at a 1:1 ratio into NOD/SCID/γ−/− mice bearing B-ALL. The number of copies of each vector in spleen DNA from mice is evaluated at various times following T cell injection. Animals are assessed for leukemia at weekly intervals. Peripheral blood CD19+ B-ALL blast cell counts are measured in mice that are injected with alphaCD19-zeta TFP+ T cells or mock-transduced T cells. Survival curves for the groups are compared using the log-rank test. In addition, absolute peripheral blood Vδ1+ and Vδ2+γδ T cell counts 4 weeks following T cell injection in NOD/SCID/γ−/− mice can also be analyzed. Mice are injected with leukemic cells and 3 weeks later are injected with T cells engineered to express TFP by a bicistronic lentiviral vector that encodes the TFP. T cells are normalized to ˜40% input TFP+ T cells. Animals are assessed for leukemia at 1-week intervals. Survival curves for the TFP+ T cell groups are compared using the log-rank test.

Dose dependent TFP treatment response can be evaluated (see, e.g., Milone et al., Molecular Therapy 17(8): 1453-1464 (2009)). For example, peripheral blood is obtained 13 days and every week thereafter establishing leukemia in mice injected on day 6 with TFP T cells, an equivalent number of mock-transduced T cells, or no T cells. Mice from each group are randomly bled for determination of peripheral blood CD19+ ALL blast counts.

Cell proliferation and cytokine production can also be measured, see, e.g., at Milone et al., Molecular Therapy 17(8): 1453-1464 (2009). Briefly, assessment of TFP-mediated proliferation can be performed in microtiter plates by mixing washed T cells with K562 cells expressing CD19 (K19) or MSTO cells expressing MSLN at 10:1, 3:1, 2:1, 1:1, 1:3, and 1:10 ratio. Anti-CD3 (clone OKT3) and anti-CD28 (clone 9.3) monoclonal antibodies are added to cultures with K562 or MSTO cells to serve as a positive control for stimulating T cell proliferation since these signals support long-term γδ T cell expansion ex vivo. T cells are enumerated in cultures using CountBright™ fluorescent beads (Invitrogen) and flow cytometry as described by the manufacturer. TFP+ T cells are identified by GFP expression using T cells that are engineered with eGFP-2A linked TFP-expressing lentiviral vectors. For TFP+ T cells not expressing GFP, the TFP+ T cells are detected with biotinylated recombinant CD19 protein and a secondary avidin-PE conjugate. Vδ1+ and Vδ2+ expression on T cells are also simultaneously detected with specific monoclonal antibodies (BD Biosciences). Cytokine measurements are performed on supernatants collected 24 hours following re-stimulation using the human Th1/Th2 cytokine cytometric bead array kit (BD Biosciences) according the manufacturer's instructions. Fluorescence is assessed using a Fortessa flow cytometer (BD Biosciences), and data are analyzed according to the manufacturer's instructions.

Cytotoxicity can be assessed by a standard ⁵¹Cr-release assay (see, e.g., Milone et al., Molecular Therapy 17(8): 1453-1464 (2009)). Target cells (K562 lines and primary pro-B-ALL cells) are loaded with ⁵¹Cr (as NaCrO₄, New England Nuclear) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of Triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average ⁵¹Cr released for each experimental condition.

Imaging technologies can be used to evaluate specific trafficking and proliferation of TFPs in tumor-bearing animal models. Such assays have been described, e.g., in Barrett et al., Human Gene Therapy 22:1575-1586 (2011). NOD/SCID/γc−/− (NSG) mice are injected IV with Nalm-6 cells (ATCC® CRL-3273™) followed 7 days later with T cells 4 hour after electroporation with the TFP constructs. The T cells are stably transfected with a lentiviral construct to express firefly luciferase, and mice are imaged for bioluminescence. Alternatively, therapeutic efficacy and specificity of a single injection of TFP+ T cells in Nalm-6 xenograft model can be measured as the following: NSG mice are injected with Nalm-6 transduced to stably express firefly luciferase, followed by a single tail-vein injection of T cells electroporated with CD19 TFP 7 days later. Animals are imaged at various time points post injection. For example, photon-density heat maps of firefly luciferase positive leukemia in representative mice at day 5 (2 days before treatment) and day 8 (24 hours post TFP+ PBLs) can be generated.

Other assays, including those described in the Example section herein as well as those that are known in the art can also be used to evaluate the TFP constructs disclosed herein.

### Pharmaceutical Compositions

Disclosed herein, in some embodiments, are pharmaceutical compositions comprising: (a) the modified T cells of the disclosure; and (b) a pharmaceutically acceptable carrier. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present disclosure, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present disclosure are administered to a patient by intradermal or subcutaneous injection. In one aspect, the T cell compositions of the present disclosure are administered by i.v. injection. The compositions of T cells may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more TFP constructs of the present disclosure may be introduced, thereby creating a modified T cell of the present disclosure. Subjects in need thereof may subsequently undergo standard treatment with high dose chemotherapy followed by peripheral blood stem cell transplantation. In certain aspects, following or concurrent with the transplant, subjects receive an infusion of the expanded modified T cells of the present disclosure. In an additional aspect, expanded cells are administered before or following surgery.

As is described herein, in some embodiments, the subject does not undergo leukapheresis, and is administered allogeneic cells obtained from a donor subject, which are modified to express the TFP using the methods described herein.

The dosage of the above treatments to be administered to a patient will vary with the precise nature of the condition being treated and the recipient of the treatment. The scaling of dosages for human administration can be performed according to art-accepted practices. The dose for alemtuzumab, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the TFP is introduced into T cells, e.g., using in vitro transcription, and the subject (e.g., human) receives an initial administration of TFP T cells of the present disclosure, and one or more subsequent administrations of the TFP T cells of the present disclosure, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the TFP T cells of the present disclosure are administered to the subject (e.g., human) per week, e.g., 2, 3, or 4 administrations of the TFP T cells of the present disclosure are administered per week. In one embodiment, the subject (e.g., human subject) receives more than one administration of the TFP T cells per week (e.g., 2, 3 or 4 administrations per week) (also referred to herein as a cycle), followed by a week of no TFP T cells administrations, and then one or more additional administration of the TFP T cells (e.g., more than one administration of the TFP T cells per week) is administered to the subject. In another embodiment, the subject (e.g., human subject) receives more than one cycle of TFP T cells, and the time between each cycle is less than 10, 9, 8, 7, 6, 5, 4, or 3 days. In one embodiment, the TFP T cells are administered every other day for 3 administrations per week. In one embodiment, the TFP T cells of the present disclosure are administered for at least two, three, four, five, six, seven, eight or more weeks.

In one aspect, TFP T cells are generated using lentiviral viral vectors, such as lentivirus. In another aspect, TFP cells are generated using circRNA. In another aspect, TFP cells are generated using retroviral vectors, such as a retrovirus. TFP-T cells generated that way will have stable TFP expression.

In one aspect, TFP T cells transiently express TFP vectors for 4, 5, 6, 7, 8, 9, 10, 11, 12, 13, 14, 15 days after transduction. Transient expression of TFPs can be effected by RNA TFP vector delivery. In one aspect, the TFP RNA is transduced into the T cell by electroporation.

A potential issue that can arise in patients being treated using transiently expressing TFP T cells (particularly with murine scFv bearing TFP T cells) is anaphylaxis after multiple treatments.

Without being bound by this theory, it is believed that such an anaphylactic response might be caused by a patient developing humoral anti-TFP response, i.e., anti-TFP antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-TFP antibody response during the course of transient TFP therapy (such as those generated by RNA transductions), TFP T cell infusion breaks should not last more than ten to fourteen days.

### Methods of Treatment

Disclosed herein, in some embodiments, are methods of treating cancer in a subject in need thereof, the method comprising administering to the subject a therapeutically effective amount of the pharmaceutical compositions disclosed herein. Further disclosed herein, in some embodiments, are methods of treating cancer in a subject in need thereof, the method comprising administering to the subject a pharmaceutical composition comprising (a) a modified T cell produced according to the methods disclosed herein; and (b) a pharmaceutically acceptable carrier.

In some instances, the modified T cell is an allogeneic T cell. In some instances, less cytokines are released in the subject compared a subject administered an effective amount of an unmodified control T cell. In some instances, less cytokines are released in the subject compared a subject administered an effective amount of a modified T cell comprising the recombinant nucleic acid disclosed herein, or the vector disclosed herein.

In some instances, the method comprises administering the pharmaceutical composition in combination with an agent that increases the efficacy of the pharmaceutical composition. In some instances, the method comprises administering the pharmaceutical composition in combination with an agent that ameliorates one or more side effects associated with the pharmaceutical composition.

In some instances, the cancer is a solid cancer, a lymphoma or a leukemia. In some instances, the cancer is selected from the group consisting of renal cell carcinoma, breast cancer, lung cancer, ovarian cancer, prostate cancer, colon cancer, cervical cancer, brain cancer, liver cancer, pancreatic cancer, kidney and stomach cancer.

The present disclosure includes a type of cellular therapy where γδ T cells are genetically modified to express a TFP. The infused cell is able to kill tumor cells in the recipient. Unlike antibody therapies, modified T cells are able to replicate in vivo resulting in long-term persistence that can lead to sustained tumor control. In various aspects, the T cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The present disclosure also includes a type of cellular therapy where T cells are modified, e.g., by in vitro transcribed RNA, to transiently express a TFP and the modified T cell is infused to a recipient in need thereof. The infused cell is able to kill tumor cells in the recipient. Thus, in various aspects, the T cells administered to the patient, is present for less than one month, e.g., three weeks, two weeks, or one week, after administration of the T cell to the patient.

Without wishing to be bound by any particular theory, the anti-tumor immunity response elicited by the modified T cells may be an active or a passive immune response, or alternatively may be due to a direct vs indirect immune response.

In one aspect, the human modified T cells of the disclosure may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a TFP or iii) cryopreservation of the cells.

Ex vivo procedures are well known in the art and are discussed more fully herein. Briefly, cells are isolated from a mammal (e.g., a human) and genetically modified (i.e., transduced or transfected in vitro) with a vector or circRNA disclosed herein. The modified T cell can be administered to a mammalian recipient to provide a therapeutic benefit. The mammalian recipient may be a human. In preferred embodiments, the modified cell can be allogeneic with respect to the recipient.

The procedure for ex vivo expansion of hematopoietic stem and progenitor cells are described herein and are described in U.S. Pat. No. 5,199,942 and International Patent Publication No. WO/2019/180279, incorporated herein by reference.

In addition to using a cell-based vaccine or circRNA in terms of ex vivo immunization, the present disclosure also provides compositions and methods for in vivo immunization to elicit an immune response directed against an antigen in a patient.

Generally, the cells activated and expanded as described herein may be utilized in the treatment and prevention of diseases that arise in individuals who are immunocompromised.

The modified T cells of the present disclosure may be administered either alone, or as a pharmaceutical composition in combination with diluents and/or with other components such as IL-2 or other cytokines or cell populations.

### Combination Therapies

A modified γδ T cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the “at least one additional therapeutic agent” includes an additional modified T cell, e.g., a modified T cell or a modified γδ T cell. Also provided are γδ T cells that express multiple TFPs, which bind to the same or different target antigens, or same or different epitopes on the same target antigen. Also provided are populations of T cells in which a first subset of T cells express a first TFP and a TCR gamma and/or delta contant domain and a second subset of T cells express a second TFP and a TCR gamma and/or delta contant domain.

A modified γδ T cell described herein and the at least one additional therapeutic agent can be administered simultaneously, in the same or in separate compositions, or sequentially. For sequential administration, the modified T cell described herein can be administered first, and the additional agent can be administered second, or the order of administration can be reversed.

In further aspects, a modified γδ T cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as alemtuzumab, anti-CD3 antibodies or other antibody therapies, cytoxin, fludarabine, cyclosporin, tacrolimus, rapamycin, mycophenolic acid, steroids, romidepsin, cytokines, and irradiation. peptide vaccine, such as that described in Izumoto et al. 2008 J Neurosurg 108:963-971.

In one embodiment, the subject can be administered an agent which reduces or ameliorates a side effect associated with the administration of a modified γδ T cell. Side effects associated with the administration of a modified T cell include but are not limited to cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods disclosed herein can comprise administering a modified T cell described herein to a subject and further administering an agent to manage elevated levels of a soluble factor resulting from treatment with a modified T cell. In one embodiment, the soluble factor elevated in the subject is one or more of IFN-γ, TNFα, IL-2 and IL-6. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is entanercept. An example of an IL-6 inhibitor is tocilizumab (toc).

In one embodiment, the subject can be administered an agent which enhances the activity of a modified γδ T cell. For example, in one embodiment, the agent can be an agent which inhibits an inhibitory molecule. Inhibitory molecules, e.g., Programmed Death 1 (PD1), can, in some embodiments, decrease the ability of a modified T cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize a modified T cell performance. In embodiments, an inhibitory nucleic acid, e.g., an inhibitory nucleic acid, e.g., a dsRNA, e.g., an siRNA or shRNA, can be used to inhibit expression of an inhibitory molecule in the TFP-expressing cell. In an embodiment the inhibitor is a shRNA. In an embodiment, the inhibitory molecule is inhibited within a modified γδ T cell. In these embodiments, a dsRNA molecule that inhibits expression of the inhibitory molecule is linked to the nucleic acid that encodes a component, e.g., all of the components, of the TFP. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3.

In some embodiments, the agent which enhances the activity of a modified γδ T cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the TFP. In another embodiment, the fusion protein is expressed by a cell, e.g., a γδ T cell that does not express an anti-TAA TFP.

Sequence Listing

The human CD3-epsilon polypeptide canonical sequence is:
MQSGTHWRVLGLCLLSVGVWGQDGNEEMGGITQTPYKVSISGTTVILTCPQYPGSEILW
QHNDKNIGGDEDDKNIGSDEDHLSLKEFSELEQSGYYVCYPRGSKPEDANFYLYLRARV
CENCMEMDVMSVATIVIVDICITGGLLLLVYYWSKNRKAKAKPVTRGAGAGGRQRGQ
NKERPPPVPNPDYEPIRKGQRDLYSGLNQRRI (SEQ ID NO: 1).
In one embodiment, the human CD3-epsilon fragment used in the TFPs is
DGNEEMGGITQTPYKVSISGTTVILTCPQYPGSEILWQHNDKNIGGDEDDKNIGSDEDHL
SLKEFSELEQSGYYVCYPRGSKPEDANFYLYLRARVCENCMEMDVMSVATIVIVDICITG
GLLLLVYYWSKNRKAKAKPVTRGAGAGGRQRGQNKERPPPVPNPDYEPIRKGQRDLYS
GLNQRRI (SEQ ID NO: 2)
The human CD3-gamma polypeptide canonical sequence is:
MEQGKGLAVLILAIILLQGTLAQSIKGNHLVKVYDYQEDGSVLLTCDAEAKNITWFKDG
KMIGFLTEDKKKWNLGSNAKDPRGMYQCKGSQNKSKPLQVYYRMCQNCIELNAATISG
FLFAEIVSIFVLAVGVYFIAGQDGVRQSRASDKQTLLPNDQLYQPLKDREDDQYSHLQG
NQLRRN (SEQ ID NO: 3).
In one embodiment, the human CD3-gamma fragment used in the TFPs is:
QSIKGNHLVKVYDYQEDGSVLLTCDAEAKNITWFKDGKMIGFLTEDKKKWNLGSNAK
DPRGMYQCKGSQNKSKPLQVYYRMCQNCIELNAATISGFLFAEIVSIFVLAVGVYFIAGQ
DGVRQSRASDKQTLLPNDQLYQPLKDREDDQYSHLQGNQLRRN (SEQ ID NO: 4).
The human CD3-delta polypeptide canonical sequence is:
MEHSTFLSGLVLATLLSQVSPFKIPIEELEDRVFVNCNTSITWVEGTVGTLLSDITRLDLG
KRILDPRGIYRCNGTDIYKDKESTVQVHYRMCQSCVELDPATVAGIIVTDVIATLLLALG
VFCFAGHETGRLSGAADTQALLRNDQVYQPLRDRDDAQYSHLGGNWARNK (SEQ ID
NO: 5).
In one embodiment, the human CD3-delta fragment used in the TFPs is:
FKIPIEELEDRVFVNCNTSITWVEGTVGTLLSDITRLDLGKRILDPRGIYRCNGTDIYKDKE
STVQVHYRMCQSCVELDPATVAGIIVTDVIATLLLALGVFCFAGHETGRLSGAADTQAL
LRNDQVYQPLRDRDDAQYSHLGGNWARNK (SEQ ID NO: 6)
The human CD3-zeta polypeptide canonical sequence is:
MKWKALFTAAILQAQLPITEAQSFGLLDPKLCYLLDGILFIYGVILTALFLRVKFSRSADA
PAYQQGQNQLYNELNLGRREEYDVLDKRRGRDPEMGGKPQRRKNPQEGLYNELQKDK
MAEAYSEIGMKGERRRGKGHDGLYQGLSTATKDTYDALHMQALPPR (SEQ ID NO: 7).
In one embodiment, the human CD3-zeta fragment used in the TFPs is:
RVKFSRSADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHMQALPPR
(SEQ ID NO: 8)
Anti-CD19
Anti-CD19 light chain CDR1
Coding Sequence: AGGGCAAGTCAGGACATTAGTAAA (SEQ ID NO: 9).
Amino acid sequence: RASQDISK (SEQ ID NO: 10).
Anti-CD19 light chain CDR2
Coding Sequence: ATCTACCATACATCAAGATTA (SEQ ID NO: 11).
Amino acid sequence: IYHTSRL (SEQ ID NO: 12).
Anti-CD19 light chain CDR3
Coding Sequence: CAACAGGGTAATACGCTTCCGTACACG (SEQ ID NO: 13).
Amino acid sequence: QQGNTLPYT (SEQ ID NO: 14).
Anti-CD19 heavy chain CDR1
Coding Sequence: GGGGTCTCATTACCCGACTATGGTGTAAGC (SEQ ID NO: 15).
Amino acid sequence: GVSLPDYGVS (SEQ ID NO: 16).
Anti-CD19 heavy chain CDR2
Coding Sequence: GTAATATGGGGTAGTGAAACCACATACTATAATTCAGCTCTC (SEQ
ID NO: 17).
Amino acid sequence: VIWGSETTYYNSAL (SEQ ID NO: 18).
Anti-CD19 heavy chain CDR3
Coding Sequence: CATTATTACTACGGTGGTAGCTATGCTATGGACTAC (SEQ ID NO:
19).
Amino acid sequence: HYYYGGSYAMDY (SEQ ID NO: 20).
Anti-CD19 light chain variable region
Coding Sequence:
GACATCCAGATGACACAGACTACATCCTCCCTGTCTGCCTCTCTGGGAGACAGAGTC
ACCATCAGTTGCAGGGCAAGTCAGGACATTAGTAAATATTTAAATTGGTATCAGCAG
AAACCAGATGGAACTGTTAAACTCCTGATCTACCATACATCAAGATTACACTCAGGA
GTCCCATCAAGGTTCAGTGGCAGTGGGTCTGGAACAGATTATTCTCTCACCATTAGC
AACCTGGAGCAAGAAGATATTGCCACTTACTTTTGCCAACAGGGTAATACGCTTCCG
TACACGTTCGGAGGGGGGACTAAGTTGGAAATAACA (SEQ ID NO: 21).
Amino acid sequence:
DIQMTQTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEIT(SEQ ID NO: 22).
Anti-CD19 heavy chain variable region
Coding Sequence:
GAGGTGAAACTGCAGGAGTCAGGACCTGGCCTGGTGGCGCCCTCACAGAGCCTGTC
CGTCACATGCACTGTCTCAGGGGTCTCATTACCCGACTATGGTGTAAGCTGGATTCG
CCAGCCTCCACGAAAGGGTCTGGAGTGGCTGGGAGTAATATGGGGTAGTGAAACCA
CATACTATAATTCAGCTCTCAAATCCAGACTGACCATCATCAAGGACAACTCCAAGA
GCCAAGTTTTCTTAAAAATGAACAGTCTGCAAACTGATGACACAGCCATTTACTACT
GTGCCAAACATTATTACTACGGTGGTAGCTATGCTATGGACTACTGGGGTCAAGGAA
CCTCAGTCACCGTCTCCTCA (SEQ ID NO: 23).
Amino acid sequence:
EVKLQESGPGLVAPSQSLSVTCTVSGVSLPDYGVSWIRQPPRKGLEWLGVIWGSETTYY
NSALKSRLTIIKDNSKSQVFLKMNSLQTDDTAIYYCAKHYYYGGSYAMDYWGQGTSVT
VSS (SEQ ID NO: 24).
Anti-MSLN Single Domain Binders
Single domain anti-MSLN binder 1 (SD1)
EVQLVESGGGLVQPGGSLRLSCAASGGDWSANFMYWYRQAPGKQRELVARISGRGVV
DYVESVKGRFTISRDNSKNTLYLQMNSLRAEDTAVYYCAVASYWGQGTLVTVSS (SEQ
ID NO: 25).
SD1 CDR1
GGDWSANFMY (SEQ ID NO: 35)
SD1 CDR2
RISGRGVVDYVESVKGRFT (SEQ ID NO: 36)
SD1 CDR3
ASY (SEQ ID NO: 37)
Single domain anti-MSLN binder 4 (SD4)
EVQLVESGGGLVQPGGSLRLSCAASGSTSSINTMYWYRQAPGKERELVAFISSGGSTNV
RDSVKGRFTISRDNSKNTLYLQMNSLRAEDTAVYYCNTYIPYGGTLHDFWGQGTLVTV
SS (SEQ ID NO: 26).
SD4 CDR1
GSTSSINTMY (SEQ ID NO: 38)
SD4 CDR2
FISSGGSTNVRDSVKGRFT (SEQ ID NO: 39)
SD4 CDR3
YIPYGGTLHDF (SEQ ID NO: 40)
Single domain anti-MSLN binder 6 (SD6)
QVQLVESGGGVVQAGGSLRLSCAASGSTFSIRAMRWYRQAPGTERDLVAVIYGSSTYY
ADAVKGRFTISRDNSKNTLYLQMNSLRAEDTAVYYCNADTIGTARDYWGQGTLVTVSS
(SEQ ID NO: 41)
SD6 CDR1
GSTFSIRAMR (SEQ ID NO: 42)
SD6 CDR2
VIYGSSTYYADAVKGRFT (SEQ ID NO: 43)
SD6 CDR3
DTIGTARDY (SEQ ID NO: 44)
Lentival expression vector with MSLN TFP and IL-15 under T2AW bi-directional promoter
(SEQ ID NO: 29)

| | |
|---|---|
| 1 | TGATCATAAT CAAGCCATAT CACATCTGTA GAGGTTTACT TGCTTTAAAA AACCTCCACA |
| 61 | CCTCCCCCTG AACCTGAAAC ATAAAATGAA TGCAATTGTT GTTGTTAACT TGTTTATTGC |
| 121 | AGCTTATAAT GGTTACAAAT AAAGCAATAG CATCACAAAT TTCACAAATA AAGCATTTTT |
| 181 | TTCACTGCAT TCTAGTTGTG GTTTGTCCAA ACTCATCAAT GTATCTTATC ATGTCTGGAT |
| 241 | CTGCGTCGAC ACGAAGAGAC GACTGACTGA CTGACTGGAA AGAGGAAGGG CTGGAAGAGG |
| 301 | AAGGAGCTTG ATCCAGATCC CGATCTCGAT CCAGATCCGG ATCGCAGCTT GGCGTAATCA |
| 361 | TGGTCATAGC TGTTTCCTGT GTGAAATTGT TATCCGCTCA CAATTCCACA CAACATACGA |
| 421 | GCCGGAAGCA TAAAGTGTAA AGCCTGGGGT GCCTAATGAG TGAGCTAACT CACATTAATT |
| 481 | GCGTTGCGCT CACTGCCCGC TTTCCAGTCG GGAAACCTGT CGTGCCAGCT GCATTAATGA |
| 541 | ATCGGCCAAC GCGCGGGGAG AGGCGGTTTG CGTATTGGGC GCTCTTCCGC TTCCTCGCTC |
| 601 | ACTGACTCGC TGCGCTCGGT CGTTCGGCTG CGGCGAGCGG TATCAGCTCA CTCAAAGGCG |
| 661 | GTAATACGGT TATCCACAGA ATCAGGGGAT AACGCAGGAA AGAACATGTG AGCAAAAGGC |
| 721 | CAGCAAAAGG CCAGGAACCG TAAAAAGGCC GCGTTGCTGG CGTTTTTCCA TAGGCTCCGC |
| 781 | CCCCCTGACG AGCATCACAA AAATCGACGC TCAAGTCAGA GGTGGCGAAA CCCGACAGGA |
| 841 | CTATAAAGAT ACCAGGCGTT TCCCCCTGGA AGCTCCCTCG TGCGCTCTCC TGTTCCGACC |
| 901 | CTGCCGCTTA CCGGATACCT GTCCGCCTTT CTCCCTTCGG GAAGCGTGGC GCTTTCTCAT |
| 961 | AGCTCACGCT GTAGGTATCT CAGTTCGGTG TAGGTCGTTC GCTCCAAGCT GGGCTGTGTG |
| 1021 | CACGAACCCC CCGTTCAGCC CGACCGCTGC GCCTTATCCG GTAACTATCG TCTTGAGTCC |
| 1081 | AACCCGGTAA GACACGACTT ATCGCCACTG GCAGCAGCCA CTGGTAACAG GATTAGCAGA |
| 1141 | GCGAGGTATG TAGGCGGTGC TACAGAGTTC TTGAAGTGGT GGCCTAACTA CGGCTACACT |
| 1201 | AGAAGAACAG TATTTGGTAT CTGCGCTCTG CTGAAGCCAG TTACCTTCGG AAAAAGAGTT |
| 1261 | GGTAGCTCTT GATCCGGCAA ACAAACCACC GCTGGTAGCG GTGGTTTTTT TGTTTGCAAG |
| 1321 | CAGCAGATTA CGCGCAGAAA AAAAGGATCT CAAGAAGATC CTTTGATCTT TTCTACGGGG |
| 1381 | TCTGACGCTC AGTGGAACGA AAACTCACGT TAAGGGATTT TGGTCATGAG ATTATCAAAA |
| 1441 | AGGATCTTCA CCTAGATCCT TTTAAATTAA AAATGAAGTT TTAAATCAAT CTAAAGTATA |
| 1501 | TATGAGTAAA CTTGGTCTGA CAGTTACCAA TGCTTAATCA GTGAGGCACC TATCTCAGCG |
| 1561 | ATCTGTCTAT TTCGTTCATC CATAGTTGCC TGACTCCCCG TCGTTGCTAG GTTACTGTCA |
| 1621 | TGAGCGGATA CATATTTGAA TGTATTTAGA AAAATAAACA AAAGAGTTTG TAGAAACGCA |
| 1681 | AAAAGGCCAT CCGTCAGGAT GGCCTTCTGC TTAATTTGAT CGGTGGCAGT TTATGGCGGG |
| 1741 | CGTCCTGCCC GCCACCCTCC GGGCCGTTGC TTCGCAACGT TCAAATCCGC TCCCGGCGGA |
| 1801 | TTTGTCCTAC TCAGGAGAGC GTTCACCGAC AAACAACAGA TAAAACGAAA GGCCCAGTCT |
| 1861 | TTCGACTGAG CCTTTCGTTT TATTTGATGC CTGGCAGTTC CCTACTCTCG CATGGGTTGC |
| 1921 | GGCCGCCCGG GCCGTCGACC AATTCTCATG TTTGACAGCT TATCATCGAA TTTCTGCCAT |
| 1981 | TCATCCGCTT ATTATCACTT ATTCAGGCGT AGCAACCAGG CGTTTAAGGG CACCAATAAC |
| 2041 | TGCCTTAAAA AAATTACGCC CCGCCCTGCC ACTCATCGCA GTACTGTTGT AATTCATTAA |
| 2101 | GCATTCTGCC GACATGGAAG CCATCACAAA CGGCATGATG AACCTGAATC GCCAGCGGCA |
| 2161 | TCAGCACCTT GTCGCCTTGC GTATAATATT TGCCCATGGT GAAAACGGGG GCGAAGAAGT |
| 2221 | TGTCCATATT GGCCACGTTT AAATCAAAAC TGGTGAAACT CACCCAGGGA TTGGCTGAGA |
| 2281 | CGAAAAACAT ATTCTCAATA AACCCTTTAG GGAAATAGGC CAGGTTTTCA CCGTAACACG |
| 2341 | CCACATCTTG CGAATATATG TGTAGAAACT GCCGGAAATC GTCGTGGTAT TCACTCCAGA |
| 2401 | GCGATGAAAA CGTTTCAGTT TGCTCATGGA AAACGGTGTA ACAAGGGTGA ACACTATCCC |
| 2461 | ATATCACCAG CTCACCGTCT TTCATTGCCA TACGAAATTC CGGATGAGCA TTCATCAGGC |
| 2521 | GGGCAAGAAT GTGAATAAAG GCCGGATAAA ACTTGTGCTT ATTTTTCTTT ACGGTCTTTA |
| 2581 | AAAAGGCCGT AATATCCAGC TGAACGGTCT GGTTATAGGT ACATTGAGCA ACTGACTGAA |
| 2641 | ATGCCTCAAA ATGTTCTTTA CGATGCCATT GGGATATATC AACGGTGGTA TATCCAGTGA |
| 2701 | TTTTTTTCTC CATTTTAGCT TCCTTAGCTC CTGAAAATCT CGATAACTCA AAAAATACGC |
| 2761 | CCGGTAGTGA TCTTATTTCA TTATGGTGAA AGTTGGAACC TCTTACGTGC CGATCAACGT |
| 2821 | CTCATTTTCG CCAAAAGTGA CATTAACCTA TAAAAATAGG CGTATCACGA GGCCAGCTTG |
| 2881 | GGAAACCATA AGACCGAGAT AGAGTTGAGT GTTGTTCCAG TTTGGAACAA GAGTCCACTA |
| 2941 | TTAAAGAACG TGGACTCCAA CGTCAAAGGG CGAAAAACCG TCTATCAGGG CGATGGCCCA |
| 3001 | CTACGTGAAC CATCACCCAA ATCAAGTTTT TTGGGGTCGA GGTGCCGTAA AGCACTAAAT |
| 3061 | CGGAACCCTA AAGGGAGCCC CCGATTTAGA GCTTGACGGG GAAAGCCGGC GAACGTGGCG |
| 3121 | AGAAAGGAAG GGAAGAAAGC GAAAGGAGCG GGCGCTAAGG CGCTGGCAAG TGTAGCGGTC |
| 3181 | ACGCTGCGCG TAACCACCAC ACCCGCCGCG CTTAATGCGC CGCTACAGGG CGCGTACTAT |
| 3241 | GGTTGCTTTG ACGTATGCGG TGTGAAATAC CGCACAGATG CGTAAGGAGA AAATACCGCA |
| 3301 | TCAGGCGCCA TTCGCCATTC AGGCTGCGCA ACTGTTGGGA AGGGCGATCG GTGCGGGCCT |
| 3361 | CTTCGCTATT ACGCCAGCTG GCGAAAGGGG GATGTGCTGC AAGGCGATTA AGTTGGGTAA |
| 3421 | CGCCAGGGTT TTCCCAGTCA CGACGTTGTA AAACGACGGC CAGTGAATTG ATCGAGATCG |
| 3481 | TGATCCGGAT CAAGATCCAG ATCGAATTGG AGGCTACAGT CAGTGGAGAG GACTTTCACT |
| 3541 | GACTGACTGA CTGCGTCTCA ACCTCCTAGG GGACATTGAT TATTGACTAG TTATTAATAG |
| 3601 | TAATCAATTA CGGGGTCATT AGTTCATAGC CCATATATGG AGTTCCGCGT TACATAACTT |
| 3661 | ACGGTAAATG GCCCGCCTGG CTGACCGCCC AACGACCCCC GCCCATTGAC GTCAATAATG |
| 3721 | ACGTATGTTC CCATAGTAAC GCCAATAGGG ACTTTCCATT GACGTCAATG GGTGGAGTAT |
| 3781 | TTACGGTAAA CTGCCCACTT GGCAGTACAT CAAGTGTATC ATATGCCAAG TACGCCCCCT |
| 3841 | ATTGACGTCA ATGACGGTAA ATGGCCCGCC TGGCATTATG CCCAGTACAT GACCTTATGG |
| 3901 | GACTTTCCTA CTTGGCAGTA CATCTACGTA TTAGTCATCG CTATTACCAT GGTGATGCGG |
| 3961 | TTTTGGCAGT ACATCAATGG GCGTGGATAG CGGTTTGACT CACGGGGATT TCCAAGTCTC |
| 4021 | CACCCCATTG ACGTCAATGG GAGTTTGTTT TGGCACCAAA ATCAACGGGA CTTTCCAAAA |
| 4081 | TGTCGTAACA ACTCCGCCCC ATTGACGCAA ATGGGCGGTA GGCGTGTACG GTGGGAGGTC |
| 4141 | TATATAAGCA GAGCTCGTTT AGTGAACCGG GTCTCTCTGG TTAGACCAGA TCTGAGCCTG |
| 4201 | GGAGCTCTCT GGCTAACTAG GGAACCCACT GCTTAAGCCT CAATAAAGCT TGCCTTGAGT |
| 4261 | GCTCAAAGTA GTGTGTGCCC GTCTGTTGTG TGACTCTGGT AACTAGAGAT CCCTCAGACC |
| 4321 | CTTTTAGTCA GTGTGGAAAA TCTCTAGCAG TGGCGCCCGA ACAGGGACTT GAAAGCGAAA |
| 4381 | GTAAAGCCAG AGGAGATCTC TCGACGCAGG ACTCGGCTTG CTGAAGCGCG CACGGCAAGA |
| 4441 | GGCGAGGGGC GGCGACTGGT GAGTACGCCA AAAATTTTGA CTAGCGGAGG CTAGAAGGAG |
| 4501 | AGAGTAGGGT GCGAGAGCGT CGGTATTAAG CGGGGGAGAA TTAGATAAAT GGGAAAAAAT |
| 4561 | TCGGTTAAGG CCAGGGGGAA AGAAACAATA TAAACTAAAA CATATAGTTA GGGCAAGCAG |
| 4621 | GGAGCTAGAA CGATTCGCAG TTAATCCTGG CCTTTTAGAG ACATCAGAAG GCTGTAGACA |
| 4681 | AATACTGGGA CAGCTACAAC CATCCCTTCA GACAGGATCA GAAGAACTTA GATCATTATA |
| 4741 | TAATACAATA GCAGTCCTCT ATTGTGTGCA TCAAAGGATA GATGTAAAAG ACACCAAGGA |
| 4801 | AGCCTTAGAT AAGATAGAGG AAGAGCAAAA CAAAAGTAAG AAAAAGGCAC AGCAAGCGAT |
| 4861 | CTTCAGACCT GGAGGAGGCA GGAGGCGATA TGAGGGACAA TTGGAGAAGT GAATTATATA |
| 4921 | AATATAAAGT AGTAAAAATT GAACCATTAG GAGTAGCACC CACCAAGGCA AAGAGAAGAG |
| 4981 | TGGTGCAGAG AGAAAAAAGA GCAGTGGGAA TAGGAGCTTT GTTCCTTGGG TTCTTGGGAG |
| 5041 | CAGCAGGAAG CACTATGGGC GCAGCGTCAA TGACGCTGAC GGTACAGGCC AGACAATTAT |
| 5101 | TGTCTGATAT AGTGCAGCAG CAGAACAATT TGCTGAGGGC TATTGAGGCG CAACAGCATC |
| 5161 | TGTTGCAACT CACAGTCTGG GGCATCAAAC AGCTCCAGGC AAGAATCCTG GCTGTGGAAA |
| 5221 | GATACCTAAA GGATCAACAG CTCCTGGGGA TTTGGGGTTG CTCTGGAAAA CTCATTTGCA |
| 5281 | CCACTGCTGT GCCTTGGAAT GCTAGTTGGA GTAATAAATC TCTGGAACAG ATTTGGAATA |
| 5341 | ACATGACCTG GATGGAGTGG GACAGAGAAA TTAACAATTA CACAAGCTTA ATACACTCCT |
| 5401 | TAATTGAAGA ATCGCAAAAC CAGCAAGAAA AGAATGAACA AGAATTATTG GAATTAGATA |
| 5461 | AATGGGCAAG TTTGTGGAAT TGGTTTAACA TAACAAATTG GCTGTGGTAT ATAAAATTAT |
| 5521 | TCATAATGAT AGTAGGAGGC TTGGTAGGTT TAAGAATAGT TTTTGCTGTA CTTTCTATAG |
| 5581 | TGAATAGAGT TAGGCAGGGA TATTCACCAT TATCGTTTCA GACCCACCTC CCAATCCCGA |
| 5641 | GGGGACCACG CGTACAAATG GCAGTATTCA TCCACAATTT TAAAAGAAAA GGGGGGATTG |
| 5701 | GGGGGTACAG TGCAGGGGAA AGAATAGTAG ACATAATAGC AACAGACATA CAAACTAAAG |
| 5761 | AATTACAAAA ACAAATTACA AAAATTCAAA ATTTTCGGGT TTATTACAGG GACAGCAGAA |
| 5821 | ATCCACTTTG GAAAGCTGAG CATCCGGCTC CGGTGCCCGT CAGTGGGCAG AGCGCACATC |
| 5881 | GCCCACAGTC CCCGAGAAGT TGGGGGGAGG GGTCGGCAAT TGAACCGGTG CCTAGAGAAG |
| 5941 | GTGGCGCGGG GTAAACTGGG AAAGTGATGT CGTGTACTGG CTCCGCCTTT TTCCCGAGGG |
| 6001 | TGGGGGAGAA CCGTATATAA GTGCAGTAGT CGCCGTGAAC GTTCTTTTTC GCAACGGGTT |
| 6061 | TGCCGCCAGA ACACAGGTAA GTGCCGTGTG TGGTTCCCGC GGGCCTGGCC TCTTTACGGG |
| 6121 | TTATGGCCCT TGCGTGCCTT GAATTACTTC CACGCCCCTG GCTGCAGTAC GTGATTCTTG |
| 6181 | ATCCCGAGCT TCGGGTTGGA AGTGGGTGGG AGAGTTCGAG GCCTTGCGCT TAAGGAGCCC |
| 6241 | CTTCGCCTCG TGCTTGAGTT GAGGCCTGGC CTGGGCGCTG GGGCCGCCGC GTGCGAATCT |
| 6301 | GGTGGCACCT TCGCGCCTGT CTCGCTGCTT TCGATAAGTC TCTAGCCATT TAAAATTTTT |
| 6361 | GATGACCTGC TGCGACGCTT TTTTTCTGGC AAGATAGTCT TGTAAATGCG GGCCAAGATC |
| 6421 | TGCACACTGG TATTTCGGTT TTTGGGGCCG CGGGCGGCGA CGGGGCCCGT GCGTCCCAGC |
| 6481 | GCACATGTTC GGCGAGGCGG GGCCTGCGAG CGCGGCCACC GAGAATCGGA CGGGGGTAGT |
| 6541 | CTCAAGCTGG CCGGCCTGCT CTGGTGCCTG GCCTCGCGCC GCCGTGTATC GCCCCGCCCT |
| 6601 | GGGCGGCAAG GCTGGCCCGG TCGGCACCAG TTGCGTGAGC GGAAAGATGG CCGCTTCCCG |
| 6661 | GCCCTGCTGC AGGGAGCTCA AAATGGAGGA CGCGGCGCTC GGGAGAGCGG GCGGGTGAGT |
| 6721 | CACCCACACA AAGGAAAAGG GCCTTTCCGT CCTCAGCCGT CGCTTCATGT GACTCCACGG |
| 6781 | AGTACCGGGC GCCGTCCAGG CACCTCGATT AGTTCTCGAG CTTTTGGAGT ACGTCGTCTT |
| 6841 | TAGGTTGGGG GGAGGGGTTT TATGCGATGG AGTTTCCCCA CACTGAGTGG GTGGAGACTG |
| 6901 | AAGTTAGGCC AGCTTGGCAC TTGATGTAAT TCTCCTTGGA ATTTGCCCTT TTTGAGTTTG |
| 6961 | GATCTTGGTT CATTCTCAAG CCTCAGACAG TGGTTCAAAG TTTTTTTCTT CCATTTCAGG |
| 7021 | TGTCGTGAAA ACTACCCCTC AGAGCCGCCA CCATGCTTCT GCTTGTGACG TCCCTCCTGC |
| 7081 | TTTGTGAACT CCCGCATCCT GCCTTCCTGC TCATCCCGGA AGTTCAGCTG GTAGAGTCCG |
| 7141 | GTGGCGGGCT GGTACAGCCA GGGGGATCTC TCCGTCTCTC ATGTGCTGCC AGTGGGGGAG |
| 7201 | ATTGGTCCGC TAACTTTATG TACTGGTATC GTCAAGCACC TGGGAAACAA CGCGAGCTGG |
| 7261 | TTGCACGGAT CTCTGGACGA GGGGTAGTGG ATTACGTGGA AAGTGTGAAA GGACGATTCA |
| 7321 | CAATTTCCAG GGACAATTCT AAAAACACCC TTTATTTGCA AATGAATAGT CTGAGAGCAG |
| 7381 | AAGACACAGC CGTATATTAT TGTGCAGTCG CATCCTATTG GGGGCAGGGG ACTCTTGTCA |
| 7441 | CAGTCTCGAG TGCTGCAGCA GGCGGCGGGG GCTCAGGAGG GGGTGGGTCT GGAGGAGGTG |
| 7501 | GGAGTCTGGA GGATGGTAAT GAAGAAATGG GTGGTATTAC ACAGACACCA TATAAAGTCT |
| 7561 | CCATCTCTGG AACCACAGTA ATATTGACAT GCCCTCAGTA TCCTGGATCT GAAATACTAT |
| 7621 | GGCAACACAA TGATAAAAAC ATAGGCGGTG ATGAGGATGA TAAAAACATA GGCAGTGATG |
| 7681 | AGGATCACCT GTCACTGAAG GAATTTTCAG AATTGGAGCA AAGTGGTTAT TATGTCTGCT |
| 7741 | ACCCCAGAGG AAGCAAACCA GAAGATGCGA ACTTTTATCT CTACCTGAGG GCAAGAGTGT |
| 7801 | GTGAGAACTG CATGGAGATG GATGTGATGT CGGTGGCCAC AATTGTCATA GTGGACATCT |
| 7861 | GCATCACTGG GGGCTTGCTG CTGCTGGTTT ACTACTGGAG CAAGAATAGA AAGGCCAAGG |
| 7921 | CCAAGCCTGT GACACGAGGA GCGGGTGCTG GCGGCAGGCA AAGGGGACAA AACAAGGAGA |
| 7981 | GGCCACCACC TGTTCCCAAC CCAGACTATG AGCCCATCCG GAAAGGCCAG CGGGACCTGT |
| 8041 | ATTCTGGCCT GAATCAGAGA CGCATCGGGT CAGGCGAGGG CAGAGGAAGT CTGCTAACAT |
| 8101 | GCGGTGACGT CGAGGAGAAT CCTGGACCTA TGAGAATCAG CAAGCCTCAC CTGAGAAGCA |
| 8161 | TCTCCATTCA GTGCTACCTG TGCCTCCTTC TGAACAGCCA CTTCCTGACC GAGGCTGGCA |
| 8221 | TTCACGTGTT CATCCTGGGC TGCTTCAGCG CAGGACTGCC CAAGACCGAG GCTAACTGGG |
| 8281 | TTAACGTGAT CAGCGACCTG AAGAAAATCG AGGACCTGAT TCAGAGCATG CACATCGACG |
| 8341 | CTACCCTGTA CACCGAGAGC GATGTGCATC CTTCTTGCAA GGTGACAGCC ATGAAGTGCT |
| 8401 | TCCTGCTGGA ACTCCAGGTG ATCTCTCTGG AGAGCGGAGA TGCCAGCATT CACGACACAG |
| 8461 | TGGAAAACCT GATCATCCTG GCCAATAACT CCCTGTCTAG CAACGGAAAT GTGACCGAAA |
| 8521 | GCGGCTGTAA AGAGTGCGAA GAGCTGGAGG AAAAGAACAT CAAAGAGTTC CTGCAGAGCT |
| 8581 | TCGTGCATAT CGTGCAGATG TTCATCAACA CCTCTTGAGA TATCGAGCAT CTTACCGCCA |
| 8641 | TTTATACCCA TATTTGTTCT GTTTTTCTTG ATTTGGGTAT ACATTTAAAT GTTAATAAAA |
| 8701 | CAAAATGGTG GGGCAATCAT TTACATTTTT AGGGATATGT AATTACTAGT TCAGGTGTAT |
| 8761 | TGCCACAAGA CAAACATGTT AAGAAACTTT CCCGTTATTT ACGCTCTGTT CCTGTTAATC |
| 8821 | AACCTCTGGA TTACAAAATT TGTGAAAGAT TGACTGATAT TCTTAACTAT GTTGCTCCTT |
| 8881 | TTACGCTGTG TGGATATGCT GCTTTATAGC CTCTGTATCT AGCTATTGCT TCCCGTACGG |
| 8941 | CTTTCGTTTT CTCCTCCTTG TATAAATCCT GGTTGCTGTC TCTTTTAGAG GAGTTGTGGC |
| 9001 | CCGTTGTCCG TCAACGTGGC GTGGTGTGCT CTGTGTTTGC TGACGCAACC CCCACTGGCT |
| 9061 | GGGGCATTGC CACCACCTGT CAACTCCTTT CTGGGACTTT CGCTTTCCCC CTCCCGATCG |
| 9121 | CCACGGCAGA ACTCATCGCC GCCTGCCTTG CCCGCTGCTG GACAGGGGCT AGGTTGCTGG |
| 9181 | GCACTGATAA TTCCGTGGTG TTGTCAGTAC TGGTACCTTT AAGACCAATG ACTTACAAGG |
| 9241 | CAGCTGTAGA TCTTAGCCAC TTTTTAAAAG AAAAGGGGGG ACTGGAAGGG CTAATTCACT |
| 9301 | CCCAAAGAAG ACAAGATCTG CTTTTTGCCT GTACTGGGTC TCTCTGGTTA GACCAGATCT |
| 9361 | GAGCCTGGGA GCTCTCTGGC TAACTAGGGA ACCCACTGCT TAAGCCTCAA TAAAGCTTGC |
| 9421 | CTTGAGTGCT TCAA |

Lentival expression vector with CD 19 TFP and IL-15 under T2AW bi-directional promoter
(SEQ ID NO: 30)

| | |
|---|---|
| 1 | TGATCATAAT CAAGCCATAT CACATCTGTA GAGGTTTACT TGCTTTAAAA AACCTCCACA |
| 61 | CCTCCCCCTG AACCTGAAAC ATAAAATGAA TGCAATTGTT GTTGTTAACT TGTTTATTGC |
| 121 | AGCTTATAAT GGTTACAAAT AAAGCAATAG CATCACAAAT TTCACAAATA AAGCATTTTT |
| 181 | TTCACTGCAT TCTAGTTGTG GTTTGTCCAA ACTCATCAAT GTATCTTATC ATGTCTGGAT |
| 241 | CTGCGTCGAC ACGAAGAGAC GACTGACTGA CTGACTGGAA AGAGGAAGGG CTGGAAGAGG |
| 301 | AAGGAGCTTG ATCCAGATCC CGATCTCGAT CCAGATCCGG ATCGCAGCTT GGCGTAATCA |
| 361 | TGGTCATAGC TGTTTCCTGT GTGAAATTGT TATCCGCTCA CAATTCCACA CAACATACGA |
| 421 | GCCGGAAGCA TAAAGTGTAA AGCCTGGGGT GCCTAATGAG TGAGCTAACT CACATTAATT |
| 481 | GCGTTGCGCT CACTGCCCGC TTTCCAGTCG GGAAACCTGT CGTGCCAGCT GCATTAATGA |
| 541 | ATCGGCCAAC GCGCGGGGAG AGGCGGTTTG CGTATTGGGC GCTCTTCCGC TTCCTCGCTC |
| 601 | ACTGACTCGC TGCGCTCGGT CGTTCGGCTG CGGCGAGCGG TATCAGCTCA CTCAAAGGCG |
| 661 | GTAATACGGT TATCCACAGA ATCAGGGGAT AACGCAGGAA AGAACATGTG AGCAAAAGGC |
| 721 | CAGCAAAAGG CCAGGAACCG TAAAAAGGCC GCGTTGCTGG CGTTTTTCCA TAGGCTCCGC |
| 781 | CCCCCTGACG AGCATCACAA AAATCGACGC TCAAGTCAGA GGTGGCGAAA CCCGACAGGA |
| 841 | CTATAAAGAT ACCAGGCGTT TCCCCCTGGA AGCTCCCTCG TGCGCTCTCC TGTTCCGACC |
| 901 | CTGCCGCTTA CCGGATACCT GTCCGCCTTT CTCCCTTCGG GAAGCGTGGC GCTTTCTCAT |
| 961 | AGCTCACGCT GTAGGTATCT CAGTTCGGTG TAGGTCGTTC GCTCCAAGCT GGGCTGTGTG |
| 1021 | CACGAACCCC CCGTTCAGCC CGACCGCTGC GCCTTATCCG GTAACTATCG TCTTGAGTCC |
| 1081 | AACCCGGTAA GACACGACTT ATCGCCACTG GCAGCAGCCA CTGGTAACAG GATTAGCAGA |
| 1141 | GCGAGGTATG TAGGCGGTGC TACAGAGTTC TTGAAGTGGT GGCCTAACTA CGGCTACACT |
| 1201 | AGAAGAACAG TATTTGGTAT CTGCGCTCTG CTGAAGCCAG TTACCTTCGG AAAAAGAGTT |
| 1261 | GGTAGCTCTT GATCCGGCAA ACAAACCACC GCTGGTAGCG GTGGTTTTTT TGTTTGCAAG |
| 1321 | CAGCAGATTA CGCGCAGAAA AAAAGGATCT CAAGAAGATC CTTTGATCTT TTCTACGGGG |
| 1381 | TCTGACGCTC AGTGGAACGA AAACTCACGT TAAGGGATTT TGGTCATGAG ATTATCAAAA |
| 1441 | AGGATCTTCA CCTAGATCCT TTTAAATTAA AAATGAAGTT TTAAATCAAT CTAAAGTATA |
| 1501 | TATGAGTAAA CTTGGTCTGA CAGTTACCAA TGCTTAATCA GTGAGGCACC TATCTCAGCG |
| 1561 | ATCTGTCTAT TTCGTTCATC CATAGTTGCC TGACTCCCCG TCGTGTAGAT AACTACGATA |
| 1621 | CGGGAGGGCT TACCATCTGG CCCCAGTGCT GCAATGATAC CGCAGCTTGG GAAACCATAA |
| 1681 | GAGCTGAAGC CAGTTACCTT CGGAAAAAGA GTTGGTAGCT CTTGATCCGG CAAACAAACC |
| 1741 | ACCGCTGGTA GCGGTGGTTT TTTTGTTTGC AAGCAGCAGA TTACGCGCAG AAAAAAAGGA |
| 1801 | TCTCAAGAAG ATCCTTTGAT CTTTTCTACG GGGTCTGACG CTCAGTGGAA CGAAAACTCA |
| 1861 | CGTTAAGGGA TTTTGGTCAT GAGCTTGCGC CGTCCCGTCA AGTCAGCGTA ATGCTCTGCC |
| 1921 | AGTGTTACAA CCAATTAACC AATTCTGATT AGAAAAACTC ATCGAGCATC AAATGAAACT |
| 1981 | GCAATTTATT CACATCAGGA TTATCAATAC CATATTTTTG AAAAAGCCGT TTCTGTAATG |
| 2041 | AAGGAGAAAA CTCACCGAGG CAGTTCCATA GGATGGCAAG ATCCTGGTAT CGGTCTGCGA |
| 2101 | TTCCGACTCG TCCAACATCA ATACAACCTA TTAATTTCCC CTCGTCAAAA ATAAGGTTAT |
| 2161 | CAAGTGAGAA ATCACCATGA GTGACGACTG AATCCGGTGA GAATGGCAAA AGTTTATGCA |
| 2221 | TTTCTTTCCA GACTTGTTCA ACAGGCCAGC CATTACGCTC GTCATCAAAA TCACTCGCAT |
| 2281 | CAACCAAACC GTTATTCATT CGTGATTGCG CCTGAGCAAG ACGAAATACG CGATCGCTGT |
| 2341 | TAAAAGGACA ATTACAAACA GGAATCGAAT GCAACCGGCG CAGGAACACT GCCAGCGCAT |
| 2401 | CAACAATATT TTCACCTGAA TCAGGATATT CTTCTAATAC CTGGAATGCT GTTTTTCCGG |
| 2461 | GGATCGCAGT GGTGAGTAAC CATGCATCAT CAGGAGTACG GATAAAATGC TTGATGGTCG |
| 2521 | GAAGAGGCAT AAATTCCGTC AGCCAGTTTA GTCTGACCAT CTCATCTGTA ACATCATTGG |
| 2581 | CAACGCTACC TTTGCCATGT TTCAGAAACA ACTCTGGCGC ATCGGGCTTC CCATACAAGC |
| 2641 | GATAGATTGT CGCACCTGAT TGCCCGACAT TATCGCGAGC CCATTTATAC CCATATAAAT |
| 2701 | CAGCATCCAT GTTGGAATTT AATCGCGGCC TCGACGTTTC CCGTTGAATA TGGCTCATAA |
| 2761 | CACCCCTTGT ATTACTGTTT ATGTAAGCAG ACAGTTTTAT TGTTCATGAT GATATATTTT |
| 2821 | TATCTTGTGC AATGTAACAT CAGAGATTTT GAGACACAAC GTGGCTTTCC CCCCCCCCCC |
| 2881 | CATGACATTA ACCTATAAAA ATAGGCGTAT CACGAGGCCA GCTTGGGAAA CCATAAGACC |
| 2941 | GAGATAGAGT TGAGTGTTGT TCCAGTTTGG AACAAGAGTC CACTATTAAA GAACGTGGAC |
| 3001 | TCCAACGTCA AAGGGCGAAA AACCGTCTAT CAGGGCGATG GCCCACTACG TGAACCATCA |
| 3061 | CCCAAATCAA GTTTTTTGGG GTCGAGGTGC CGTAAAGCAC TAAATCGGAA CCCTAAAGGG |
| 3121 | AGCCCCCGAT TTAGAGCTTG ACGGGGAAAG CCGGCGAACG TGGCGAGAAA GGAAGGGAAG |
| 3181 | AAAGCGAAAG GAGCGGGCGC TAAGGCGCTG GCAAGTGTAG CGGTCACGCT GCGCGTAACC |
| 3241 | ACCACACCCG CCGCGCTTAA TGCGCCGCTA CAGGGCGCGT ACTATGGTTG CTTTGACGTA |
| 3301 | TGCGGTGTGA AATACCGCAC AGATGCGTAA GGAGAAAATA CCGCATCAGG CGCCATTCGC |
| 3361 | CATTCAGGCT GCGCAACTGT TGGGAAGGGC GATCGGTGCG GGCCTCTTCG CTATTACGCC |
| 3421 | AGCTGGCGAA AGGGGGATGT GCTGCAAGGC GATTAAGTTG GGTAACGCCA GGGTTTTCCC |
| 3481 | AGTCACGACG TTGTAAAACG ACGGCCAGTG AATTGATCGA GATCGTGATC CGGATCAAGA |
| 3541 | TCCAGATCGA ATTGGAGGCT ACAGTCAGTG GAGAGGACTT TCACTGACTG ACTGACTGCG |
| 3601 | TCTCAACCTC CTAGGGGACA TTGATTATTG ACTAGTTATT AATAGTAATC AATTACGGGG |
| 3661 | TCATTAGTTC ATAGCCCATA TATGGAGTTC CGCGTTACAT AACTTACGGT AAATGGCCCG |
| 3721 | CCTGGCTGAC CGCCCAACGA CCCCCGCCCA TTGACGTCAA TAATGACGTA TGTTCCCATA |
| 3781 | GTAACGCCAA TAGGGACTTT CCATTGACGT CAATGGGTGG AGTATTTACG GTAAACTGCC |
| 3841 | CACTTGGCAG TACATCAAGT GTATCATATG CCAAGTACGC CCCCTATTGA CGTCAATGAC |
| 3901 | GGTAAATGGC CCGCCTGGCA TTATGCCCAG TACATGACCT TATGGGACTT TCCTACTTGG |
| 3961 | CAGTACATCT ACGTATTAGT CATCGCTATT ACCATGGTGA TGCGGTTTTG GCAGTACATC |
| 4021 | AATGGGCGTG GATAGCGGTT TGACTCACGG GGATTTCCAA GTCTCCACCC CATTGACGTC |
| 4081 | AATGGGAGTT TGTTTTGGCA CCAAAATCAA CGGGACTTTC CAAAATGTCG TAACAACTCC |
| 4141 | GCCCCATTGA CGCAAATGGG CGGTAGGCGT GTACGGTGGG AGGTCTATAT AAGCAGAGCT |
| 4201 | CGTTTAGTGA ACCGGGTCTC TCTGGTTAGA CCAGATCTGA GCCTGGGAGC TCTCTGGCTA |
| 4261 | ACTAGGGAAC CCACTGCTTA AGCCTCAATA AAGCTTGCCT TGAGTGCTCA AAGTAGTGTG |
| 4321 | TGCCCGTCTG TTGTGTGACT CTGGTAACTA GAGATCCCTC AGACCCTTTT AGTCAGTGTG |
| 4381 | GAAAATCTCT AGCAGTGGCG CCCGAACAGG GACTTGAAAG CGAAAGTAAA GCCAGAGGAG |
| 4441 | ATCTCTCGAC GCAGGACTCG GCTTGCTGAA GCGCGCACGG CAAGAGGCGA GGGGCGGCGA |
| 4501 | CTGGTGAGTA CGCCAAAAAT TTTGACTAGC GGAGGCTAGA AGGAGAGAGT AGGGTGCGAG |
| 4561 | AGCGTCGGTA TTAAGCGGGG GAGAATTAGA TAAATGGGAA AAAATTCGGT TAAGGCCAGG |
| 4621 | GGGAAAGAAA CAATATAAAC TAAAACATAT AGTTAGGGCA AGCAGGGAGC TAGAACGATT |
| 4681 | CGCAGTTAAT CCTGGCCTTT TAGAGACATC AGAAGGCTGT AGACAAATAC TGGGACAGCT |
| 4741 | ACAACCATCC CTTCAGACAG GATCAGAAGA ACTTAGATCA TTATATAATA CAATAGCAGT |
| 4801 | CCTCTATTGT GTGCATCAAA GGATAGATGT AAAAGACACC AAGGAAGCCT TAGATAAGAT |
| 4861 | AGAGGAAGAG CAAAACAAAA GTAAGAAAAA GGCACAGCAA GCGATCTTCA GACCTGGAGG |
| 4921 | AGGCAGGAGG CGATATGAGG GACAATTGGA GAAGTGAATT ATATAAATAT AAAGTAGTAA |
| 4981 | AAATTGAACC ATTAGGAGTA GCACCCACCA AGGCAAAGAG AAGAGTGGTG CAGAGAGAAA |
| 5041 | AAAGAGCAGT GGGAATAGGA GCTTTGTTCC TTGGGTTCTT GGGAGCAGCA GGAAGCACTA |
| 5101 | TGGGCGCAGC GTCAATGACG CTGACGGTAC AGGCCAGACA ATTATTGTCT GATATAGTGC |
| 5161 | AGCAGCAGAA CAATTTGCTG AGGGCTATTG AGGCGCAACA GCATCTGTTG CAACTCACAG |
| 5221 | TCTGGGGCAT CAAACAGCTC CAGGCAAGAA TCCTGGCTGT GGAAAGATAC CTAAAGGATC |
| 5281 | AACAGCTCCT GGGGATTTGG GGTTGCTCTG GAAAACTCAT TTGCACCACT GCTGTGCCTT |
| 5341 | GGAATGCTAG TTGGAGTAAT AAATCTCTGG AACAGATTTG GAATAACATG ACCTGGATGG |
| 5401 | AGTGGGACAG AGAAATTAAC AATTACACAA GCTTAATACA CTCCTTAATT GAAGAATCGC |
| 5461 | AAAACCAGCA AGAAAAGAAT GAACAAGAAT TATTGGAATT AGATAAATGG GCAAGTTTGT |
| 5521 | GGAATTGGTT TAACATAACA AATTGGCTGT GGTATATAAA ATTATTCATA ATGATAGTAG |
| 5581 | GAGGCTTGGT AGGTTTAAGA ATAGTTTTTG CTGTACTTTC TATAGTGAAT AGAGTTAGGC |
| 5641 | AGGGATATTC ACCATTATCG TTTCAGACCC ACCTCCCAAT CCCGAGGGGA CCACGCGTAC |
| 5701 | AAATGGCAGT ATTCATCCAC AATTTTAAAA GAAAAGGGGG GATTGGGGGG TACAGTGCAG |
| 5761 | GGGAAAGAAT AGTAGACATA ATAGCAACAG ACATACAAAC TAAAGAATTA CAAAAACAAA |
| 5821 | TTACAAAAAT TCAAAATTTT CGGGTTTATT ACAGGGACAG CAGAAATCCA CTTTGGAAAG |
| 5881 | CTGAGCATCC GGCTCCGGTG CCCGTCAGTG GGCAGAGCGC ACATCGCCCA CAGTCCCCGA |
| 5941 | GAAGTTGGGG GGAGGGGTCG GCAATTGAAC CGGTGCCTAG AGAAGGTGGC GCGGGGTAAA |
| 6001 | CTGGGAAAGT GATGTCGTGT ACTGGCTCCG CCTTTTTCCC GAGGGTGGGG GAGAACCGTA |
| 6061 | TATAAGTGCA GTAGTCGCCG TGAACGTTCT TTTTCGCAAC GGGTTTGCCG CCAGAACACA |
| 6121 | GGTAAGTGCC GTGTGTGGTT CCCGCGGGCC TGGCCTCTTT ACGGGTTATG GCCCTTGCGT |
| 6181 | GCCTTGAATT ACTTCCACGC CCCTGGCTGC AGTACGTGAT TCTTGATCCC GAGCTTCGGG |
| 6241 | TTGGAAGTGG GTGGGAGAGT TCGAGGCCTT GCGCTTAAGG AGCCCCTTCG CCTCGTGCTT |
| 6301 | GAGTTGAGGC CTGGCCTGGG CGCTGGGGCC GCCGCGTGCG AATCTGGTGG CACCTTCGCG |
| 6361 | CCTGTCTCGC TGCTTTCGAT AAGTCTCTAG CCATTTAAAA TTTTTGATGA CCTGCTGCGA |
| 6421 | CGCTTTTTTT CTGGCAAGAT AGTCTTGTAA ATGCGGGCCA AGATCTGCAC ACTGGTATTT |
| 6481 | CGGTTTTTGG GGCCGCGGGC GGCGACGGGG CCCGTGCGTC CCAGCGCACA TGTTCGGCGA |
| 6541 | GGCGGGGCCT GCGAGCGCGG CCACCGAGAA TCGGACGGGG GTAGTCTCAA GCTGGCCGGC |
| 6601 | CTGCTCTGGT GCCTGGCCTC GCGCCGCCGT GTATCGCCCC GCCCTGGGCG GCAAGGCTGG |
| 6661 | CCCGGTCGGC ACCAGTTGCG TGAGCGGAAA GATGGCCGCT TCCCGGCCCT GCTGCAGGGA |
| 6721 | GCTCAAAATG GAGGACGCGG CGCTCGGGAG AGCGGGCGGG TGAGTCACCC ACACAAAGGA |
| 6781 | AAAGGGCCTT TCCGTCCTCA GCCGTCGCTT CATGTGACTC CACGGAGTAC CGGGCGCCGT |
| 6841 | CCAGGCACCT CGATTAGTTC TCGAGCTTTT GGAGTACGTC GTCTTTAGGT TGGGGGGAGG |
| 6901 | GGTTTTATGC GATGGAGTTT CCCCACACTG AGTGGGTGGA GACTGAAGTT AGGCCAGCTT |
| 6961 | GGCACTTGAT GTAATTCTCC TTGGAATTTG CCCTTTTTGA GTTTGGATCT TGGTTCATTC |
| 7021 | TCAAGCCTCA GACAGTGGTT CAAAGTTTTT TTCTTCCATT TCAGGTGTCG TGAAAACTAC |
| 7081 | CCCTCAGAGC CGCCACCATG CTTCTCCTGG TGACAAGCCT TCTGCTCTGT GAGTTACCAC |
| 7141 | ACCCAGCATT CCTCCTGATC CCAGACATCC AGATGACACA GACTACATCC TCCCTGTCTG |
| 7201 | CCTCTCTGGG AGACAGAGTC ACCATCAGTT GCAGGGCAAG TCAGGACATT AGTAAATATT |
| 7261 | TAAATTGGTA TCAGCAGAAA CCAGATGGAA CTGTTAAACT CCTGATCTAC CATACATCAA |
| 7321 | GATTACACTC AGGAGTCCCA TCAAGGTTCA GTGGCAGTGG GTCTGGAACA GATTATTCTC |
| 7381 | TCACCATTAG CAACCTGGAG CAAGAAGATA TTGCCACTTA CTTTTGCCAA CAGGGTAATA |
| 7441 | CGCTTCCGTA CACGTTCGGA GGGGGGACTA AGTTGGAAAT AACAGGCTCC ACCTCTGGAT |
| 7501 | CCGGCAAGCC CGGATCTGGC GAGGGATCCA CCAAGGGCGA GGTGAAACTG CAGGAGTCAG |
| 7561 | GACCTGGCCT GGTGGCGCCC TCACAGAGCC TGTCCGTCAC ATGCACTGTC TCAGGGGTCT |
| 7621 | CATTACCCGA CTATGGTGTA AGCTGGATTC GCCAGCCTCC ACGAAAGGGT CTGGAGTGGC |
| 7681 | TGGGAGTAAT ATGGGGTAGT GAAACCACAT ACTATAATTC AGCTCTCAAA TCCAGACTGA |
| 7741 | CCATCATCAA GGACAACTCC AAGAGCCAAG TTTTCTTAAA AATGAACAGT CTGCAAACTG |
| 7801 | ATGACACAGC CATTTACTAC TGTGCCAAAC ATTATTACTA CGGTGGTAGC TATGCTATGG |
| 7861 | ACTACTGGGG TCAAGGAACC TCAGTCACCG TCTCCTCAGC TGCCGCAGGA GGCGGTGGCA |
| 7921 | GTGGTGGGGG AGGATCTGGC GGAGGAGGTA GCTTGGAAGA TGGTAATGAA GAAATGGGTG |
| 7981 | GTATTACACA GACACCATAT AAAGTCTCCA TCTCTGGAAC CACAGTAATA TTGACATGCC |
| 8041 | CTCAGTATCC TGGATCTGAA ATACTATGGC AACACAACGA TAAGAATATA GGAGGTGATG |
| 8101 | AGGATGATAA AAACATAGGC AGTGATGAGG ATCACCTGTC ACTGAAGGAA TTTTCAGAAT |
| 8161 | TGGAGCAAAG TGGTTATTAT GTCTGCTACC CCAGAGGAAG CAAACCAGAA GATGCGAACT |
| 8221 | TTTATCTCTA CCTGAGGGCA AGAGTGTGTG AGAACTGCAT GGAGATGGAT GTGATGTCGG |
| 8281 | TGGCCACAAT TGTCATAGTG GACATCTGCA TCACTGGGGG CTTGCTGCTG CTGGTTTACT |
| 8341 | ACTGGAGCAA GAATAGAAAG GCCAAGGCCA AGCCTGTGAC ACGAGGAGCG GGTGCTGGCG |
| 8401 | GCAGGCAAAG GGGACAAAAC AAGGAGAGGC CACCACCTGT TCCCAACCCA GACTATGAGC |
| 8461 | CCATCCGGAA AGGCCAGCGG GACCTGTATT CTGGCCTGAA TCAGAGACGC ATCGGGTCAG |
| 8521 | GCGAGGGCAG AGGAAGTCTG CTAACATGCG GTGACGTCGA GGAGAATCCT GGACCTATGA |
| 8581 | GAATCAGCAA GCCTCACCTG AGAAGCATCT CCATTCAGTG CTACCTGTGC CTCCTTCTGA |
| 8641 | ACAGCCACTT CCTGACCGAG GCTGGCATTC ACGTGTTCAT CCTGGGCTGC TTCAGCGCAG |
| 8701 | GACTGCCCAA GACCGAGGCT AACTGGGTTA ACGTGATCAG CGACCTGAAG AAAATCGAGG |
| 8761 | ACCTGATTCA GAGCATGCAC ATCGACGCTA CCCTGTACAC CGAGAGCGAT GTGCATCCTT |
| 8821 | CTTGCAAGGT GACAGCCATG AAGTGCTTCC TGCTGGAACT CCAGGTGATC TCTCTGGAGA |
| 8881 | GCGGAGATGC CAGCATTCAC GACACAGTGG AAAACCTGAT CATCCTGGCC AATAACTCCC |
| 8941 | TGTCTAGCAA CGGAAATGTG ACCGAAAGCG GCTGTAAAGA GTGCGAAGAG CTGGAGGAAA |
| 9001 | AGAACATCAA AGAGTTCCTG CAGAGCTTCG TGCATATCGT GCAGATGTTC ATCAACACCT |
| 9061 | CTTGAGATAT CGAGCATCTT ACCGCCATTT ATACCCATAT TTGTTCTGTT TTTCTTGATT |
| 9121 | TGGGTATACA TTTAAATGTT AATAAAACAA AATGGTGGGG CAATCATTTA CATTTTTAGG |
| 9181 | GATATGTAAT TACTAGTTCA GGTGTATTGC CACAAGACAA ACATGTTAAG AAACTTTCCC |
| 9241 | GTTATTTACG CTCTGTTCCT GTTAATCAAC CTCTGGATTA CAAAATTTGT GAAAGATTGA |
| 9301 | CTGATATTCT TAACTATGTT GCTCCTTTTA CGCTGTGTGG ATATGCTGCT TTATAGCCTC |
| 9361 | TGTATCTAGC TATTGCTTCC CGTACGGCTT TCGTTTTCTC CTCCTTGTAT AAATCCTGGT |
| 9421 | TGCTGTCTCT TTTAGAGGAG TTGTGGCCCG TTGTCCGTCA ACGTGGCGTG GTGTGCTCTG |
| 9481 | TGTTTGCTGA CGCAACCCCC ACTGGCTGGG GCATTGCCAC CACCTGTCAA CTCCTTTCTG |
| 9541 | GGACTTTCGC TTTCCCCCTC CCGATCGCCA CGGCAGAACT CATCGCCGCC TGCCTTGCCC |
| 9601 | GCTGCTGGAC AGGGGCTAGG TTGCTGGGCA CTGATAATTC CGTGGTGTTG TCAGTACTGG |
| 9661 | TACCTTTAAG ACCAATGACT TACAAGGCAG CTGTAGATCT TAGCCACTTT TTAAAAGAAA |
| 9721 | AGGGGGGACT GGAAGGGCTA ATTCACTCCC AAAGAAGACA AGATCTGCTT TTTGCCTGTA |
| 9781 | CTGGGTCTCT CTGGTTAGAC CAGATCTGAG CCTGGGAGCT CTCTGGCTAA CTAGGGAACC |
| 9841 | CACTGCTTAA GCCTCAATAA AGCTTGCCTT GAGTGCTTCA A |

CD19_T2AW_IL15 (SEQ ID NO: 31)

| | |
|---|---|
| 1 | MLLLVTSLLL CELPHPAFLL IPDIQMTQTT SSLSASLGDR VTISCRASQD ISKYLNWYQQ |
| 61 | KPDGTVKLLI YHTSRLHSGV PSRFSGSGSG TDYSLTISNL EQEDIATYFC QQGNTLPYTF |
| 121 | GGGTKLEITG STSGSGKPGS GEGSTKGEVK LQESGPGLVA PSQSLSVTCT VSGVSLPDYG |
| 181 | VSWIRQPPRK GLEWLGVIWG SETTYYNSAL KSRLTIIKDN SKSQVFLKMN SLQTDDTAIY |
| 241 | YCAKHYYYGG SYAMDYWGQG TSVTVSSAAA GGGGSGGGGS GGGGSLEDGN EEMGGITQTP |
| 301 | YKVSISGTTV ILTCPQYPGS EILWQHNDKN IGGDEDDKNI GSDEDHLSLK EFSELEQSGY |
| 361 | YVCYPRGSKP EDANFYLYLR ARVCENCMEM DVMSVATIVI VDICITGGLL LLVYYWSKNR |
| 421 | KAKAKPVTRG AGAGGRQRGQ NKERPPPVPN PDYEPIRKGQ RDLYSGLNQR RIGSGEGRGS |
| 481 | LLTCGDVEEN PGPMRISKPH LRSISIQCYL CLLLNSHFLT EAGIHVFILG CFSAGLPKTE |
| 541 | ANWVNVISDL KKIEDLIQSM HIDATLYTES DVHPSCKVTA MKCFLLELQV ISLESGDASI |
| 601 | HDTVENLIIL ANNSLSSNGN VTESGCKECE ELEEKNIKEF LQSFVHIVQM FINTS |

MSLN_T2AW_IL15 (SEQ ID NO: 32)

| | |
|---|---|
| 1 | MLLLVTSLLL CELPHPAFLL IPEVQLVESG GGLVQPGGSL RLSCAASGGD WSANFMYWYR |
| 61 | QAPGKQRELV ARISGRGVVD YVESVKGRFT ISRDNSKNTL YLQMNSLRAE DTAVYYCAVA |
| 121 | SYWGQGTLVT VSSAAAGGGG SGGGGSGGGG SLEDGNEEMG GITQTPYKVS ISGTTVILTC |
| 181 | PQYPGSEILW QHNDKNIGGD EDDKNIGSDE DHLSLKEFSE LEQSGYYVCY PRGSKPEDAN |
| 241 | FYLYLRARVC ENCMEMDVMS VATIVIVDIC ITGGLLLLVY YWSKNRKAKA KPVTRGAGAG |
| 301 | GRQRGQNKER PPPVPNPDYE PIRKGQRDLY SGLNQRRIGS GEGRGSLLTC GDVEENPGPM |
| 361 | RISKPHLRSI SIQCYLCLLL NSHFLTEAGI HVFILGCFSA GLPKTEANWV NVISDLKKIE |
| 421 | DLIQSMHIDA TLYTESDVHP SCKVTAMKCF LLELQVISLE SGDASIHDTV ENLIILANNS |
| 481 | LSSNGNVTES GCKECEELEE KNIKEFLQSF VHIVQMFINT S |

CD19 TFP (SEQ ID NO: 33)

| | |
|---|---|
| 1 | MLLLVTSLLL CELPHPAFLL IPDIQMTQTT SSLSASLGDR VTISCRASQD ISKYLNWYQQ |
| 61 | KPDGTVKLLI YHTSRLHSGV PSRFSGSGSG TDYSLTISNL EQEDIATYFC QQGNTLPYTF |
| 121 | GGGTKLEITG GGGSGGGGSG GGGSEVKLQE SGPGLVAPSQ SLSVTCTVSG VSLPDYGVSW |
| 181 | IRQPPRKGLE WLGVIWGSET TYYNSALKSR LTIIKDNSKS QVFLKMNSLQ TDDTAIYYCA |
| 241 | KHYYYGGSYA MDYWGQGTSV TVSSAAAGGG GSGGGGSGGG GSLEDGNEEM GGITQTPYKV |
| 301 | SISGTTVILT CPQYPGSEIL WQHNDKNIGG DEDDKNIGSD EDHLSLKEFS ELEQSGYYVC |
| 361 | YPRGSKPEDA NFYLYLRARV CENCMEMDVM SVATIVIVDI CITGGLLLLV YYWSKNRKAK |
| 421 | AKPVTRGAGA GGRQRGQNKE RPPPVPNPDY EPIRKGQRDL YSGLNQRRI |

MSLN TFP (SEQ ID NO: 34)

| | |
|---|---|
| 1 | MLLLVTSLLL CELPHPAFLL IPEVQLVESG GGLVQPGGSL RLSCAASGGD WSANFMYWYR |
| 61 | QAPGKQRELV ARISGRGVVD YVESVKGRFT ISRDNSKNTL YLQMNSLRAE DTAVYYCAVA |
| 121 | SYWGQGTLVT VSSAAAGGGG SGGGGSGGGG SLEDGNEEMG GITQTPYKVS ISGTTVILTC |
| 181 | PQYPGSEILW QHNDKNIGGD EDDKNIGSDE DHLSLKEFSE LEQSGYYVCY PRGSKPEDAN |
| 241 | FYLYLRARVC ENCMEMDVMS VATIVIVDIC ITGGLLLLVY YWSKNRKAKA KPVTRGAGAG |
| 301 | GRQRGQNKER PPPVPNPDYE PIRKGQRDLY SGLNQRRI |

TABLE 1
Anti-CD70 binding domain sequences

| SEQ | | |
|---|---|---|
| ID | | |
| NO: | Name | Sequence |
| 45 | CD70 CDR1 | SIFDIVRGS |
| 46 | CD70 CDR2 | AIITSGGATNYA |
| 47 | CD70 CDR3 | CNMESVRYRNYW |
| 48 | CD70 variable | QVQLQESGGGLVQTGGSLRLSCTASGSIFDIVRGSWYRQAPG |
| | domain | NQRELVAIITSGGATNYADSVAGRFTISRDSAWKALYLQMNS |
| | | LKPEDTAVYFCNMESVRYRNYWGQGTQVTVSS |
| 49 | CD70 CDR1 | FTLEHYSIG |
| 50 | CD70 CDR2 | SCITSSGGIPKYA |
| 51 | CD70 CDR3 | CGAATPDDDCSVPGHYGLNYW |
| 52 | CD70 variable | QVQLQESGGGLVQPGGSLRLSCAASGFTLEHYSIGWFRQAPG |
| | domain | KDLEGVSCITSSGGIPKYADSVKGRFIISRDNAKNTGYLQMNS |
| | | LKPEDTAVYYCGAATPDDDCSVPGHYGLNYWGKGTQVTVS |
| | | S |
| 53 | CD70 CDR1 | FTFDAYAIG |
| 54 | CD70 CDR2 | ICLSPSDGSTYYA |
| 55 | CD70 CDR3 | CATPSWCSLKADFGSW |
| 56 | CD70 variable | QVQLQESGGGLVQAGGSLRLSCAAPGFTFDAYAIGWFRQAP |
| | domain | GKEREGVICLSPSDGSTYYADSVKGRFTISSDNAKNTVYLQM |
| | | NSLKPEDTAVYYCATPSWCSLKADFGSWGQGTQVTVSS |
| 57 | CD70 CDR1 | SIFSATRME |
| 58 | CD70 CDR2 | AIVTSGGRTNYA |
| 59 | CD70 CDR3 | CKFERYDYVNYW |
| 60 | CD70 variable | QVQLQESGGGLVQPGGSLRLSCTASGSIFSATRMEWYRQAP |
| | domain | GKQRELVAIVTSGGRTNYADSVNGRFTISRDNAKNTLYLQM |
| | | NNLKPEDTAVYYCKFERYDYVNYWGRGTQVTVSS |
| 61 | CD70 CDR1 | SIFSIARMN |
| 62 | CD70 CDR2 | AILNRAGRTDYA |
| 63 | CD70 CDR3 | CNLQTISYHDFW |
| 64 | CD70 variable | QVQLQESGGGLVQPGGSLRLSCVASGSIFSIARMNWYRQAPG |
| | domain | KQRELVAILNRAGRTDYADSVKGRFTISSDNAKTTVYLQMN |
| | | SLKPEDTALYYCNLQTISYHDFWGQGTQVTVSS |
| 65 | CD70 CDR1 | SIFDIARGN |
| 66 | CD70 CDR2 | AIITSGGATNYA |
| 67 | CD70 CDR3 | CNMESLSYRHYW |
| 68 | CD70 variable | QVQLQESGGGLVQTGGSLRLSCTASGSIFDIARGNWYRQAPG |
| | domain | KQRELVAIITSGGATNYADSVAGRFTISRDDAKNTVYLQMN |
| | | GLKPEDTAVYFCNMESLSYRHYWGQGTQVTVSS |
| 69 | CD70 CDR1 | SIIRDNVMA |
| 70 | CD70 CDR2 | AIINTGGSANVD |
| 71 | CD70 CDR3 | CNVYYRDLW |
| 72 | CD70 variable | QVQLQESGGGLVQAGGSLRLSCAASKSIIRDNVMAWHRQAP |
| | domain | GKQRELVAIINTGGSANVDDSVKGRFTISRDNAKNMVYLQM |
| | | NNLKPEDTAVYYCNVYYRDLWGQGTQVTVSS |
| 73 | CD70 CDR1 | FTLDRYAVG |
| 74 | CD70 CDR2 | SCISSSGDIIKYA |
| 75 | CD70 CDR3 | CTAADPKDDCSVPGYYGLNYW |
| 76 | CD70 variable | QVQLQESGGGLVQPGGSLRLSCVASGFTLDRYAVGWFRQAP |
| | domain | GKELEGVSCISSSGDIIKYADSAKGRFTIARDNAKNTAYLQM |
| | | NSLKPEDTAVYYCTAADPKDDCSVPGYYGLNYWGKGTQVT |
| | | VSS |
| 77 | CD70 CDR1 | FTLDKYAIG |
| 78 | CD70 CDR2 | SCITSSSGVVKYA |
| 79 | CD70 CDR3 | CAAAGPPDDCSVPGYYGLNYW |
| 80 | CD70 | QVQLQESGGGLVQPGGSLRLSCVASGFTLDKYAIGWFRQAP |
| | domain | GKELEGVSCITSSSGVVKYADSVKGRFIISRDNTNNRAFLQMS |
| | | SLKPEDTAVYYCAAAGPPDDCSVPGYYGLNYWGKGTQVTV |
| | | SS |

TABLE 2
IL-15 construct sequences

| SEQ | | |
|---|---|---|
| ID | | |
| NO: | Name | Sequence |
| 81 | IL-15 | GIHVFILGCFSAGLPKTEANWVNVISDLKKIEDLIQSMHIDAT |
| | | LYTESDVHPSCKVTAMKCFLLELQVISLESGDASIHDTVENLII |
| | | LANNSLSSNGNVTESGCKECEELEEKNIKEFLQSFVHIVQMFI |
| | | NTS |
| 82 | MH1ϵ T2A | MLLLVTSLLLCELPHPAFLLIPEVQLVESGGGLVQPGGSLRLS |
| | mbIL-15Rα | CAASGGDWSANFMYWYRQAPGKQRELVARISGRGVVDYVE |
| | (IL-15-IL- | SVKGRFTISRDNSKNTLYLQMNSLRAEDTAVYYCAVASYWG |
| | 15RA fusion) | QGTLVTVSSAAAGGGGSGGGGSGGGGSLEDGNEEMGGITQT |
| | | PYKVSISGTTVILTCPQYPGSEILWQHNDKNIGGDEDDKNIGS |
| | | DEDHLSLKEFSELEQSGYYVCYPRGSKPEDANFYLYLRARVC |
| | | ENCMEMDVMSVATIVIVDICITGGLLLLVYYWSKNRKAKAK |
| | | PVTRGAGAGGRQRGQNKERPPPVPNPDYEPIRKGQRDLYSG |
| | | LNQRRIGSGEGRGSLLTCGDVEENPGPMRISKPHLRSISIQCYL |
| | | CLLLNSHFLTEAGIHVFILGCFSAGLPKTEANWVNVISDLKKI |
| | | EDLIQSMHIDATLYTESDVHPSCKVTAMKCFLLELQVISLESG |
| | | DASIHDTVENLIILANNSLSSNGNVTESGCKECEELEEKNIKEF |
| | | LQSFVHIVQMFINTSSGGGSGGGGSGGGGSGGGGSGGGSLQI |
| | | TCPPPMSVEHADIWVKSYSLYSRERYICNSGFKRKAGTSSLTE |
| | | CVLNKATNVAHWTTPSLKCIRDPALVHQRPAPPSTVTTAGVT |
| | | PQPESLSPSGKEPAASSPSSNNTAATTAAIVPGSQLMPSKSPST |
| | | GTTEISSHESSHGTPSQTTAKNWELTASASHQPPGVYPQGHS |
| | | DTTVAISTSTVLLCGLSAVSLLACYLKSRQTPPLASVEMEAM |
| | | EALPVTWGTSSRDEDLENCSHHLDYKDDDDKDYKDDDDKD |
| | | YKDDDDK |
| 83 | VIN10299 | MLLLVTSLLLCELPHPAFLLIPEVQLVESGGGLVQPGGSLRLS |
| | pLRPO MH1ϵ | CAASGGDWSANFMYWYRQAPGKQRELVARISGRGVVDYVE |
| | T2AW PD- | SVKGRFTISRDNSKNTLYLQMNSLRAEDTAVYYCAVASYWG |
| | 1(EC- | QGTLVTVSSAAAGGGGSGGGGSGGGGSLEDGNEEMGGITQT |
| | TM)CD28(IC) | PYKVSISGTTVILTCPQYPGSEILWQHNDKNIGGDEDDKNIGS |
| | IL-15Ra(IC) | DEDHLSLKEFSELEQSGYYVCYPRGSKPEDANFYLYLRARVC |
| | | ENCMEMDVMSVATIVIVDICITGGLLLLVYYWSKNRKAKAK |
| | | PVTRGAGAGGRQRGQNKERPPPVPNPDYEPIRKGQRDLYSG |
| | | LNQRRIGSGEGRGSLLTCGDVEENPGPGMQIPQAPWPVVWA |
| | | VLQLGWRPGWFLDSPDRPWNPPTFSPALLVVTEGDNATFTC |
| | | SFSNTSESFVLNWYRMSPSNQTDKLAAFPEDRSQPGQDCRFR |
| | | VTQLPNGRDFHMSVVRARRNDSGTYLCGAISLAPKAQIKESL |
| | | RAELRVTERRAEVPTAHPSPSPRPAGQFQTLVVGVVGGLLGS |
| | | LVLLVWVLAVIRSKRSRLLHSDYMNMTPRRPGPTRKHYQPY |
| | | APPRDFAAYRSKSRQTPPLASVEMEAMEALPVTWGTSSRDE |
| | | DLENCSHHL |
| 84 | IL-15-IL15Rα | GIHVFILGCFSAGLPKTEANWVNVISDLKKIEDLIQSMHIDAT |
| | fusion | LYTESDVHPSCKVTAMKCFLLELQVISLESGDASIHDTVENLII |
| | | LANNSLSSNGNVTESGCKECEELEEKNIKEFLQSFVHIVQMFI |
| | | NTSSGGGSGGGGSGGGGSGGGGSGGGSLQITCPPPMSVEHA |
| | | DIWVKSYSLYSRERYICNSGFKRKAGTSSLTECVLNKATNVA |
| | | HWTTPSLKCIRDPALVHQRPAPPSTVTTAGVTPQPESLSPSGK |
| | | EPAASSPSSNNTAATTAAIVPGSQLMPSKSPSTGTTEISSHESS |
| | | HGTPSQTTAKNWELTASASHQPPGVYPQGHSDTTVAISTSTV |
| | | LLCGLSAVSLLACYLKSRQTPPLASVEMEAMEALPVTWGTSS |
| | | RDEDLENCSHHL |
| 85 | PD-1-CD28- | PGWFLDSPDRPWNPPTFSPALLVVTEGDNATFTCSFSNTSESF |
| | IL-15Rα fusion | VLNWYRMSPSNQTDKLAAFPEDRSQPGQDCRFRVTQLPNGR |
| | | DFHMSVVRARRNDSGTYLCGAISLAPKAQIKESLRAELRVTE |
| | | RRAEVPTAHPSPSPRPAGQFQTLVVGVVGGLLGSLVLLVWV |
| | | LAVIRSKRSRLLHSDYMNMTPRRPGPTRKHYQPYAPPRDFAA |
| | | YRSKSRQTPPLASVEMEAMEALPVTWGTSSRDEDLENCSHH |
| | | L |
| 86 | PD-1-CD28 | PGWFLDSPDRPWNPPTFSPALLVVTEGDNATFTCSFSNTSESF |
| | fusion | VLNWYRMSPSNQTDKLAAFPEDRSQPGQDCRFRVTQLPNGR |
| | | DFHMSVVRARRNDSGTYLCGAISLAPKAQIKESLRAELRVTE |
| | | RRAEVPTAHPSPSPRPAGQFQTLVVGVVGGLLGSLVLLVWV |
| | | LAVIRSKRSRLLHSDYMNMTPRRPGPTRKHYQPYAPPRDFAA |
| | | YRS |
| 87 | hIL-15Rα | DPALVHQRPAPPSTVTTAGVTPQPESLSPSGKEPAASSPSSNN |
| | | TAATTAAIVPGSQLMPSKSPSTGTTEISSHESSHGTPSQTTAKN |
| | | WELTASASHQPPGVYPQGHSDTTVAISTSTVLLCGLSAVSLL |
| | | ACYLKSRQTPPLASVEMEAMEALPVTWGTSSRDEDLENCSH |
| | | HL |
| 88 | VIN70080 | MLLLVTSLLLCELPHPAFLLIPEVQLVESGGGLVQPGGSLRLS |
| | pLRPCMH1ϵ | CAASGGDWSANFMYWYRQAPGKQRELVARISGRGVVDYVE |
| | T2A PD-1(EC- | SVKGRFTISRDNSKNTLYLQMNSLRAEDTAVYYCAVASYWG |
| | TM)CD28(IC) | QGTLVTVSSAAAGGGGSGGGGSGGGGSLEDGNEEMGGITQT |
| | IL-15Ra(IC) | PYKVSISGTTVILTCPQYPGSEILWQHNDKNIGGDEDDKNIGS |
| | P2A IL-15 | DEDHLSLKEFSELEQSGYYVCYPRGSKPEDANFYLYLRARVC |
| | | ENCMEMDVMSVATIVIVDICITGGLLLLVYYWSKNRKAKAK |
| | | PVTRGAGAGGRQRGQNKERPPPVPNPDYEPIRKGQRDLYSG |
| | | LNQRRIGSGEGRGSLLTCGDVEENPGPGMQIPQAPWPVVWA |
| | | VLQLGWRPGWFLDSPDRPWNPPTFSPALLVVTEGDNATFTC |
| | | SFSNTSESFVLNWYRMSPSNQTDKLAAFPEDRSQPGQDCRFR |
| | | VTQLPNGRDFHMSVVRARRNDSGTYLCGAISLAPKAQIKESL |
| | | RAELRVTERRAEVPTAHPSPSPRPAGQFQTLVVGVVGGLLGS |
| | | LVLLVWVLAVIRSKRSRLLHSDYMNMTPRRPGPTRKHYQPY |
| | | APPRDFAAYRSKSRQTPPLASVEMEAMEALPVTWGTSSRDE |
| | | DLENCSHHLGSGATNFSLLKQAGDVEENPGPMRISKPHLRSIS |
| | | IQCYLCLLLNSHFLTEAGIHVFILGCFSAGLPKTEANWVNVIS |
| | | DLKKIEDLIQSMHIDATLYTESDVHPSCKVTAMKCFLLELQVI |
| | | SLESGDASIHDTVENLIILANNSLSSNGNVTESGCKECEELEEK |
| | | NIKEFLQSFVHIVQMFINT |
| 89 | VIN11023_pL | MLLLVTSLLLCELPHPAFLLIPEVQLVESGGGLVQPGGSLRLS |
| | KaUS MH1ϵ | CAASGGDWSANFMYWYRQAPGKQRELVARISGRGVVDYVE |
| | T2AW | SVKGRFTISRDNSKNTLYLQMNSLRAEDTAVYYCAVASYWG |
| | solubleIL- | QGTLVTVSSAAAGGGGSGGGGSGGGGSLEDGNEEMGGITQT |
| | 15Rα | PYKVSISGTTVILTCPQYPGSEILWQHNDKNIGGDEDDKNIGS |
| | | DEDHLSLKEFSELEQSGYYVCYPRGSKPEDANFYLYLRARVC |
| | | ENCMEMDVMSVATIVIVDICITGGLLLLVYYWSKNRKAKAK |
| | | PVTRGAGAGGRQRGQNKERPPPVPNPDYEPIRKGQRDLYSG |
| | | LNQRRIGSGEGRGSLLTCGDVEENPGPMRISKPHLRSISIQCYL |
| | | CLLLNSHFLTEAGIHVFILGCFSAGLPKTEANWVNVISDLKKI |
| | | EDLIQSMHIDATLYTESDVHPSCKVTAMKCFLLELQVISLESG |
| | | DASIHDTVENLIILANNSLSSNGNVTESGCKECEELEEKNIKEF |
| | | LQSFVHIVQMFINTSSGGGSGGGGSGGGGSGGGGSGGGSLQI |
| | | TCPPPMSVEHADIWVKSYSLYSRERYICNSGFKRKAGTSSLTE |
| | | CVLNKATNVAHWTTPSLKCIRDPALVHQRPAPPSTVTTAGVT |
| | | PQPESLSPSGKEPAASSPSSNNTAATTAAIVPGSQLMPSKSPST |
| | | GTTEISSHESSHGTPSQTTAKNWELTASASHQPPGVYPQGHS |
| | | DTT |
| 90 | MH1e T2Aw | MLLLVTSLLLCELPHPAFLLIPEVQLVESGGGLVQPGGSLRLS |
| | IL-15 | CAASGGDWSANFMYWYRQAPGKQRELVARISGRGVVDYVE |
| | | SVKGRFTISRDNSKNTLYLQMNSLRAEDTAVYYCAVASYWG |
| | | QGTLVTVSSAAAGGGGSGGGGSGGGGSLEDGNEEMGGITQT |
| | | PYKVSISGTTVILTCPQYPGSEILWQHNDKNIGGDEDDKNIGS |
| | | DEDHLSLKEFSELEQSGYYVCYPRGSKPEDANFYLYLRARVC |
| | | ENCMEMDVMSVATIVIVDICITGGLLLLVYYWSKNRKAKAK |
| | | PVTRGAGAGGRQRGQNKERPPPVPNPDYEPIRKGQRDLYSG |
| | | LNQRRIGSGEGRGSLLTCGDVEENPGPMRISKPHLRSISIQCYL |
| | | CLLLNSHFLTEAGIHVFILGCFSAGLPKTEANWVNVISDLKKI |
| | | EDLIQSMHIDATLYTESDVHPSCKVTAMKCFLLELQVISLESG |
| | | DASIHDTVENLIILANNSLSSNGNVTESGCKECEELEEKNIKEF |
| | | LQSFVHIVQMFINTS |

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Example 1: Three Methods for Preparation of γδ T-Cells Transduced with TFPs

Lentiviral Production

Lentivirus encoding the TFPs are prepared by transfecting Expi293F cells with plasmids-PEIPro mixture. Third generation HIV-based lentiviral vector system can be used to make lentivirus. Plasmid encoding different components, pTransfer (transgene of interest), pGAGPOL, pRSVcoREV, pCMV-VSV-G-bGpA, can be mixed with PEIPro (Polyplus) at optimized ratios and added to Expi293F cells (Day 1). Medium change can be performed on day 2 to remove the transfection complex from the cells. Cells can be spun down at 500×g for 10 minutes and resuspended in medium supplemented with sodium butyrate. The lentivirus can be harvested on day 3 by collecting the culture supernatant with centrifugation of the culture at 3000×g for 30 minutes. The supernatant can be filtered through 0.45 μm PES filter and then concentrated by adding 1 volume of cold Lenti-X virus precipitation solution (Takara Clontech) to every 3 volumes of lentivirus-containing supernatant. The lentivirus can be precipitated at 48-96 hours afterwards. After spinning at 3000×g for 60 minutes, the supernatant can be discarded and the pellet can be resuspended with desired formulation buffer, aliquoted as desired and stored at −80° C.

PBMC Isolation

Peripheral blood mononuclear cells (PBMCs) are prepared from leukopak, whole blood or buffy coat that obtained from healthy donors via commercial resources. Leukopak, whole blood or buffy coat are diluted with sterile phosphate buffered saline (PBS, pH 7.4, without Ca²⁺/Mg²⁺). PBMCs will be separated by isolation protocol using Ficoll-Paque® PLUS (GE Healthcare, 17-1440-03) in 50 mL conical centrifuge tubes with centrifugation at 400 g for 30-40 min at room temperature with no brake application.

Approach 1: Expand and Activate PBMCs

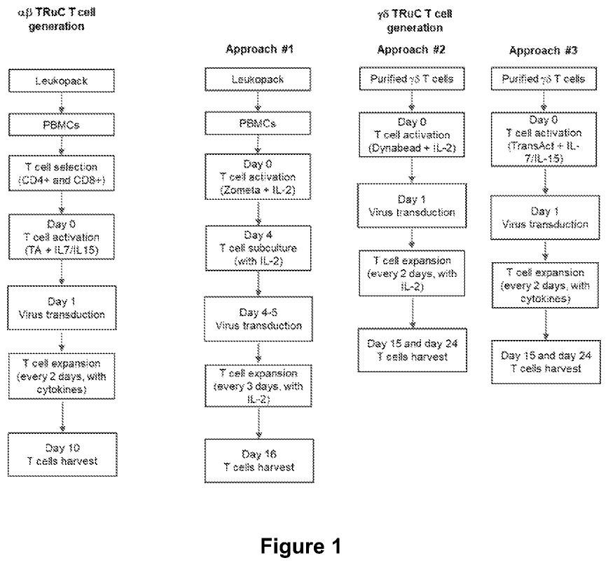

As is shown in FIG. 1, three different protocols are used for activating and expanding γδ T cells. In the first approach, TFP-expressing γδ T cells are generated from PBMCs with the treatment of Zometa (Sigma, SML0223) and recombinant human IL-2 (Peprotech, 200-02). PBMCs (diluted in RPMI-1640 medium supplemented with 10% HI-FBS at 10⁶ cells/mL) can be treated with Zometa at 5 μM and IL-2 at 100 IU/mL (considered as day 0). Treated cells can be washed at day 4 and sub-cultured in medium with IL-2 (100 IU/mL). In certain instance, lentivirus encoding the TFP can be used to transduce the treated cells on day 4, day 5 or day 4 and 5. In other instance, mRNA encoding the TFP can be electroporated to the treated cells at desired day of the process. The cells can then be sub-cultured on day 9 and day 14 with medium with IL-2 (100 IU/mL). Cell concentration can be adjusted to 10⁶ cells/mL at each subculture.

In approaches 2 and 3, γδ T cells are first isolated from PBMC by negative selection with the TCRγ/δ+ T Cell Isolation Kit, human (Miltenyi Biotech, 130-092-892). In approach 2, purified γδ T cells can be activated by Dynabeads™ Human T-Activator CD3/CD28 for T Cell Expansion and Activation (ThermoFisher Scientific, 11132D) and recombinant human IL-2 (Peprotech, 200-02). Purified γδ T cells can be diluted at 10⁶ cells/mL with AIM V-AlbuMAX™ (BSA) (Life Technologies), supplemented with 5% human AB serum (Gemini Bioproducts, 100-318) and 300 IU/mL IL-2. T cells activation can be done with adding Dynabeads to the cells at 1:1 ratio (considered as day 0). Lentivirus encoding the TFP can be used to transduce the γδ T cells on day 1. The cells will be washed on day 4 and sub-cultured with the medium and IL-2 (300 IU/mL). Then the cells can be sub-cultured every 2 days with the medium and IL-2 (300 IU/mL). Cell concentration can be adjusted to 5×10⁵ cells/mL at each subculture. In approach 3, purified γδ T cells can be activated by MACS GMP T cell TransAct, for Research Use (Miltenyi Biotech, 130-019-011) and MACS GMP Recombinant human IL-7 (Miltenyi Biotech, 170-076-11) and MACS GMP Recombinant human IL-15 (Miltenyi Biotech, 170-076-114). Purified γδ T cells can be diluted at 10⁶ cells/mL with TexMACS™ GMP Medium (Miltenyi Biotech, 170-076-307), supplemented with 3% human AB serum (Gemini Bioproducts, 100-318) and 12.5 ng/mL IL-7 and 12.5 ng/mL IL-15. T cells activation can be done with adding TransAct to the cells at 1:1 ratio (considered as day 0). Lentivirus encoding the TFP can be used to transduce the γδ T cells on day 1. The cells will be washed on day 4 and sub-cultured with the medium and 12.5 ng/mL IL-7 and 12.5 ng/mL IL-15. Then the cells can be sub-cultured every 2 days with the medium and 12.5 ng/mL IL-7 and 12.5 ng/mL IL-15. Cell concentration can be adjusted to 5×10⁵ cells/mL at each subculture.

T-Cell Transduction/Transfection

Lentivirus is thawed on ice and 5×10⁶ lentivirus, along with 2 μL of TransPlus™ (Alstem) per mL of media (a final dilution of 1:500) is added to each well of 1×10⁶ cells.

Alternatively, lentivirus is thawed on ice and the respective virus is added at 5 or 50 MOI in presence of 5 μg/mL polybrene (Sigma). Cells are spinoculated at 100 g for 100 minutes at room temperature. Alternatively, cells are treated with LentiBoost before addition of lentivirus. Alternatively, transduction is repeated 24 hours afterwards. After transduction, cell concentrations are analyzed every 2-3 days, with media being added at that time to maintain the cell suspension at 1×10⁶ cells/mL.

In some instances, activated PBMCs or γδ T cells are electroporated with in vitro transcribed (IVT) mRNA. The cells are washed and suspended in OPTI-MEM medium (ThermoFisher) at the concentration of 2.5×10⁷ cells/mL. 200 μL of the cell suspension (5×10⁶ cells) are transferred to the 2 mm gap Electroporation Cuvettes Plus™ (Harvard Apparatus BTX) and prechilled on ice. 10 μg of IVT TFP mRNA is added to the cell suspension. The mRNA/cell mixture is then electroporated at 200 V for 20 milliseconds using ECM830 Electro Square Wave Porator (Harvard Apparatus BTX). Immediately after the electroporation, the cells are transferred to fresh cell culture medium (AIM V AlbuMAX (BSA) serum free medium+5% human AB serum+300 IU/ml IL-2) and incubated at 37° C.

Verification of TFP Expression by Cell Staining

Following lentiviral transduction or mRNA electroporation, expression of TFPs is confirmed by flow cytometry, with MonoRab™ Rabbit Anti-Camelid VHH Antibody [iFluor488] (GenScript, clone 96A3F5) for MSLN-specific TFP, or FTC-labelled human CD19 (20-291) (Acro Biosystems, CD9-HF251) for CD19-specific TFP.

During the generation of γδ T cells expressing TFPs, samples can be obtained at certain time points (day 0, day 4, day 7, day 12, day 15, day 24 for instance) for the characterization of the γδ T cell subsets and differentiation. Abs panel for this purpose can include: anti-human CD3 (clone UCHT1), anti-human TCR Vδ1 (clone REA173), anti-human TCR Vδ2 (clone B6), anti-human αβ TCR (clone IP26), anti-human pan γδ TCR (clone IMMU510), anti-human CD45 RA (clone H1100), anti-human CD27 (clone 0323).

### Example 2: Activation as Measured by CD69 or CD25 Upregulation on T Cells

Activation of the T-cells expressing TFP Constructs is measured. As described above, activated cells are transduced and expanded. Day 15 post transduction, cells are harvested and stimulated with plate-bound anti-human pan γδ TCR (clone IMMU510), anti-human CD3 (UCHT1), CD19-Fc (R&D System, 9269-CD-050) or MSLN-Fc (Acro Biosystems, MSN-H526x) for 24 hours. Expression of CD25 and CD69 are determined by flow cytometry with Ab panel including anti-human CD3 (clone UCHT1), anti-human TCR Vδ1 (clone REA173), anti-human TCR Vδ2 (clone B6), anti-human CD25 (Clone BC96) and anti-human CD69 (clone FN50).

Activation of T cells may be similarly assessed by analysis of granzyme B production. T cells are cultured and expanded as described above, and intracellular staining for granzyme B is done according to the manufacturer's kit instructions (Gemini Bioproducts; 100-318). Cells are harvested, washed with PBS three times and blocked with human Fc block for 10 min. Cells are stained for surface antigens with anti-CD69-Alexa Fluor® 700 (clone FN50) from BD Biosciences and anti-CD25-PE (Clone BC96, eBioscience) for 30 min at 4° C. Cells are then fixed with Fixation/Permeabilization solution (BD Cytofix/Cytoperm Fixation/Permealbilzation kit cat #554714) for 20 min at 4 C, flowed by washing with BD Perm/Wash buffer. Cells are subsequently stained with anti-Granzyme B Alexafluor700 (Clone GB11), washed with BD Perm/Wash buffer twice and resuspended in FACS buffer. Data is acquired on BD LSRII-Fortessa and analyzed using FlowJo® (Tree star Inc.).

### Example 3: Cytokine Secretion Measurement by MSD

A measure of effector T-cell activation and proliferation associated with the recognition of cells bearing cognate antigen is the production of effector cytokines such as interleukin-2 (IL-2) and interferon-gamma (IFN-γ), granulocyte-macrophage colony-stimulating factor (GM-CSF) and tumor necrosis factor alpha (TNF-α).

Target-specific cytokine production including IL-2, IFN-γ, GM-CSF, and TNF-α by monospecific TFP T cells is measured from supernatants harvested 48 hours after the co-culture of T cells with various tumor cells using the U-PLEX® Biomarker Group I (hu) Assays (Meso Scale Diagnostics®, LLC, catalog number: K15067L-4) or after culture of TFP containing cells with plate-bound anti-CD3ε, anti-pan γδ TCR, CD19-Fc, MSLN-Fc or medium alone.

### Example 4: Luciferase-Based Cytotoxicity Assay

The luciferase-based cytotoxicity assay assesses the cytotoxicity of TFP T cells by indirectly measuring the luciferase enzymatic activity in the residual live target cells after co-culture. Human tumor cell line K562 based target cells, expressing no target or MSLN are generated by transduction with lentivirus encoding the human MSLN. Target cells stably expressing desired target antigens are selected by application of antibiotics matched to the resistance gene encoded by the lentivirus. The target cells are further modified to overexpress firefly luciferase via transduction with firefly luciferase encoding lentivirus followed with antibiotic selection to generate stable cell line.

In a typical cytotoxicity assay, the target cells are plated at 10000 cells per well in 96-well plate. The TFP T or control cells are added to the target cells at different effector-to-target ratios. The mixture of cells are then cultured for 24 or 48 hours at 37° C. with 5% CO₂ before the luciferase enzymatic activity in the live target cells is measured by the Bright-Glo® Luciferase Assay System (Promega®, Catalogue number E2610). The cells are spun into a pellet and resuspended in medium containing the luciferase substrate. The percentage of tumor cell killing is then calculated with the following formula: % Cytotoxicity=100%×[1−RLU (Tumor cells+T cells)/RLU (Tumor cells)].

### Example 5: Allogeneic TFP Expressing Cells Prepared According to Approach 1

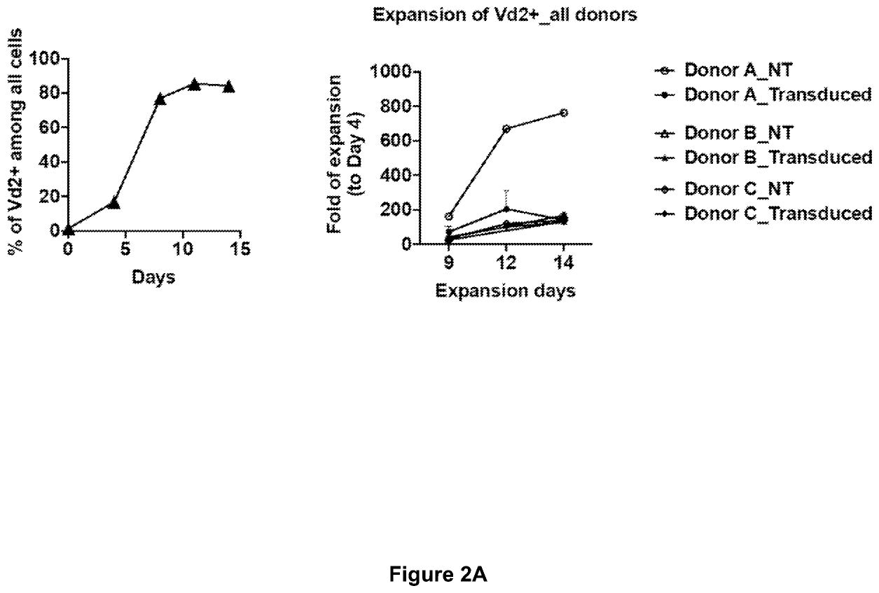

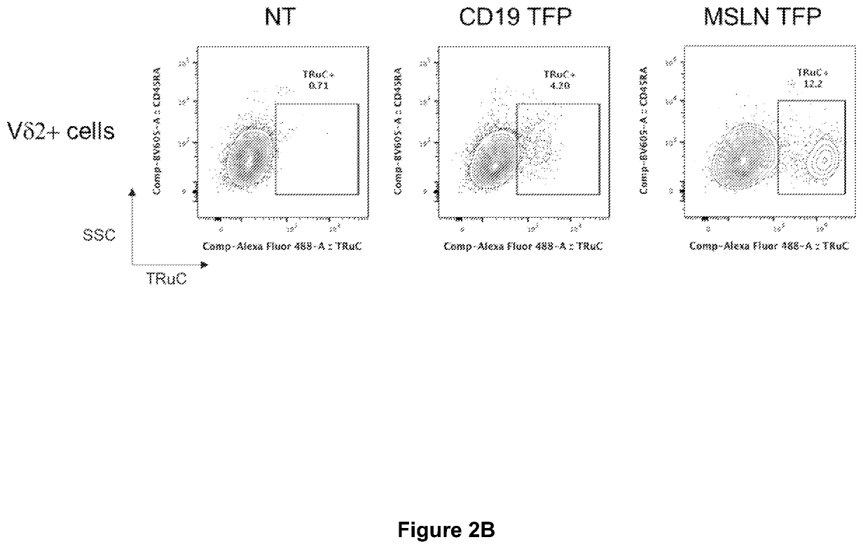

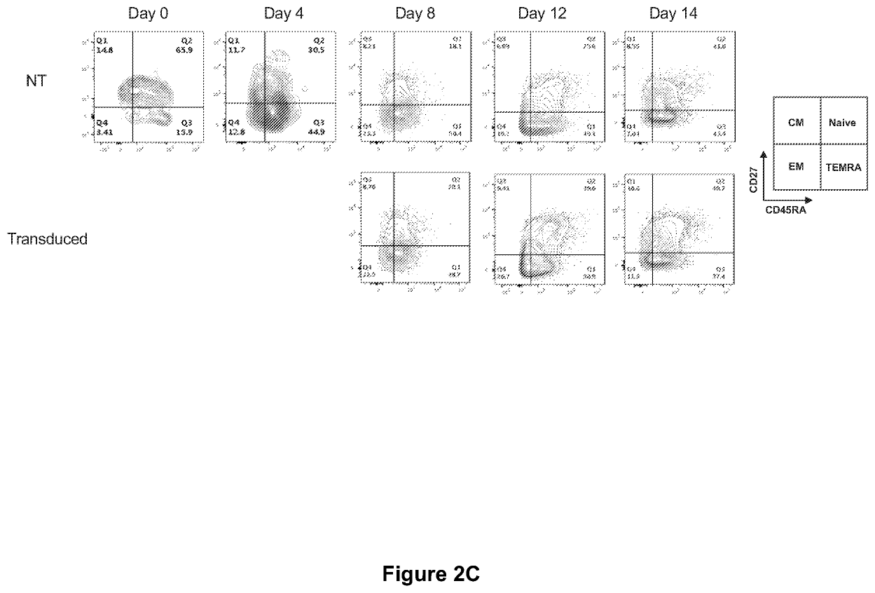

γδ T cells from three donors were transduced with lentivirus encoding TFPs according to the methods described in Example 1, Approach 1. As is described in Example 1, PBMCs were activated with IL-2 and zometa, transduced after 4-5 days with lentiviral vectors encoding a CD19 TFP described in WO2016187349 or a MSLN TFP described in WO2018067993, the entirety of each of which is incorporated by reference herein in their entirety. The cells were then expanded in the presence of IL2, and harvested on day 16. FIGS. 2A and 2B show the expansion of the Vδ2+ cells containing the TFP post transduction in all three donors as determined by flow cytometry using Ab panel described in Example 1. As shown in FIG. 2A (left panel), Vδ2+ T cells comprise a very small proportion of PBMCs. Approach 1 selectively expands Vδ2+ T cells over time. By day 7, more than 80% of the cultured cells are Vδ2+ T cells. The expansion of Vδ2+ cells are further determined for both non-transduced (NT) and transduced cells from each individual donor on day 9, 12 and 14, based on the number of total alive cells in culture and the percentage of Vδ2+ T cells. As shown in FIG. 2A (right panel), Vδ2+ cells from all 3 donors expanded more than 100-fold comparing to day 4, with certain level variation observed from donor to donor. FIG. 2B shows the TFP expression by Vδ2+ cells on day 12 from the representative donor. For cells transduced with CD19 TFP lentivirus, 4.2% of Vδ2+ cells are transduced. For cells transduced with MSLN TFP lentivirus, 12.2% of Vδ2+ cells are transduced. As shown in FIG. 2C, Vδ2+ cells are mainly naïve before the expansion (day 0) and gradually differentiate into effector memory (EM) and terminally differentiated effector (TEMRA) during expansion, regardless virus transduction status.

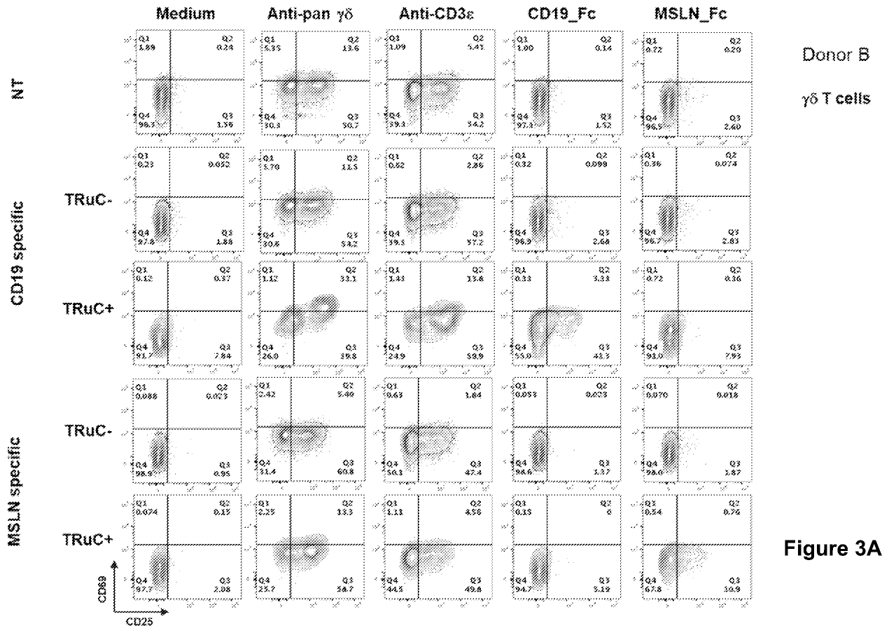

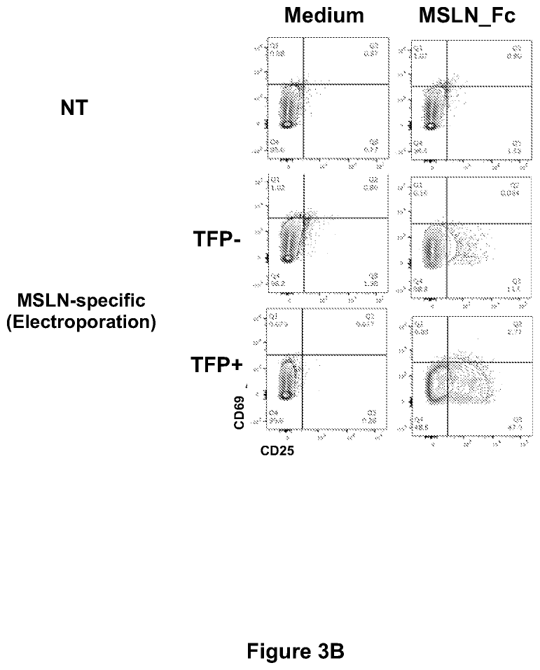

The activation of the cells was then measured as is described in Example 2 in Donors B and C. In particular, activation of cells treated with anti-CD3ε, anti-pan γδ TCR CD19-Fc, or MSLN-Fc, or an untreated control was measured by flow cytometry using anti-CD69-Alexa Fluor® 700 (clone FN50) from BD Biosciences and anti-CD25-PE as stains. Cells transduced with a lentiviral vector encoding MSLN and CD19 TFPs, cells electroporated with the MSLN TFP, and negative controls were treated. The results are shown in FIGS. 3A and 3B. FIG. 3A shows the ligand-specific induction of CD25 and CD69 expression from cells transduced by lentivirus. Stimulated cells are first gated on Vδ2+ population and then gated for TFP- and TFP+ populations. Medium treated cells serve as control for baseline expression of CD25 and CD69. All Vd2+ populations, with or without TFP expression, show robust response to stimulations via crosslinking TCR complex components such as γδ TCR, or CD3ε, indicating the expression of TFP does not jeopardize TCR signaling in Vδ2+ cells. The reactivity and specific of the TFPs are supported by the observations that CD19 TFP+ cells are activated only by CD19-Fc but not MSLN-Fc as well as the observation that MSLN TFP+ cells are activated by Fc_MSLN but not CD19_Fc. Alternatively, MSLN TFP-expressing cells are generated by electroporation of RNA to the cells on day 15 and treated with ligand immediately. TFP+Vd2+ cells generated by electroporation shows similarly specific and robust activation induced by MSLN-Fc as comparing to TFP+ cells generated by lentivirus (FIG. 3B).

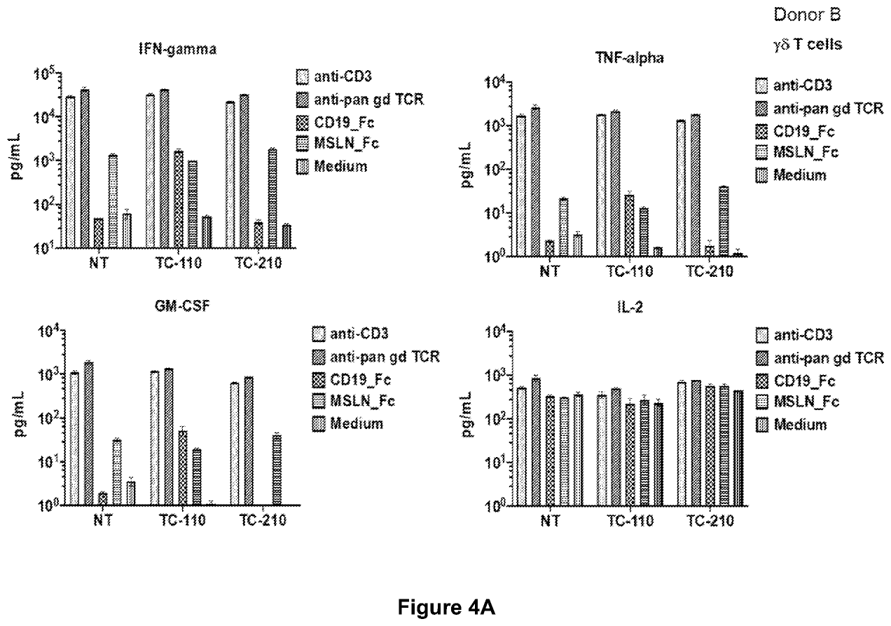

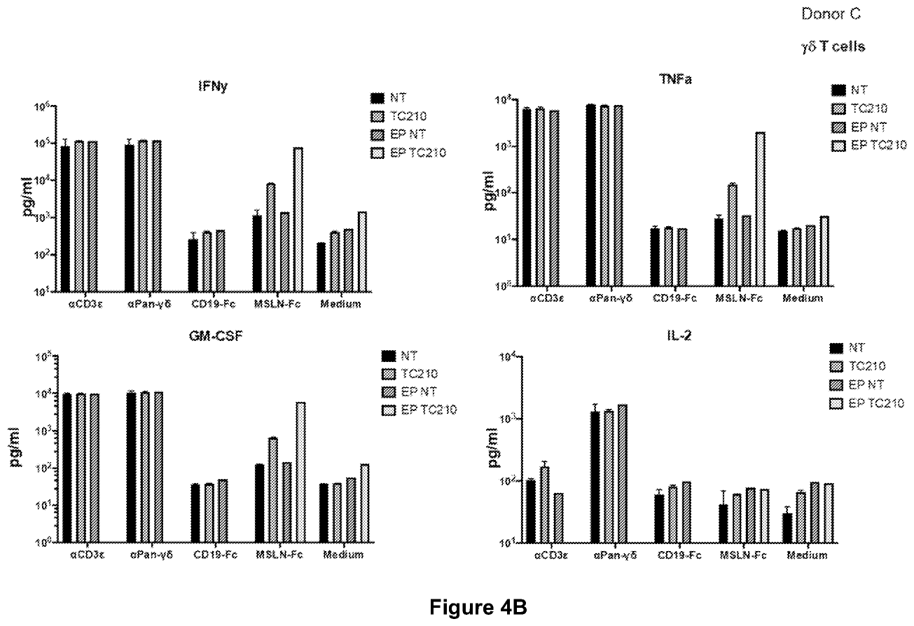

Cytokine production by the cells treated as in FIG. 3 is also measured in Donors B and C. Cells transduced with a lentiviral vector encoding MSLN and CD19 TFPs, cells electroporated with the MSLN TFP, and negative controls were treated with anti-CD3ε, anti-pan γδ, CD19-Fc, or MSLN-Fc, or media alone. Levels of IL-2, IFN-γ, GM-CSF, and TNF-α were measured as is described in Example 3. The results are shown in FIGS. 4A and 4B. For the donor shown in FIG. 4A, IL-2 is produced constitutively and not affected further by any stimulation. IFN-γ, TNF-α and GM-CSF are all induced by anti-γδ TCR and anti-CD3 stimulations. Furthermore, CD19 TFP cells, but not NT or MSLN TFP cells, react to CD19-Fc. All γδ T cells from this donor (NT, CD19 TFP or MSLN TFP) show similar level of cytokine production to MSLN-Fc stimulation, even though only MSLN-specific TFP+ cells demonstrate T cell activation in response to Fc_MSLN (FIG. 3A). FIG. 4B shows the cytokine detection for settings of FIG. 3B. Consistent with cell activation (FIG. 3B), electroporation generated MSLN-specific TFP cells produce significant amounts of IFN-γ, TNF-α and GM-CSF in response to Fc_MSLN (FIG. 4B). IL-2 is not being produced by the stimulated cells, even though the cells are capable of producing IL-2 in response to anti-pan γδ TCR. Consistent with cell activation results, TFP γδ T cells, generated by lentivirus (FIG. 4A) or electroporation (FIG. 4B) produce cytokines in response to TCR activation similarly as NT cells, and to ligands in a specific manner.

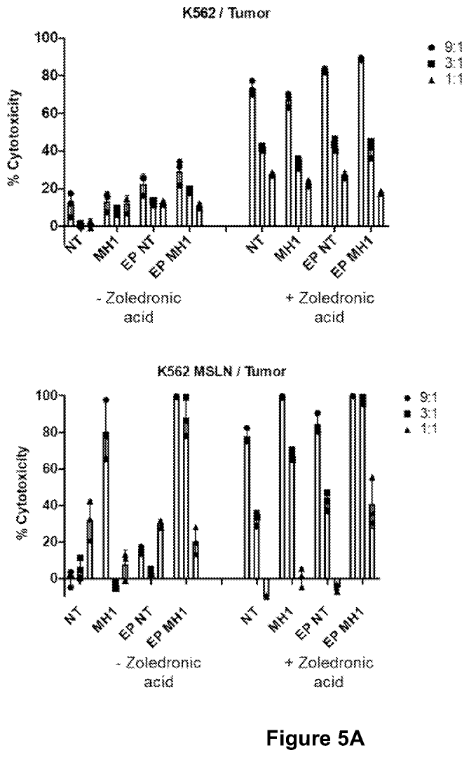

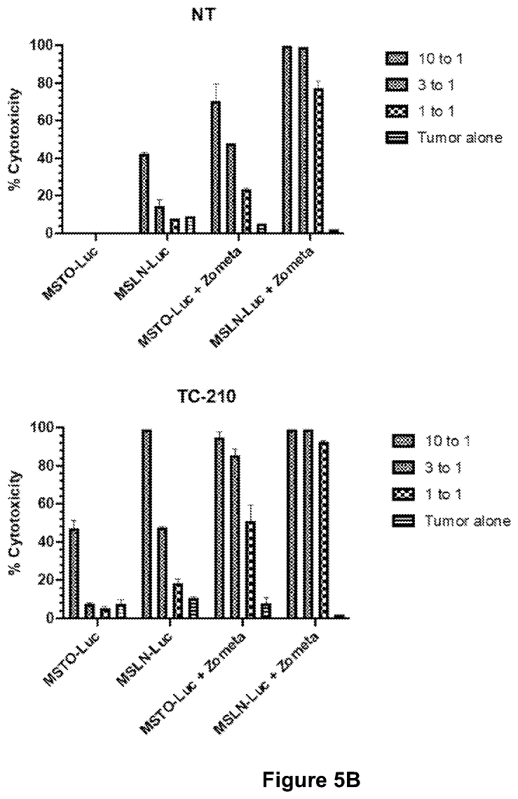

Cytotoxicity was also measured in MSLN TFP expressing cells from Donor C as is described in Example 4. As is shown in FIG. 5A, K562 target cells overexpressing firefly luciferase and expressing no target or MSLN were co-cultured with non-transduced (NT) cells or cells transduced with a lentiviral vector encoding MSLN TFP or electroporated with mRNA encoding MSLN TFP at the ratios shown. The cells were cultured in the presence or absence of Zoledronic acid, which increases surface presentation of the endogenous antigens targeted by the γδ T cell receptor in the K562 cells. FIG. 5B shows the same experiment done with a different donor with MSLN low MSTO-Luc cells and MSLNhi MSLN-Luc cells. The function of both MSLN TFP and Vd2 TCR are demonstrated by the cytotoxicity against MSLNhi cells (K562-MSLN and MSLN-Luc) in the absence of Zometa, and the cytotoxicity against MSLNlow cells (K562 and MSTO) in presence of Zometa, respectively.

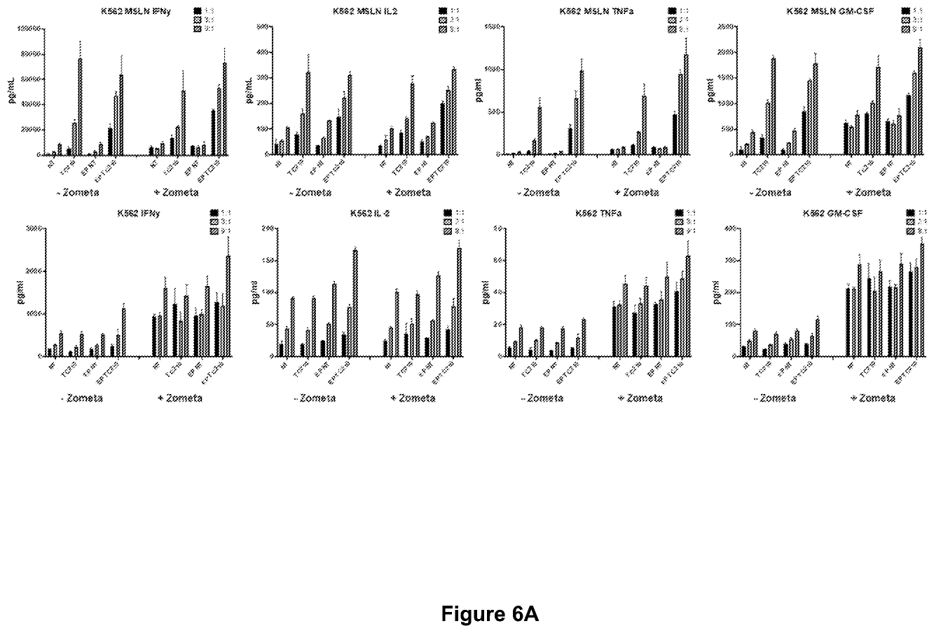

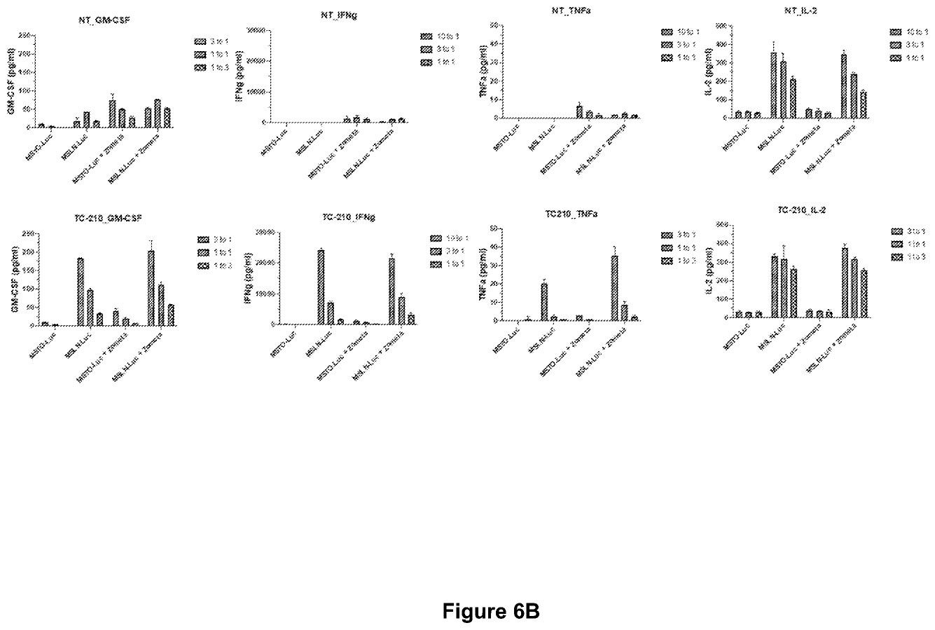

The induction of cytokine production by target cells was also measured as is described in Example 3. In FIG. 6A, K562 target cells expressing no target or MSLN were co-cultured with untransduced cells or cells transduced with a lentiviral vector encoding MSLN TFP or electroporated with mRNA encoding MSLN TFP at the ratios shown. The cells were cultured in the presence or absence of zometa. Levels of IL-2, IFN-γ, GM-CSF, and TNF-α were measured. FIG. 6B shows the same experiment done with with a different donor with MSLNlow MSTO-Luc cells and MSLNhi MSLN-Luc cells. Consistent with cytotoxicity results in FIGS. 5A and 5B, function of both MSLN TFP and Vδ2 TCR are demonstrated by the cytokine production against MSLNhi cells (K562-MSLN and MSLN-Luc) in the absence of Zometa, and the cytokine production against MSLNlow cells (K562 and MSTO) in presence of Zometa, respectively. The level of cytokine production from the same cells (MSLN TFP for instance) is always lower in Vδ2 TCR-activation (MSTO cells co-culture) than in MSLN TFP activation (MSLN cells co-culture) (FIG. 6B).

### Example 6: Allogeneic TFP Expressing Cells Prepared According to Approach 2

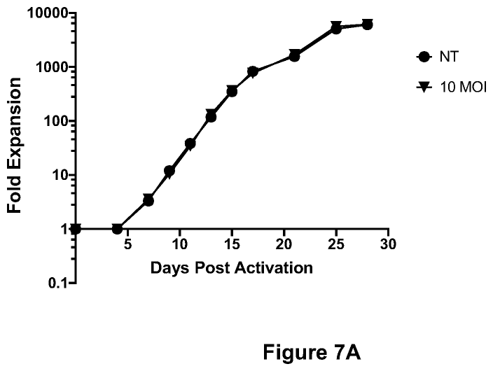

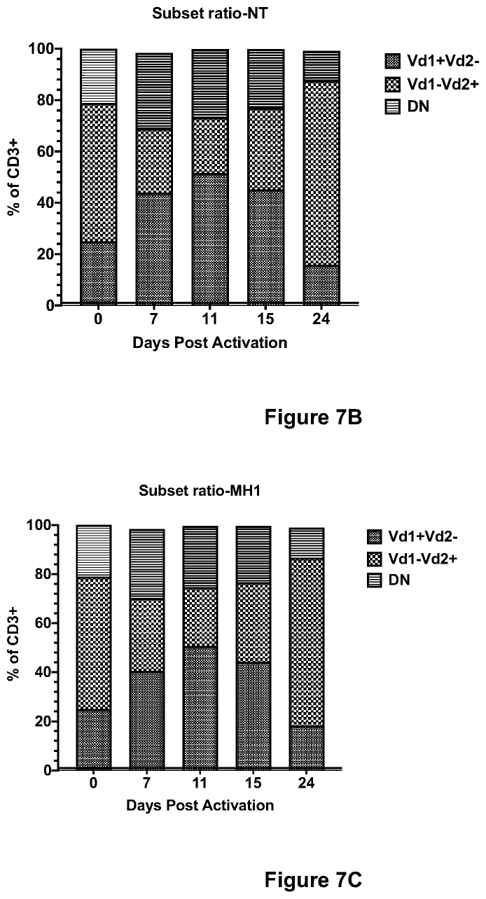

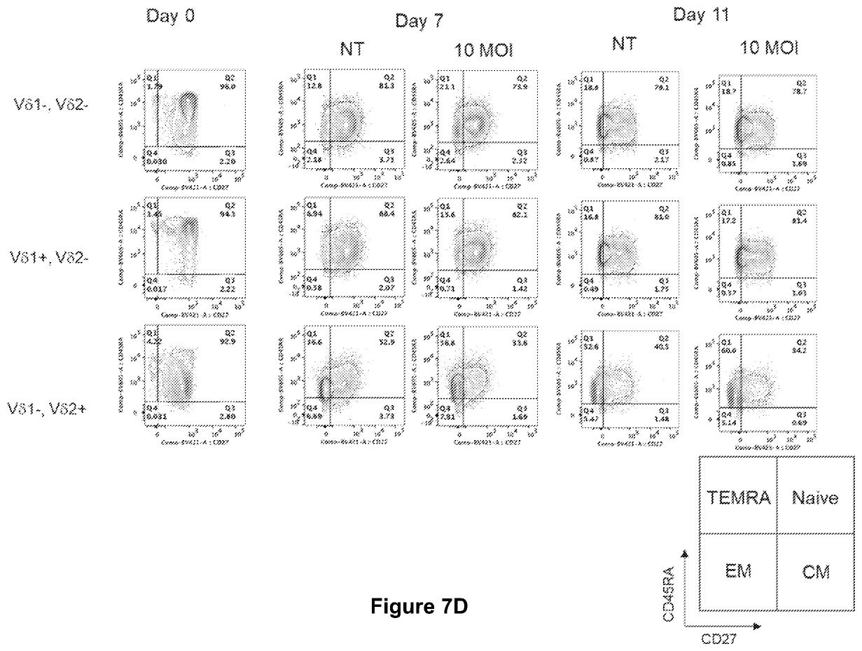

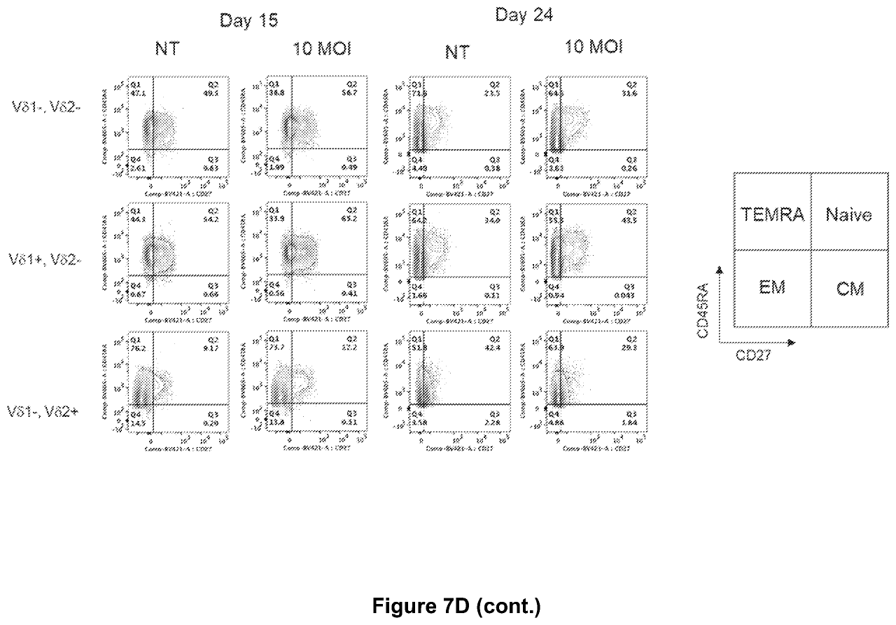

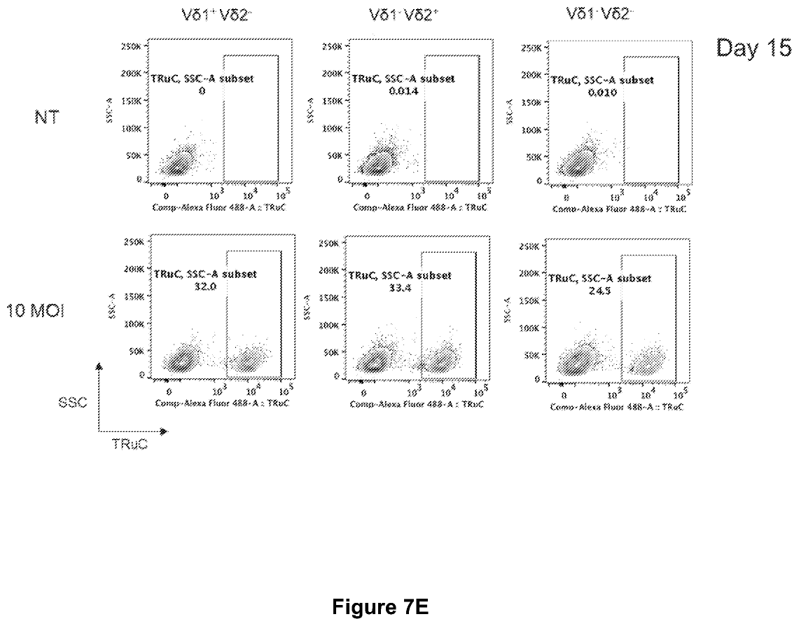

γδ T cells from one donor were transfected with a MSLN TFP according to the methods described in Example 1, Approach 2. As is described in Example 1, γδ T cells were isolated from PBMCs by negative selection. The cells were activated with IL-2 in the presence of anti-CD3 and anti-CD28-attached magnetic beads (Dynabeads), transduced after 1 day with a lentiviral vector encoding a MSLN TFP described above. The cells were then expanded in the presence of IL2, and harvested on days 15 and 24. FIG. 7A shows the expansion of the cells. FIGS. 7B and 7C show the proportion of Vδ1−Vδ2−, Vδ1+Vδ2−, and Vδ1−Vδ2+ cells at 7, 11, 15 and 24 days post-activation for non-transduced and transduced cells. FIG. 7D shows the differentiation state (whether the cell is naïve, central memory, effector memory, or TEMRA) as measured by anti-CD45RA and anti-CD27 flow cytometry for the Vδ1−Vδ2−, Vδ1+Vδ2−, and Vδ1−Vδ2+ subsets at 0, 7, 11, 15 and 24 days post-activation. FIG. 7E shows the for the Vδ1−Vδ2−, Vδ1+Vδ2−, and Vδ1−Vδ2+ subsets of untransduced and transduced cells as measured by flow cytometry at 15 days post-activation. Approach 2 demonstrates robust expansion of purified γδ T cells (more than 6000-fold) (FIG. 7A). In contrast to the Approach 1, which selectively expands Vδ2+ cells, Approach 2 expands all subsets with similar profiles for NT and

MSLN TFP transduced cells (FIGS. 7B and 7C). All subsets differentiate from naïve stage on day 0 towards TEMRA stage at day 24 (FIG. 7D). Transduction of Vδ1−Vδ2−, Vδ1+Vδ2−, and Vδ1−Vδ2+ subsets are detected at similar level (FIG. 7E).

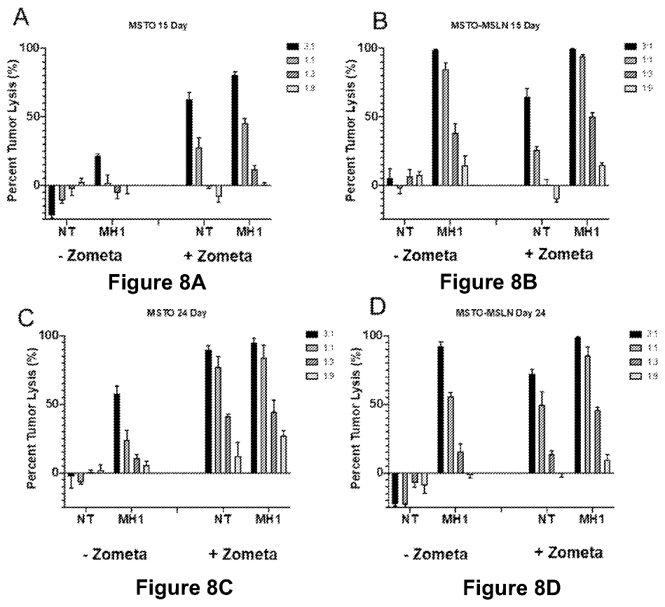

Cytotoxicity was also measured as is described in Example 4 for untransfected and transfected cells. MSTO cells expressing luciferase and no target or MSLN were co-cultured with untransduced cells or cells transduced with a lentiviral vector encoding MSLN TFP at the ratio shown with or without zometa after 15 and 24 days of co-culture. The results are shown in FIG. 8. The function of both MSLN TFP and Vδ2 TCR are demonstrated by the cytotoxicity against MSLNhi cells (MSTO-MSLN-Luc) in the absence of Zometa, and the cytotoxicity against MSLNlow cells (MSTO) in presence of Zometa, respectively.

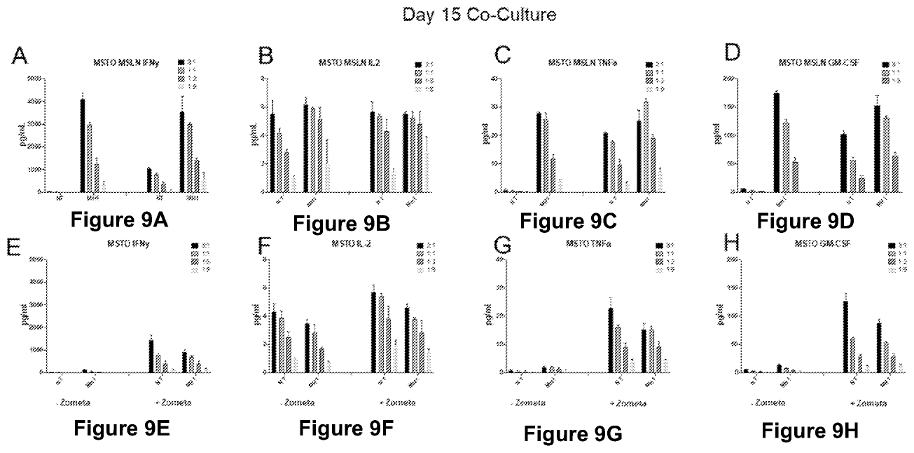

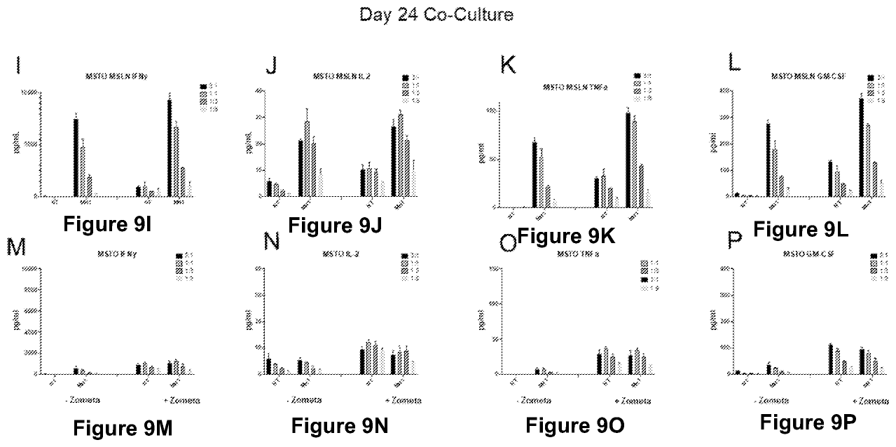

The induction of cytokine production by target cells was also measured as is described in Example 3. MSTO target cells expressing no target or MSLN were co-cultured with untransduced cells or cells transduced with a lentiviral vector encoding MSLN TFP at the ratios shown. The cells were cultured in the presence or absence of zometa. Levels of IL-2, IFN-γ, GM-CSF, and TNF-α were measured. Consistent with cytotoxicity results in FIG. 8, function of both MSLN TFP and Vδ2 TCR are demonstrated by the cytokine production against MSLNhi cells (MSTO-MSLN-Luc) in the absence of Zometa, and the cytokine production against MSLNlow cells (MSTO) in presence of Zometa, respectively. The level of cytokine production from the same cells (MSLN TFP for instance) is always lower in Vδ2 TCR-activation (MSTO cells co-culture, FIGS. 9E and 9M) than in MSLN TFP activation (MSTO-MSLN cells co-culture) (FIGS. 9A and 9I).

### Example 7: Allogeneic TFP Expressing Cells Prepared According to Approach 3

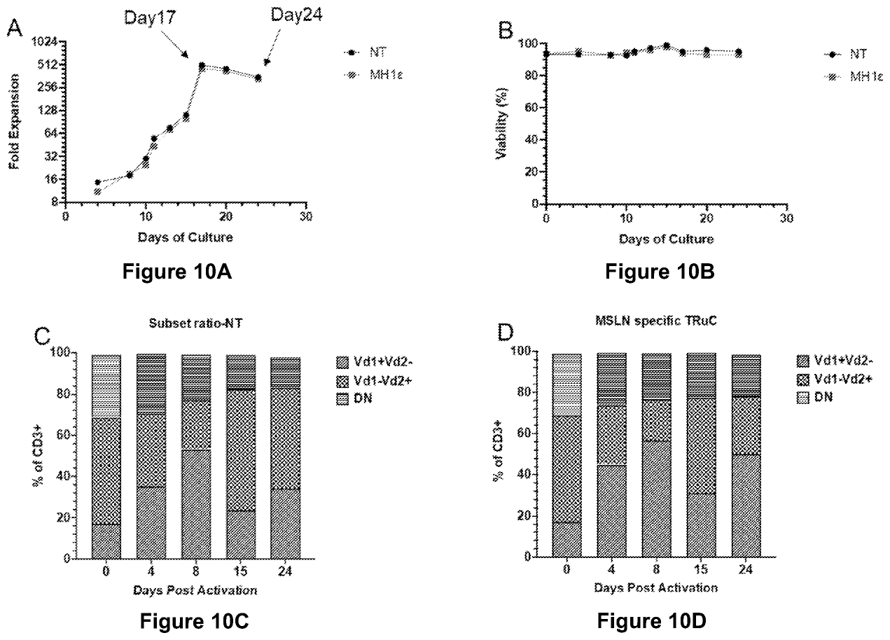

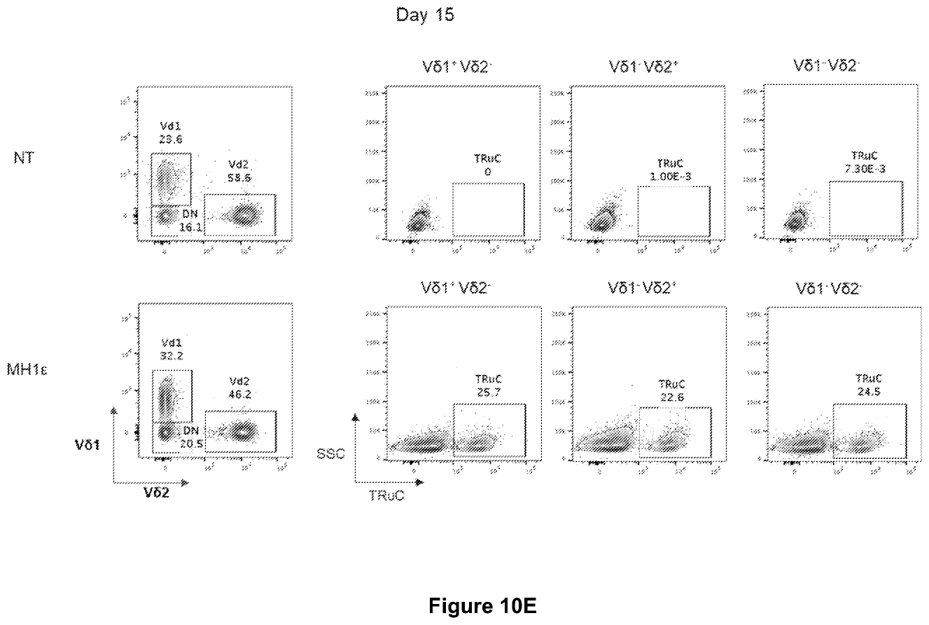

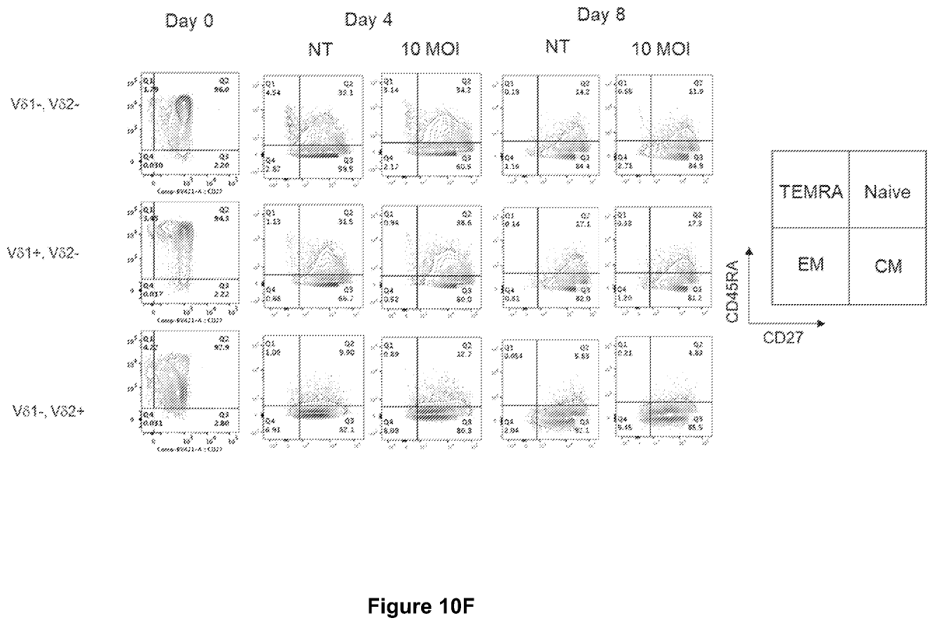

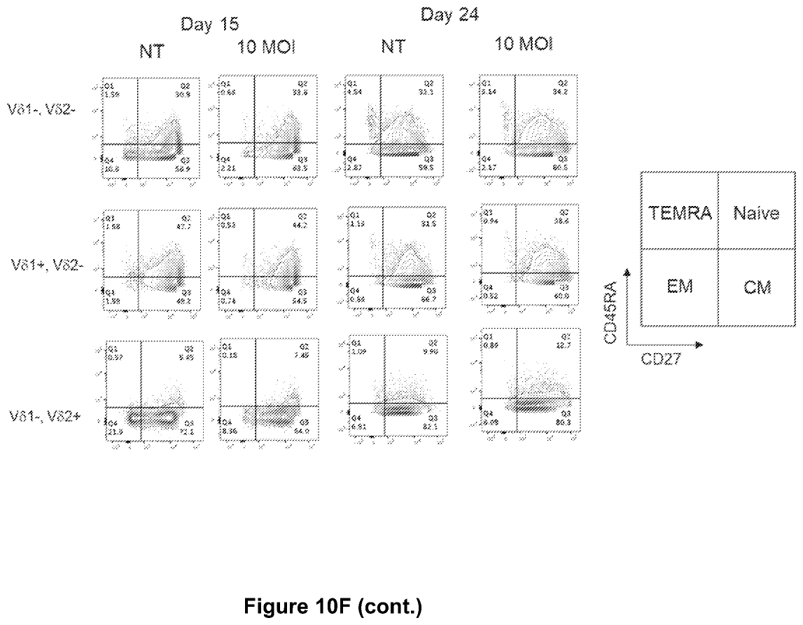

γδ T cells from one donor were transfected with a MSLN TFP according to the methods described in Example 1, Approach 3. As is described in Example 1, γδ T cells were isolated from PBMCs by negative selection. The cells were activated with IL-7 and IL-15 in the presence of anti-CD3 and anti-CD28-attached magnetic beads (TransAct beads), transduced after 1 day with a lentiviral vector encoding a MSLN TFP described above. The cells were then expanded in the presence of IL-7 and IL-15, and harvested on days 15 and 24. FIGS. 10A and 10B show the expansion and viability of the cells post transduction, respectively. FIGS. 10C and 10D show the proportion of Vδ1−Vδ2−, Vδ1+Vδ2−, and Vδ1−Vδ2+ cells at 4, 8, 15 and 24 days post-activation for non-transduced and transduced cells. FIG. 10E shows the proportion of the Vδ1−Vδ2−, Vδ1+Vδ2−, and Vδ1−Vδ2+ subsets that are positive for the TFP at day 15 post-activation by flow cytometry. FIG. 10F shows the differentiation state (whether the cell is naïve, central memory, effector memory, or TEMRA) as measured by anti-CD45RA and anti-CD27 flow cytometry for the Vδ1−Vδ2−, Vδ1+Vδ2−, and Vδ1−Vδ2+ subsets at 0, 4, 8, 15 and 24 days post-activation. Approach 3 demonstrates robust expansion of purified gd T cells (500-fold) (FIG. 10A). Although expansion slows down after day 17, viability of cells remains high in culture (FIG. 10B). Similar to Approach 2, Approach 3 also expands all subsets with similar profiles for NT and MSLN TFP transduced cells (FIGS. 10C and 10D). Transduction of Vδ1−Vδ2−, Vδ1+Vδ2−, and Vδ1−Vδ2+ subsets are detected at similar level (FIG. 10E). All subsets differentiate from naïve stage on day 0 towards CM stage at day 24 (FIG. 10F).

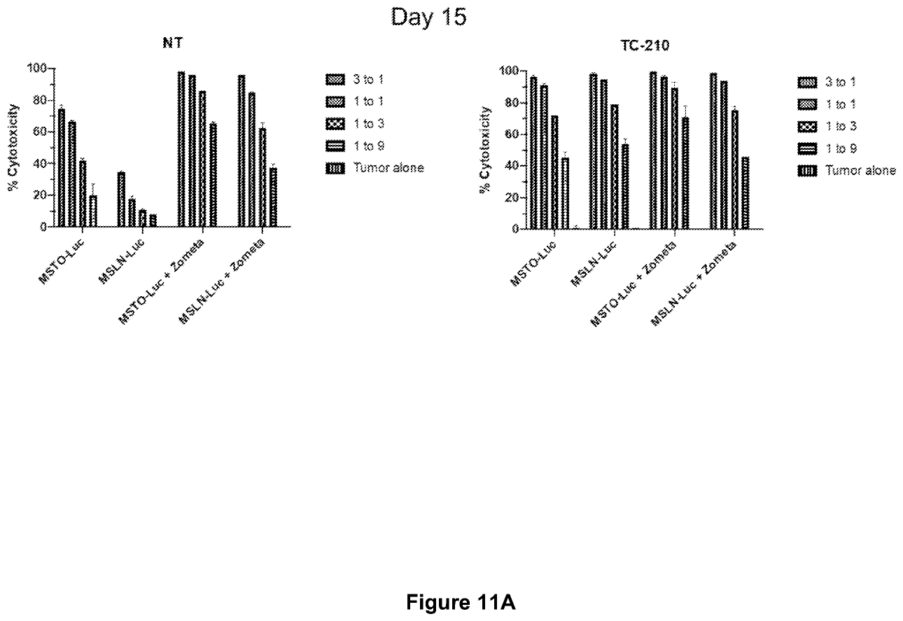

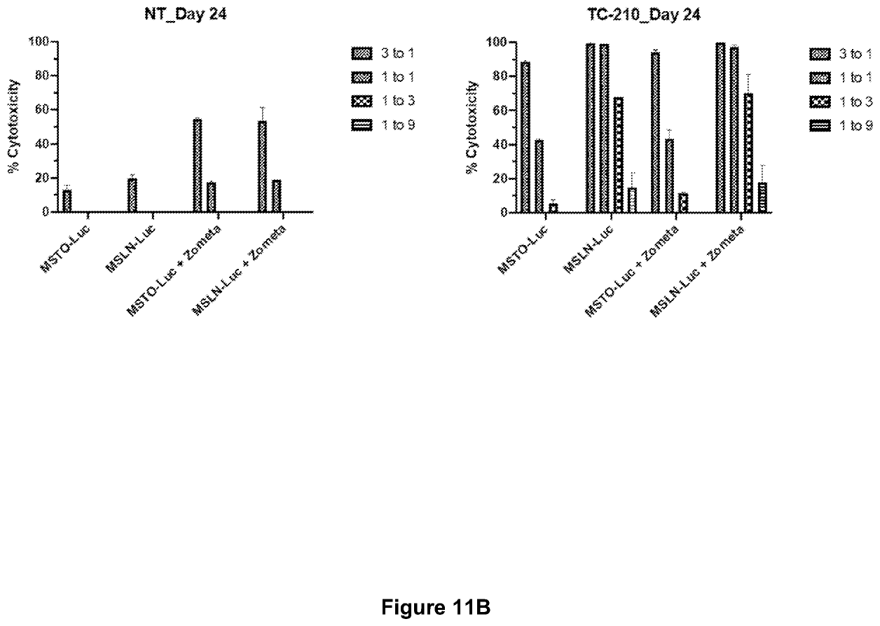

Cytotoxicity was also measured as is described in Example 4 for untransfected and transfected cells. MSTO cells expressing luciferase and no target or MSLN were co-cultured with untransduced cells or cells transduced with a lentiviral vector encoding MSLN TFP at the ratio shown with or without zometa after 15 and 24 days of co-culture. The results are shown in FIG. 11. The function of both MSLN TFP and Vδ2 TCR are demonstrated by the cytotoxicity against MSLNhi cells (MSTO-MSLN-Luc) in the absence of Zometa, and the cytotoxicity against MSLNlow cells (MSTO) in presence of Zometa, respectively. Noticeably, day 15 cells show higher level of baseline cytotoxicity than day 24 cells.

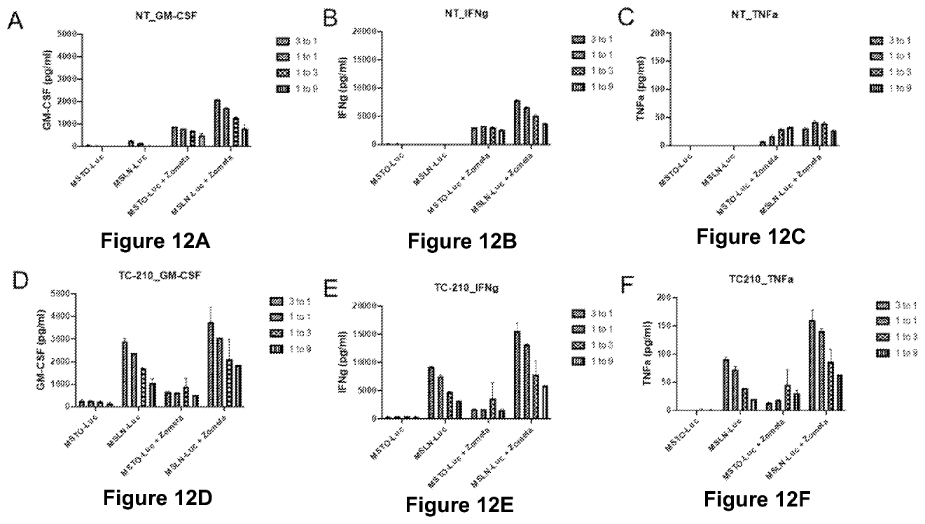

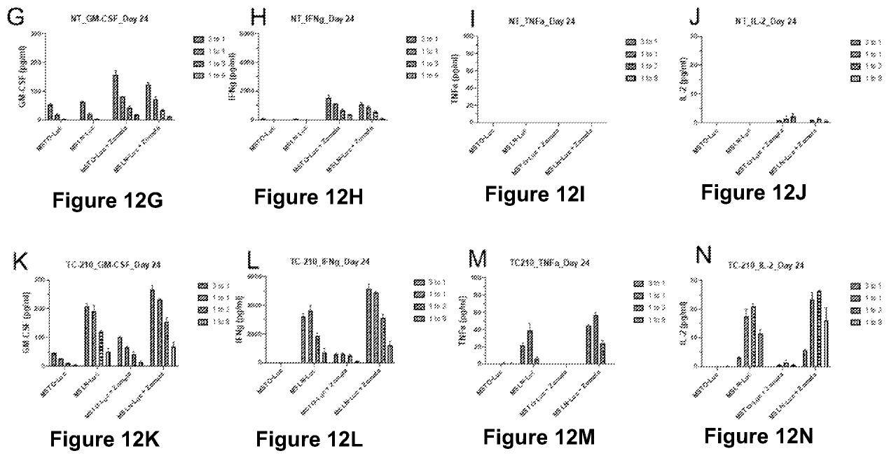

The induction of cytokine production by target cells was also measured as is described in Example 3. MSTO target cells expressing luciferase and no target or MSLN were co-cultured with untransduced cells or cells transduced with a lentiviral vector encoding MSLN TFP at the ratios shown. The cells were cultured in the presence or absence of zometa. Levels of IL-2, IFN-γ, GM-CSF, and TNF-α were measured. Consistent with cytotoxicity results in FIG. 11, function of both MSLN TFP and Vδ2 TCR are demonstrated by the cytokine production against MSLNhi cells (MSTO-MSLN-Luc) in the absence of Zometa, and the cytokine production against MSLNlow cells (MSTO) in presence of Zometa, respectively. The level of cytokine production from the same cells (MSLN TFP for instance) is always lower in Vδ2 TCR-activation (MSTO cells co-culture) than in MSLN TFP activation (MSTO-MSLN cells co-culture) (FIGS. 12D and 12K).

### Example 8: Assaying Different Production Conditions for Allogeneic TFP Expressing γδ T Cells

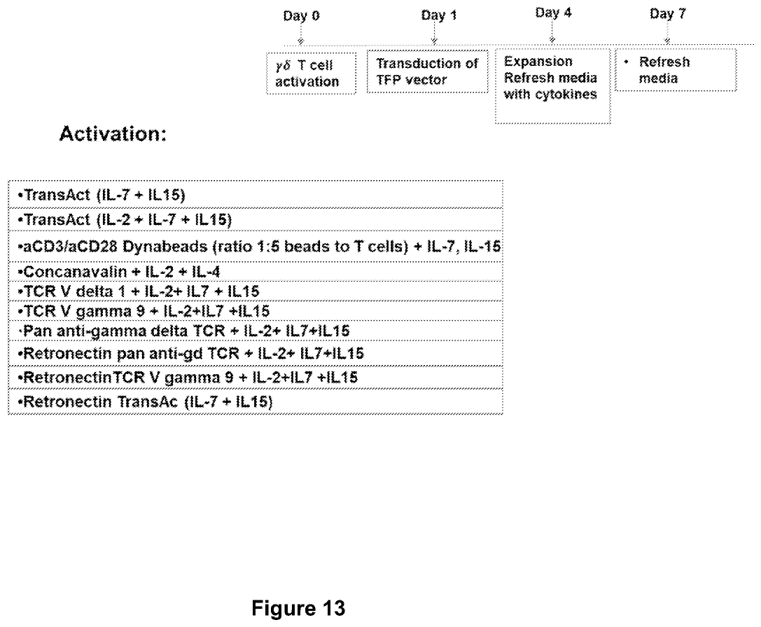

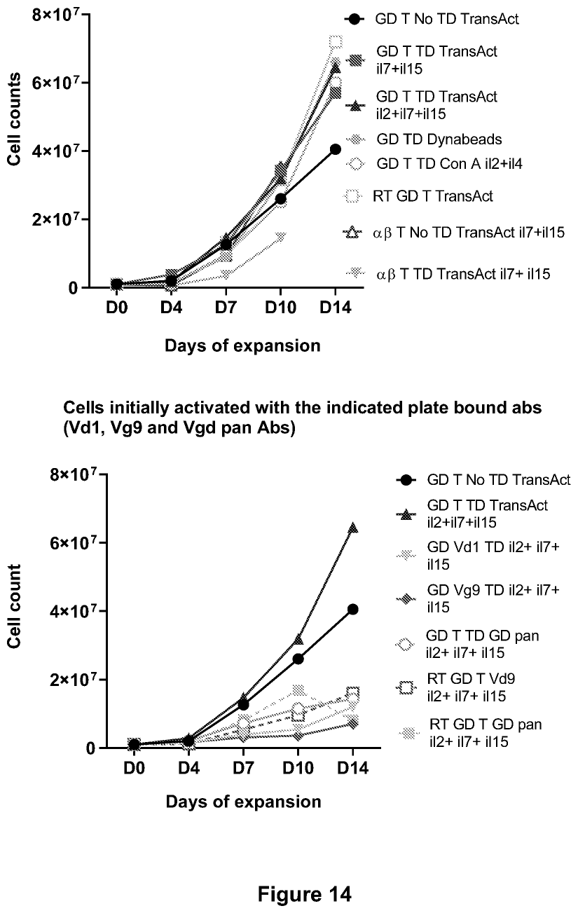

γδ T cells obtained from Hemacare were activated and expanded according to the schematic shown in FIG. 13. Cells were activated in one of 10 activation conditions shown below in Table 3, were transduced with a lentivirus expressing an anti-mesothelin TFP (also referred to as MH1e TFP or TC-210) after 24 hours as described in Example 1, and expanded in fresh media containing the cytokines but not other factors from the activating conditions at day 4. Media was refreshed at day 7. For conditions 5-10, cells were contacted with plate-bound anti-TCR V delta 1, anti-TCR V gamma 9, pan anti-gamma delta TCR, and or retronectin in a 96-well plate until day 4.

TABLE 3
Activation conditions for γδ T cells

| | |
|---|---|
| 1 | TransAct (anti-CD3/anti-CD28) (IL-7 + IL15) |
| 2 | TransAct (IL-2 + IL-7 + IL15) |
| 3 | anti-CD3/anti-CD28 Dynabeads (ratio 1:5 beads |
| | to T cells) + IL-7, IL-15 |
| 4 | Concanavalin A + IL-2 + IL-4 |
| 5 | anti-TCR V delta 1 + IL-2 + IL7 + IL15 |
| 6 | anti-TCR V gamma 9 + IL-2 + IL7 + IL15 |
| 7 | Pan anti-gamma delta TCR + IL-2 + IL7 + IL15 |
| 8 | Retronectin + pan anti-gamma delta TCR + IL-2 + IL7 + IL15 |
| 9 | Retronectin + anti-TCR V gamma 9 + IL-2 + IL7 + IL15 |
| 10 | Retronectin + TransAct (IL-7 + IL15) |

IL-7 12.5 ng/ml
IL-15 12.5 ng/ml
IL-2 100 units/ml
IL-4 5 ng/ml
Concavalin A 1 μg/ml

αβ T cells were prepared as previously described. Expansion through day 14 is shown in FIG. 14. As is shown, transduced γδ TFP T cells activated with transact, dynabeads, or Concanavalin A showed greater expansion than cells activated with plate bound antibodies or retronectin.

### Phenotyping and Verification of TFP Expression by Cell Staining

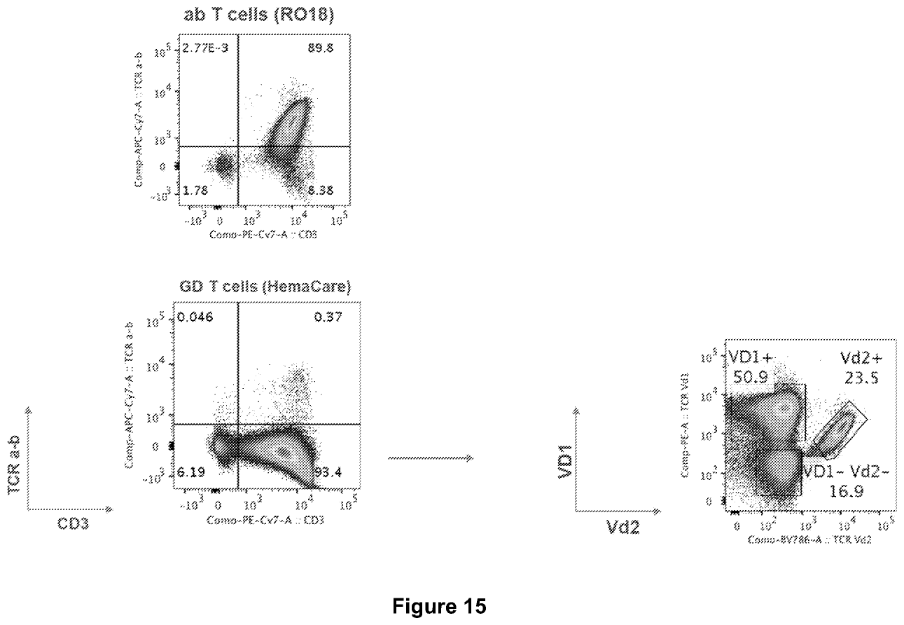

The phenotype of αβ and γδ T cells used in this example was examined by flow cytometry. Cell surface expression of CD3 was visualized with anti-human CD3 (clone UCHT1), cell surface expression of TCR Vδ1 was measured with anti-human TCR Vδ1 (clone REA173), cell surface expression of TCR Vδ2 was measured with anti-human TCR Vδ2 (clone B6), and cell surface expression of the αβ TCR was visualized with anti-human ββ TCR (clone IP26). FIG. 15 shows cells stained prior to activation. Both αβ and γδ T cells are positive for CD3 staining whereas only αβ T cells are positive for the αβ TCR. About half of the γδ T cells are VD1+ and about a quarter are VD2+.

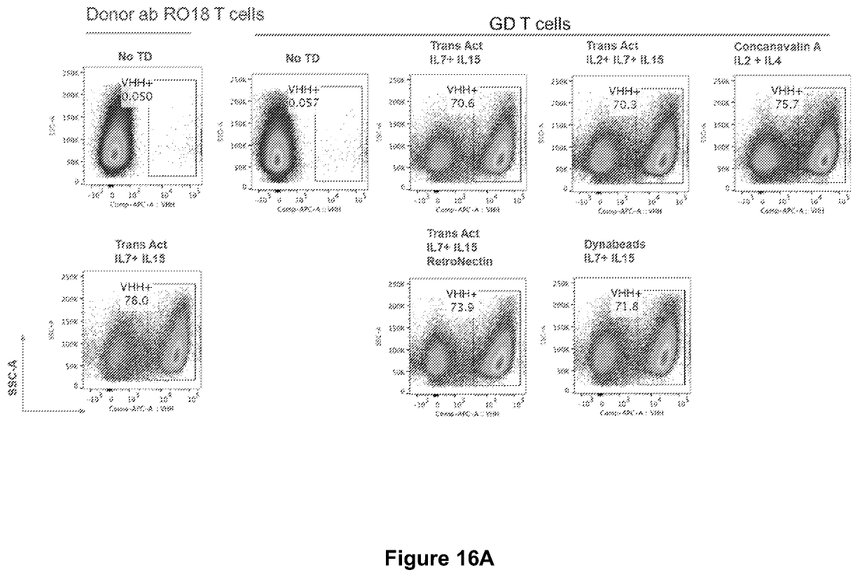

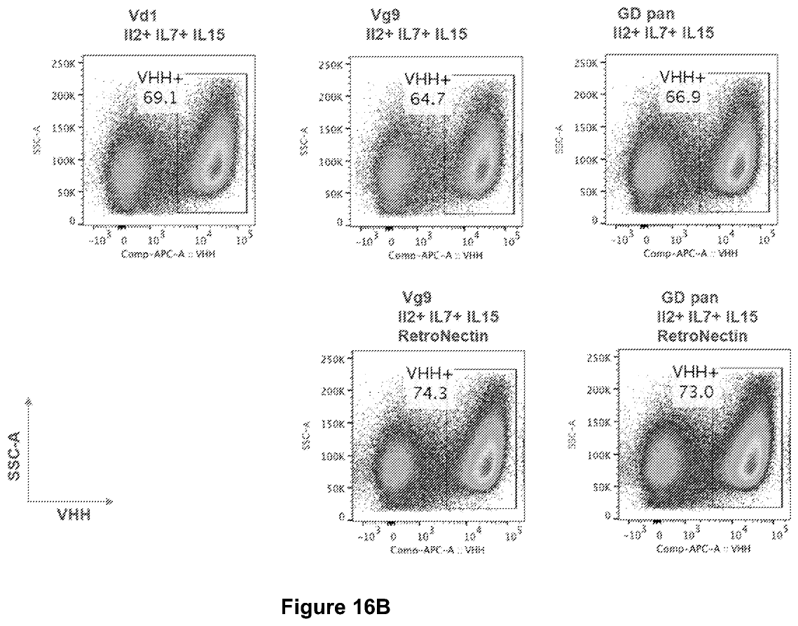

Following lentiviral transduction, expression of TFPs was confirmed by flow cytometry on day 8 post activation, with MonoRab™ Rabbit Anti-Camelid VHH Antibody [iFluor647] (GenScript, clone 96A3F5) for MSLN-specific TFP. As is shown in FIG. 16, high transduction efficiency was seen in all activation conditions.

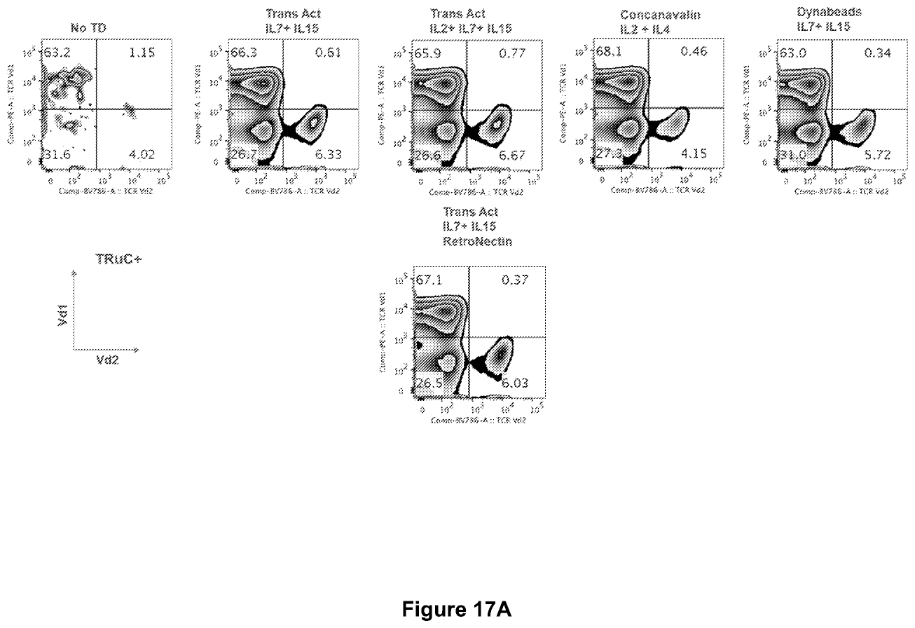

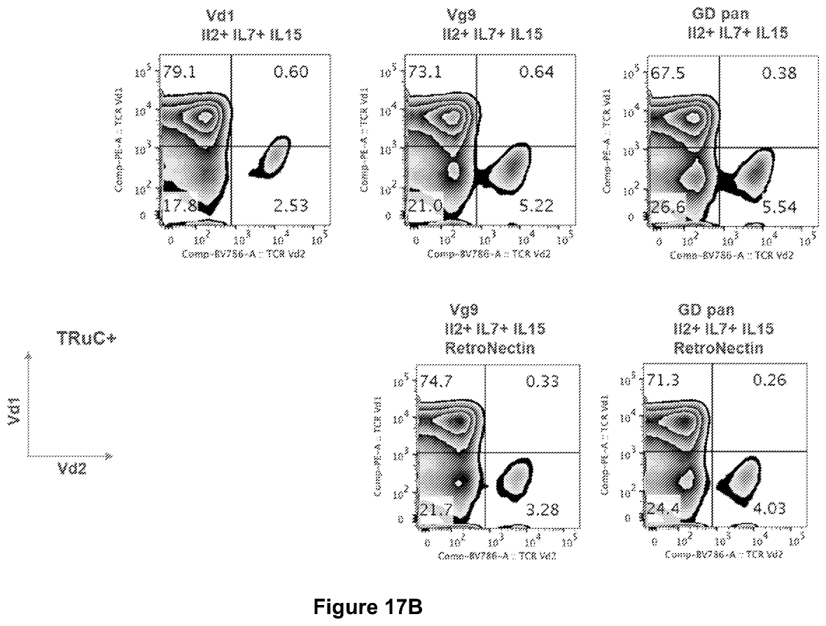

The phenotype of γδ T cells was also evaluated after transduction using the same techniques described above (to measure cell surface staining of Vδ1 and Vδ2). In all conditions, a high level of Vδ1+ cells were seen (i.e., at least 63%) as is shown in FIG. 17.

### Luciferase-Based Cytotoxicity Assay

Human tumor cell line C30 based target cells, expressing no target or MSLN were generated by transduction with lentivirus encoding the human MSLN. Target cells stably expressing desired target antigens were selected by application of antibiotics matched to the resistance gene encoded by the lentivirus. The target cells were further modified to overexpress firefly luciferase via transduction with firefly luciferase encoding lentivirus followed with antibiotic selection to generate stable cell line.

The target cells were plated at 10000 cells per well in 96-well plate. The TFP T or control cells, taken at day 11, were added to the target cells at different effector-to-target ratios. The mixture of cells was then cultured for 24 hours at 37° C. with 5% CO₂ before the luciferase enzymatic activity in the live target cells is measured by the Bright-Glo® Luciferase Assay System (Promega®, Catalogue number E2610). The cells are spun into a pellet and resuspended in medium containing the luciferase substrate. The percentage of tumor cell killing is then calculated with the following formula: % Cytotoxicity=100%×[1−RLU (Tumor cells+T cells)/RLU (Tumor cells)].

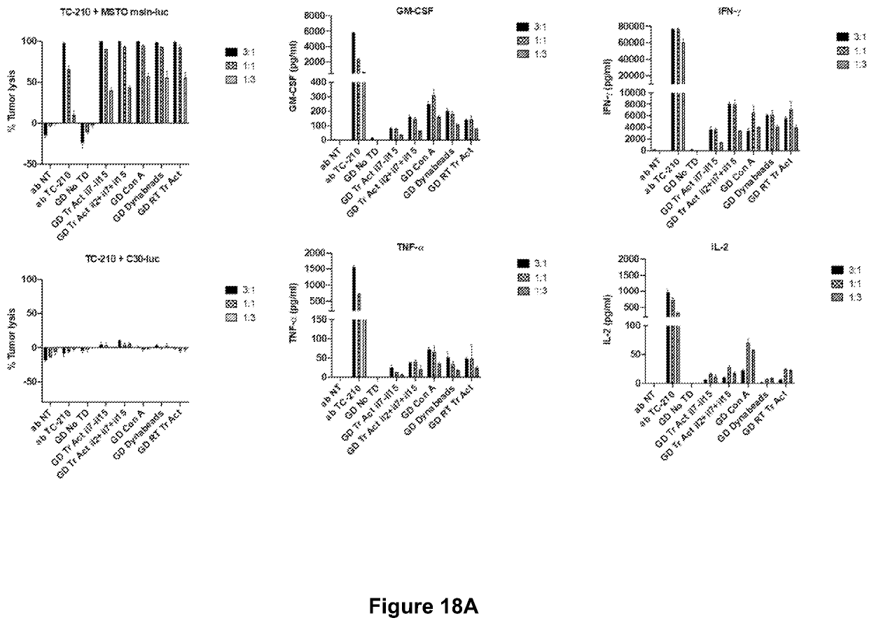

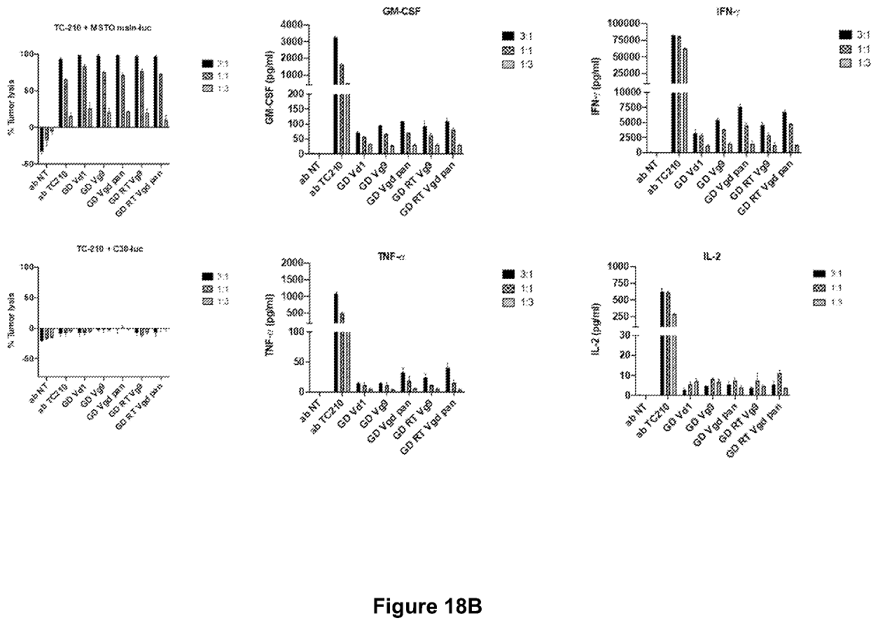

FIGS. 18A and 18B show that cytotoxic capacity of γδ TFP T cells generated in each of the 10 different conditions was at least comparable to that of TFP T cells, while FIG. 18A shows that, for γδ TFP T cells generated with transact, dynabeads, or Concanavalin A, cytotoxicity for γδ TFP T cells was higher than for TFP T cells, especially at 1:1 and 1:3 ratios.

### Cytokine Secretion Measurement by MSD

A measure of effector T-cell activation and proliferation associated with the recognition of cells bearing cognate antigen is the production of effector cytokines such as interleukin-2 (IL-2) and interferon-gamma (IFN-γ), granulocyte-macrophage colony-stimulating factor (GM-CSF) and tumor necrosis factor alpha (TNF-α). However, excessive production of cytokines may contribute to cytokine release syndrome and indicate a less favorable safety profile.

Target-specific cytokine production including IL-2, IFN-γ, GM-CSF, and TNF-α by monospecific TFP T cells activated and expanded for 11 days in each of the 10 different conditions was measured from supernatants harvested 48 hours after the co-culture of T cells with various tumor cells using the U-PLEX® Biomarker Group I (hu) Assays (Meso Scale Diagnostics®, LLC, catalog number: K15067L-4). FIGS. 18A and 18B show that, in all conditions, cytokine production by γ6 TFP T cells was reduced compared to that of TFP expressing αβ TFP T cells, suggesting that γδ TFP T cells generated according to the methods described herein may have a better safety profile than αβ TFP T cells.

### Example 9: γδ TFP T Cells with Different Co-Factors

Constructs Used in this Study

In some embodiments, TFP constructs (e.g., MH1e-TFP expressing constructs described above) are contained in a vector that further contains a sequence encoding a second peptide selected from (i) a PD-1-CD28 fusion comprising the extracellular and transmembrane domain of PD-1 fused to the intracellular domain of CD28 (e.g., SEQ ID NO: 86); (ii) a PD-1-CD28-IL15-Rα fusion comprising the extracellular and transmembrane domain of PD-1 fused to the intracellular domain of CD28 fused to the intracellular domain of IL15-Rα at the C-terminus of CD28 (e.g., SEQ ID NO: 85); (iii) an IL-15-IL15-Rα fusion (e.g., SEQ ID NO: 84); or soluble IL-15 (e.g., SEQ ID NO: 81). The second peptide may be encoded in the same open reading frame and separated by a self-cleaving peptide (e.g., a P2A or a T2A self-cleaving peptide). In some embodiments, the vector further comprises a third peptide. In some embodiments, the second peptide is a PD-1-CD28-IL15-Rα fusion and the third peptide is soluble IL-15. The third peptide may be encoded in the same open reading frame as the TFP and second peptide and separated from the second peptide by a self-cleaving peptide (e.g., a P2A or a T2A self-cleaving peptide). The constructs used in this Example are shown in Table 4 below.

TABLE 4
Constructs used in Example 9

| Construct | ID# |
|---|---|
| MH1-CD3 epsilon (TC-210; MH1ε TFP) | TC-210 |
| MH1ε TFP T2AW PD1(EC-TM)CD28(IC) | 70020 |
| MH1ε T2AW PD1(EC-TM)CD28(IC)IL15Ra(IC) | 10299 |
| MH1ε T2A PD1(EC-TM)CD28(IC)IL15Ra(IC) P2A IL15 | 70080 |
| MH1ε T2AW IL15 | 70050 |
| MH1ε T2AW IL15-IL15Ra | 70079 |

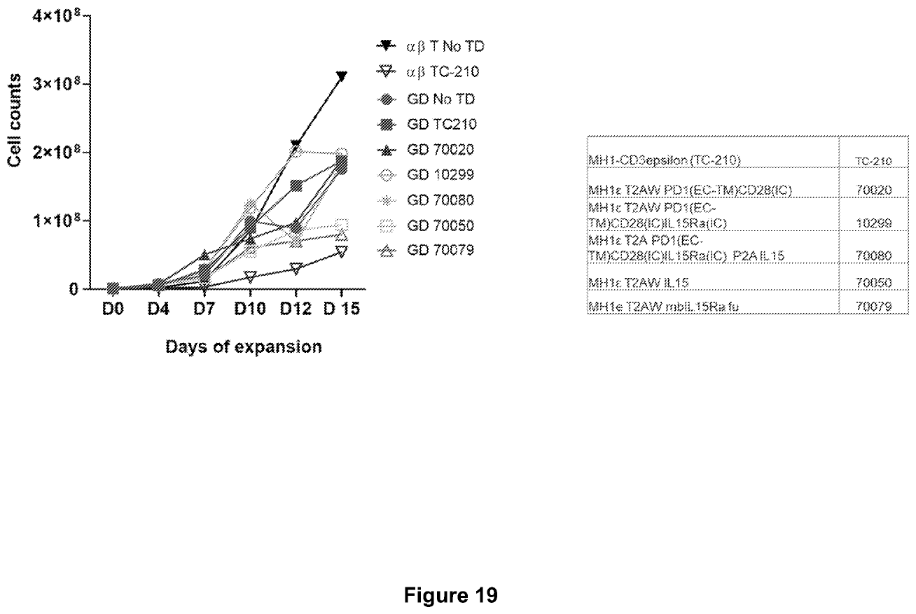

γδ T cells obtained from Hemacare were activated and expanded according to condition 1 in Example 8—cells were activated in media with transact, 12.5 ng/ml IL-7, and 12.5 ng/ml IL-15. Cells were transfected with the vectors described above on day 1, and on day 4, cells were switched to media having IL-7 and IL-15 but not having transact for expansion. Media was then refreshed on day 7. αβ T cells were prepared as previously described. Expansion of the T cells is shown in FIG. 19. Expansion of all of the transduced γδ T cells was superior to that of T cells transduced with TC-210.

### Phenotyping and Verification of TFP Expression by Cell Staining

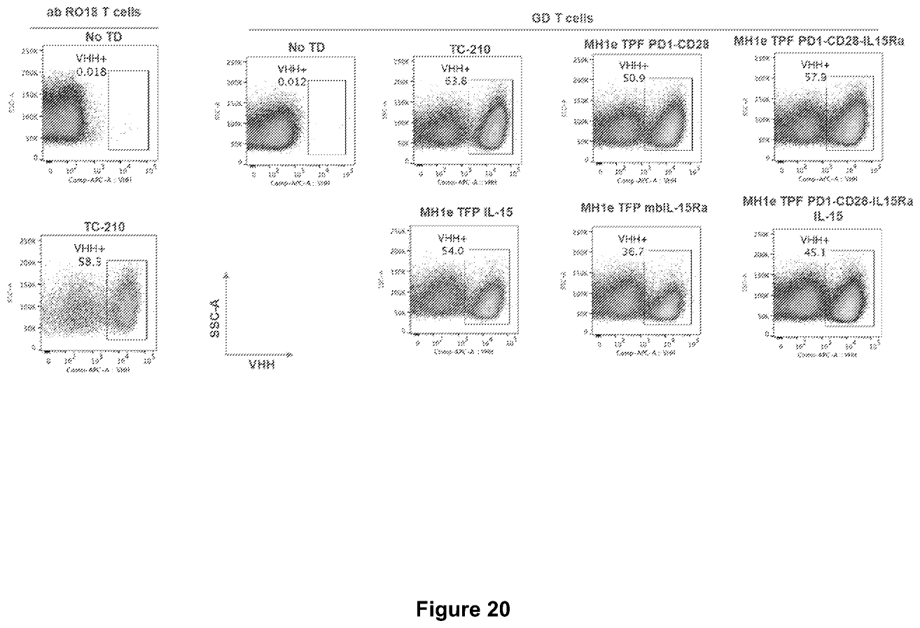

Following lentiviral transduction, expression of TFPs was confirmed by flow cytometry on day 8 post-activation, with MonoRab™ Rabbit Anti-Camelid VHH Antibody [iFluor647] (GenScript, clone 96A3F5) for MSLN-specific TFP. As is shown in FIG. 20, high transduction efficiency was seen with all constructs, and transduction efficiency in γδ T cells with all vectors was comparable to αβ T cells transduced with a vector encoding TC-210.

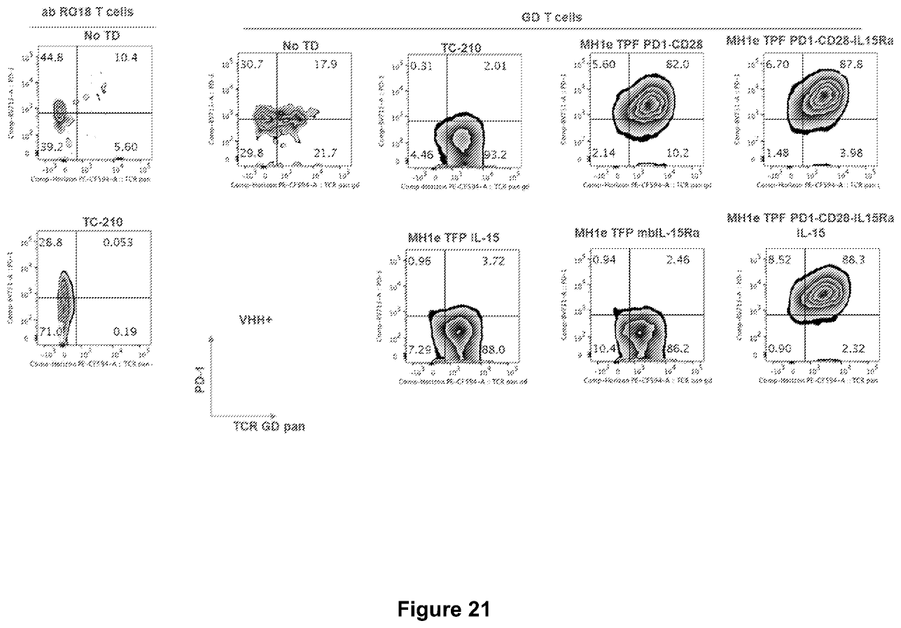

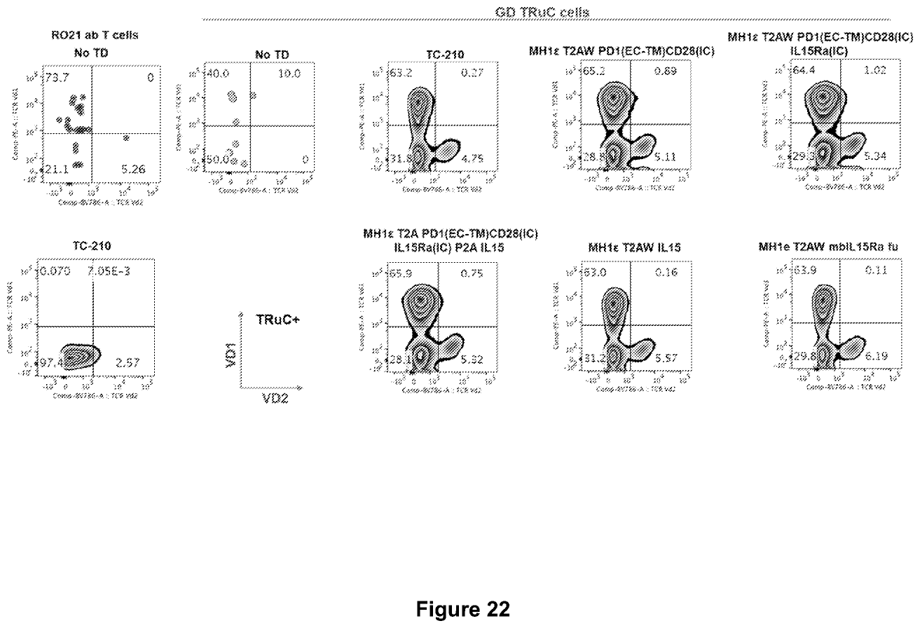

The phenotype of γδ T cells was also evaluated after transduction with an anti-PD-1 antibody, to measure the proportion of PD-1 positive cells (e.g., for those cells transduced with PD-1 containing constructs), and with a pan γδ TCR antibody, to assess cell surface expression of the γδ TCR. As is shown in FIG. 21, a high proportion of γδ TCR+ cells was seen in TFP-transduced γδ T cells and a high proportion of PD-1+ cells was seen in γδ T cells transduced with a TFP vector containing a PD-1 (i.e., transduced with vectors encoding PD-1-CD28 or PD-1-CD-28-IL15-Ra fusions).

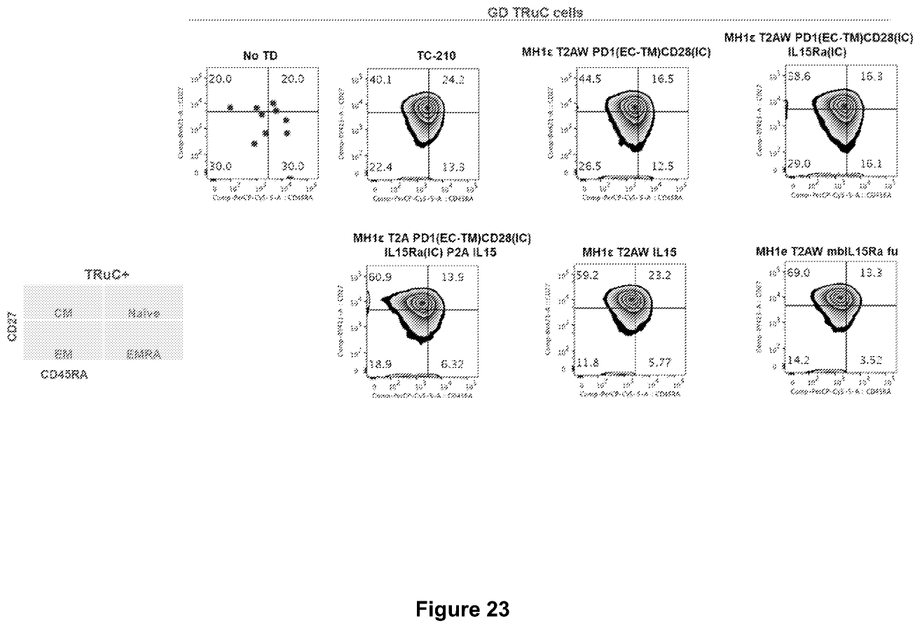

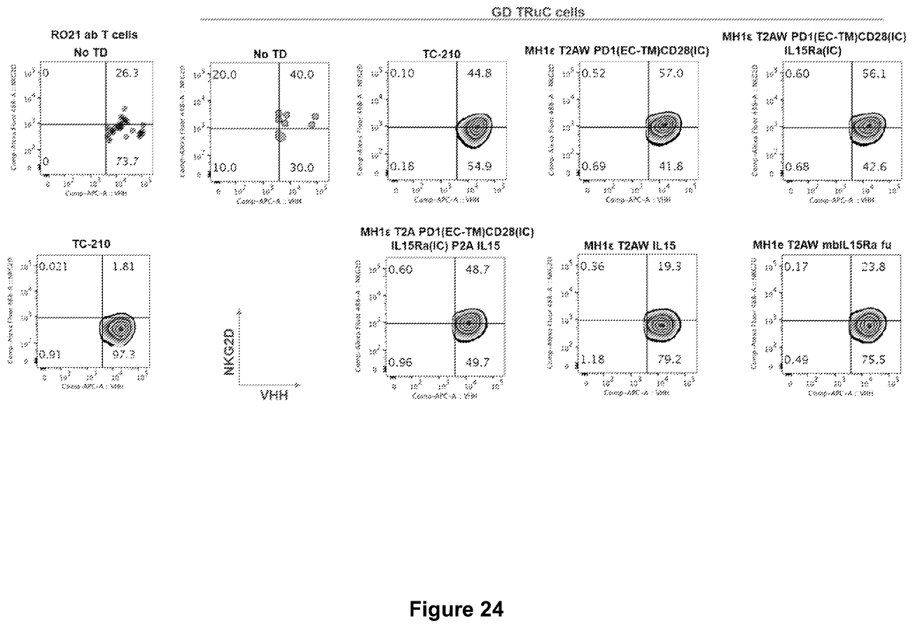

The memory status of the T cells was also determined by flow cytometry to detect cell surface levels of CD45RA and CCR7 as is shown in FIG. 23. γδ T cells expressing the MH1e TFP with secreted IL-15, the IL-15-IL15-Rα fusion or the PD-1-CD28-IL15-Rα fusion with secreted IL-15 showed an increased central memory cell population relative to cells expressing MH1e TFP, the MH1e TFP with the PD-1-CD28 fusion, or the MH1e TFP with the PD-1-CD28-IL15-Rα fusion without secreted IL-15.

The proportion of NKG2D positive cells was also determined by flow cytometry. As is shown in FIG. 24, γδ T cells expressing any of the TFP constructs had a dramatically increased population of NKG2D positive cells relative to T cells transduced with TC-210

### Luciferase-Based Cytotoxicity Assay

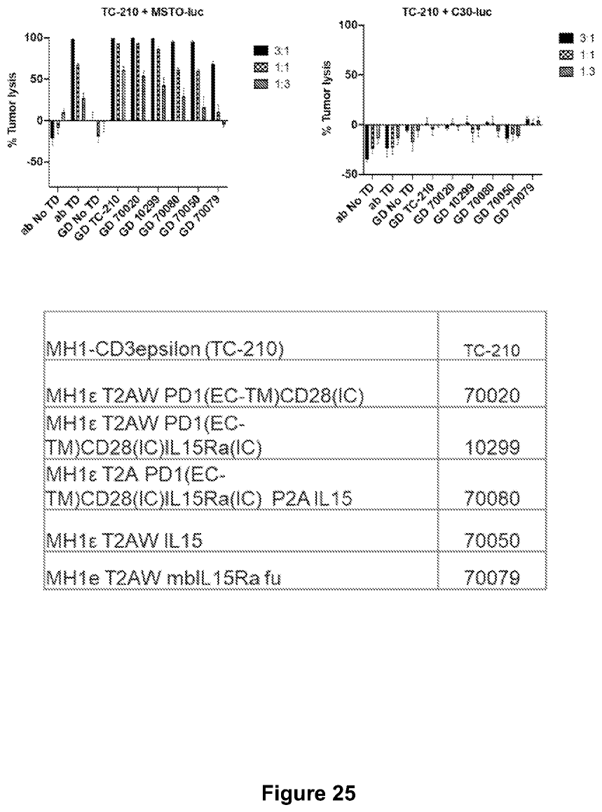

A luciferase-based cytotoxicity assay was performed as described in Example 8. FIG. 25 show that the cytotoxic capacity of γδ TFP T cells expressing TC-210, the MH1e TFP with the PD-1-CD28 fusion protein, or the MH1e TFP with the PD-1-CD-28-IL-15Rα fusion exhibit greater cytotoxicity than αβT cells expressing TC-210, especially at 1:1 and 1:3 ratios.

### Cytokine Secretion Measurement by MSD

A cytokine secretion assay was performed as described in Example 8. Levels of IL-2, IFN-γ, GM-CSF, TNF-α, CCL4, IL-15, CCL3, and IL-17A were measured.

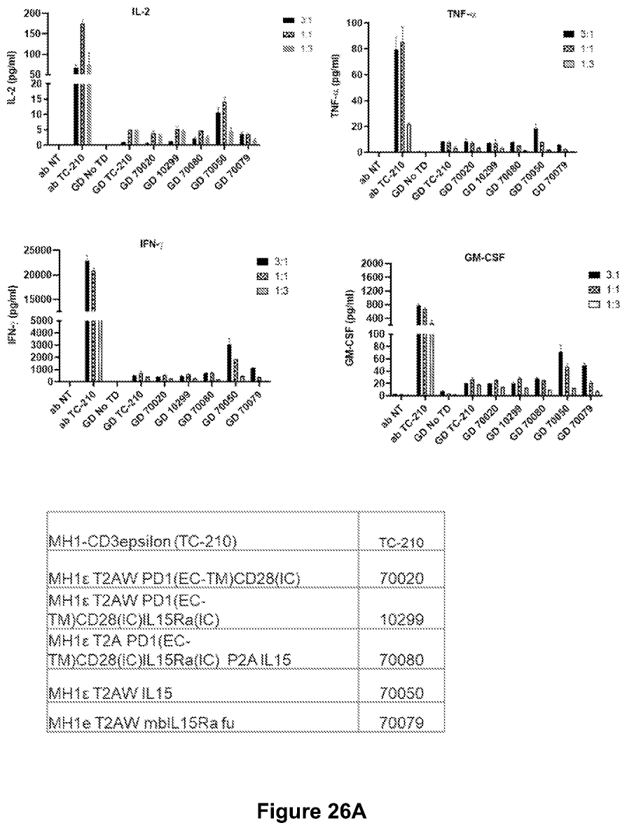

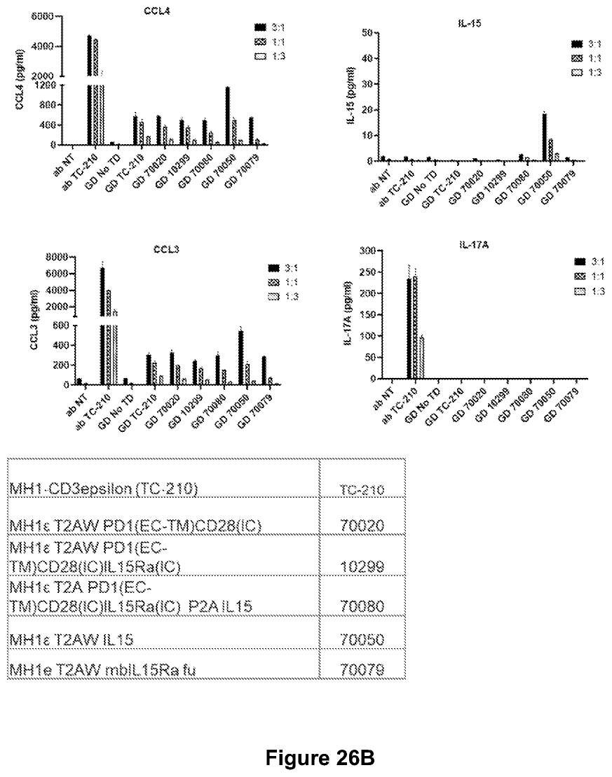

FIGS. 26A and 26B show that, in all conditions, cytokine production by γδ TFP T cells was reduced compared to that of TFP expressing αβ TFP T cells, suggesting that γδ TFP T cells generated according to the methods described herein may have a better safety profile than αβ TFP T cells. Greater cytokine production was seen with the IL-15 secreting construct than the other constructs, and all constructs showed secretion of IL-2, demonstrating the ability to persist in vivo.

### OTHER EMBODIMENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

What is claimed is:

1. A cell comprising a recombinant nucleic acid encoding a T cell receptor (TCR) fusion protein (TFP) comprising:
(a) a TCR-integrating subunit comprising:
at least a portion of a TCR extracellular domain,
(ii) a transmembrane domain, and
(iii) an intracellular domain comprising a stimulatory domain from an intracellular signaling domain of CD3 epsilon, CD3 gamma, or CD3 delta, or CD3 zeta; and
(b) an antibody or fragment thereof comprising an antigen binding domain;
wherein the TCR-integrating subunit and the antibody are operatively linked, wherein the cell is a gamma delta (γδ) T cell, and wherein the expressed TFP functionally incorporates into a γδ TCR complex of the γδ T cell.

2. The cell of claim 1, wherein the antibody or fragment thereof is human or humanized.

3. The cell of claim 1 or 2, wherein the antibody or fragment thereof is murine.

4. The cell of any one of claims 1-3, wherein the antibody or fragment thereof binds to a cell surface antigen.

5. The cell of any one of claims 1-4, wherein the antibody or fragment thereof binds to a cell surface antigen on the surface of a tumor cell.

6. The cell of any one of claims 1-5, wherein the antibody is a full length antibody.

7. The cell of any one of claims 1-5, wherein the antibody fragment is a scFv, a single domain antibody domain, a V_{H} domain or a V_{L} domain.

8. The cell of any one of claims 1-7, wherein an antigen binding domain is selected from a group consisting of an anti-CD19 binding domain, anti-B-cell maturation antigen (BCMA) binding domain, anti-mesothelin (MSLN) binding domain, an anti-MUC16 binding domain, an anti-HER2 binding domain, an anti-PSMA binding domain, an anti-CD70 binding domain, an anti-CD79B binding domain, an anti-PD-L1 binding domain, an anti-Nectin-4 binding domain, an anti-Trop-2 binding domain, an anti-GPC3 binding domain, and an anti-BAFF receptor binding domain.

9. A cell comprising a recombinant nucleic acid encoding T cell receptor (TCR) fusion protein (TFP) comprising:
(a) a TCR-integrating subunit comprising:
at least a portion of a TCR extracellular domain,
(ii) a transmembrane domain, and
(iii) an intracellular domain comprising a stimulatory domain from an intracellular signaling domain of CD3 epsilon, CD3 gamma, CD3 delta, or CD3 zeta; and
(b) an antigen binding domain comprising a ligand or a fragment thereof;
wherein the TCR-integrating subunit and the antigen binding domain are operatively linked, wherein the cell is a γδ T cell; and
wherein the expressed TFP functionally incorporates into a γδ TCR complex of the γδ T cell.

10. The cell of claim 9, wherein the ligand or fragment thereof is capable of binding to an antibody or fragment thereof.

11. The cell of claim 10, wherein the ligand is capable of binding an Fc domain of the antibody.

12. The cell of claim 10, wherein the ligand is capable of selectively binding an IgG1 antibody.

13. The cell of claim 10, wherein the binding is capable of specifically binding an IgG4 antibody.

14. The cell of any one of claims 9-13, further comprising a nucleic acid sequence encoding an antibody or fragment thereof capable of being bound by the binding ligand.

15. The cell of claim 9, wherein the ligand or fragment thereof binds to a receptor or polypeptide expressed on the surface of a cell.

16. The cell of claim 15, wherein the ligand binds to the receptor of a cell.

17. The cell of claim 15, wherein the ligand binds to the polypeptide expressed on a surface of a cell.

18. The cell of any one of claims 15-17, wherein the ligand or fragment thereof is a Natural Killer Group 2D (NKG2D) ligand or a fragment thereof.

19. The cell of any one of claims 15-18, wherein the receptor or polypeptide expressed on a surface of a cell comprises a stress response receptor or polypeptide.

20. The cell of any one of claims 15-19, wherein the receptor or polypeptide expressed on a surface of a cell is an MHC class I-related glycoprotein.

21. The cell of claim 20, wherein the MHC class I-related glycoprotein is selected from the group consisting of MICA, MICB, RAET1E, RAET1G, ULBP1, ULBP2, ULBP3, ULBP4 and combinations thereof.

22. The cell of any one of claims 9-21, wherein the ligand or fragment thereof is a monomer, a dimer, a trimer, a tetramer, a pentamer, a hexamer, a heptamer, an octomer, a nonamer, or a decamer.

23. The cell of claim 22, wherein the ligand or fragment thereof is a monomer or a dimer.

24. The cell of any one of claims 9-22, wherein the antigen binding domain comprises a monomer, a dimer, a trimer, a tetramer, a pentamer, a hexamer, a heptamer, an octomer, a nonamer, or a decamer.

25. The cell of claim 24, wherein the antigen binding domain comprises a monomer or a dimer of the ligand or fragment thereof.

26. The cell of any one of claims 9-25, wherein the antigen binding domain does not comprise an antibody or fragment thereof.

27. The cell of any one of claims 9-26, wherein the antigen binding domain does not comprise a variable region.

28. The cell of any one of claims 9-27, wherein the antigen binding domain does not comprise a CDR.

29. The cell of any one of the preceding claims, wherein the TCR-integrating subunit and the antibody domain or the antigen binding domain are operatively linked by a linker sequence.

30. The cell of claim 29, wherein the linker sequence comprises (G₄S)ₙ, wherein n=1 to 4.

31. The cell of any one of the preceding claims, wherein the transmembrane domain is a transmembrane domain from CD3 epsilon, CD3 gamma, CD3 delta, TCR gamma or TCR delta.

32. The cell of any one of the preceding claims, wherein the intracellular domain is derived from at least in part CD3 epsilon, only CD3 gamma, or only CD3 delta.

33. The cell of any one of the preceding claims, wherein the TCR-integrating subunit comprises (i) at least a portion of a TCR extracellular domain, (ii) a TCR transmembrane domain, and (iii) a TCR intracellular domain, wherein at least two of (i), (ii), and (iii) are from the same TCR subunit.

34. The cell of any one of the preceding claims, wherein the TCR extracellular domain comprises an extracellular domain or portion thereof of a protein selected from the group consisting of a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

35. The cell of any one of the preceding claims, wherein the TCR-integrating subunit comprises a transmembrane domain comprising a transmembrane domain of a protein selected from the group consisting of a TCR gamma chain, a TCR delta chain, a CD3 zeta chain, a CD3 epsilon TCR subunit, a CD3 gamma TCR subunit, or a CD3 delta TCR subunit, functional fragments thereof, and amino acid sequences thereof having at least one but not more than 20 modifications.

36. The cell of any one of the preceding claims, wherein the TCR-integrating subunit comprises a TCR intracellular domain comprising a stimulatory domain of a protein selected from an intracellular signaling domain of CD3 epsilon, CD3 gamma or CD3 delta, or an amino acid sequence having at least one modification thereto.

37. The cell of any one of the preceding claims, wherein the TCR-integrating subunit comprises an intracellular domain comprising a stimulatory domain of a functional signaling domain of CD3 zeta, or an amino acid sequence having at least one modification thereto.

38. The cell of any one of the preceding claims, further comprising a sequence encoding a costimulatory domain.

39. The cell of claim 38, wherein the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

40. The cell of any one of claims 1-39, wherein the TCR-integrating subunit comprises an immunoreceptor tyrosine-based activation motif (ITAM) of a TCR-integrating subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto.

41. The cell of claim 40, wherein the ITAM replaces an ITAM of CD3 gamma, CD3 delta, CD3 epsilon, or CD3 zeta.

42. The cell of claim 40 or 41, wherein the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

43. The cell of any one of the preceding claims, wherein the at least one but not more than 20 modifications thereto comprise a modification of an amino acid that mediates cell signaling or a modification of an amino acid that is phosphorylated in response to a ligand binding to the TFP.

44. The cell of any one of the preceding claims, wherein the γδ T cell is an allogenic T cell.

45. The cell of any one of the preceding claims, wherein the γδ T cell is a Vδ1+Vδ2−γ∂T cell.

46. The cell of any one of claims 1-44, wherein the γδ T cell is a Vδ1−Vδ2+γδT cell.

47. The cell of any one of claims 1-44, wherein the γδ T cell is a Vδ1−Vδ2−γδT cell.

48. The cell of any one of claims 1-47, wherein the antigen binding domain is an anti-CD19 binding domain and the TFP comprises the sequence of SEQ ID NO: 22, SEQ ID NO: 24, or SEQ ID NO: 33.

49. The cell of any one of claims 1-47, wherein the antigen binding domain is an anti-MSLN binding domain and the TFP comprises the sequence of SEQ ID NO: 25, SEQ ID NO: 26, SEQ ID NO: 41, or SEQ ID NO: 34.

50. The cell of any one of claims 1-47, wherein the antigen binding domain is an anti-CD70 binding domain and the TFP comprises the sequence of SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, or SEQ ID NO: 80.

51. The cell of any one of claims 1-47, wherein the antigen binding domain comprises a variable domain comprising a light chain complementarity determining region 1 (LC CDR1), a LC CDR2, and a LC CDR3.

52. The cell of claim 51, wherein the LC CDR1 comprises the sequence of SEQ ID NO: 10, the LC CDR2 comprises the sequence of SEQ ID NO: 12, and the LC CDR3 comprises the sequence of SEQ ID NO: 14.

53. The cell of any one of claims 1-47, wherein the antigen binding domain comprises a variable domain comprising a heavy chain complementarity determining region 1 (HC CDR1), a HC CDR2, and a HC CDR3.

54. The cell of claim 53, wherein the HC CDR1 comprises the sequence of SEQ ID NO: 16, the HC CDR2 comprises the sequence of SEQ ID NO: 18, and the HC CDR3 comprises the sequence of SEQ ID NO: 20.

55. The cell of claim 49, wherein the antigen binding domain comprises a variable domain comprising a complementarity determining region (CDR) 1 of SEQ ID NO: 35, CDR2 of SEQ ID NO: 36, and CDR3 of SEQ ID NO: 37.

56. The cell of claim 49, wherein the antigen binding domain comprises a variable domain comprising a CDR1 of SEQ ID NO: 38, CDR2 of SEQ ID NO: 39, and CDR3 of SEQ ID NO: 40.

57. The cell of claim 49, wherein the antigen binding domain comprises a variable domain comprising a CDR1 of SEQ ID NO: 41, CDR2 of SEQ ID NO: 42, and CDR3 of SEQ ID NO: 43.

58. The cell of claim 50, wherein the antigen binding domain comprises a variable domain comprising a CDR1 of SEQ ID NO: 45, CDR2 of SEQ ID NO: 46, and CDR3 of SEQ ID NO: 47.

59. The cell of claim 50, wherein the antigen binding domain comprises a variable domain comprising a CDR1 of SEQ ID NO: 49, CDR2 of SEQ ID NO: 50, and CDR3 of SEQ ID NO: 51.

60. The cell of claim 50, wherein the antigen binding domain comprises a variable domain comprising a CDR1 of SEQ ID NO: 53, CDR2 of SEQ ID NO: 54, and CDR3 of SEQ ID NO: 55.

61. The cell of claim 50, wherein the antigen binding domain comprises a variable domain comprising a CDR1 of SEQ ID NO: 57, CDR2 of SEQ ID NO: 58, and CDR3 of SEQ ID NO: 59.

62. The cell of claim 50, wherein the antigen binding domain comprises a variable domain comprising a CDR1 of SEQ ID NO: 61, CDR2 of SEQ ID NO: 62, and CDR3 of SEQ ID NO: 63.

63. The cell of claim 50, wherein the antigen binding domain comprises a variable domain comprising a CDR1 of SEQ ID NO: 65, CDR2 of SEQ ID NO: 66, and CDR3 of SEQ ID NO: 67.

64. The cell of claim 50, wherein the antigen binding domain comprises a variable domain comprising a CDR1 of SEQ ID NO: 69, CDR2 of SEQ ID NO: 70, and CDR3 of SEQ ID NO: 71.

65. The cell of claim 50, wherein the antigen binding domain comprises a variable domain comprising a CDR1 of SEQ ID NO: 73, CDR2 of SEQ ID NO: 74, and CDR3 of SEQ ID NO: 75.

66. The cell of claim 50, wherein the antigen binding domain comprises a variable domain comprising a CDR1 of SEQ ID NO: 77, CDR2 of SEQ ID NO: 78, and CDR3 of SEQ ID NO: 79.

67. The cell of any one of the preceding claims, further comprising a first nucleic acid encoding an inhibitory molecule that comprises a first polypeptide comprising at least a portion of an inhibitory molecule, associated with a second polypeptide comprising a positive signal from an intracellular signaling domain.

68. The cell of claim 67, wherein the inhibitory molecule comprises the first polypeptide comprising at least a portion of PD-1 and the second polypeptide comprising a costimulatory domain and primary signaling domain.

69. The cell of claim 67 or 68, wherein the recombinant nucleic acid comprises the first nucleic acid.

70. The cell of any one of the preceding claims, wherein the cell further comprises a second nucleic acid encoding IL-15.

71. The cell of claim 70, wherein the recombinant nucleic acid comprises the second nucleic acid.

72. The cell of claim 71, wherein IL-15 and the TFP are expressed in frame from the recombinant nucleic acid.

73. The cell of claim 72, wherein IL-15 and the TFP, when expressed in frame from the recombinant nucleic acid, are separated by a self-cleaving peptide, e.g., T2A.

74. The cell of any of claims 70-73, wherein expression of IL-15 increases persistence of the cells.

75. The cell of claim 68, wherein the second polypeptide comprises at least a portion of CD28.

76. The cell of claim 68 or 75, wherein the first polypeptide comprises an extracellular domain and a transmembrane domain of PD-1 linked to an intracellular domain of CD28.

77. The cell of any one of claims 1-76, further comprising a nucleic acid sequence encoding IL-15 polypeptide or a fragment thereof.

78. The cell of claim 77, wherein the IL-15 polypeptide is secreted when expressed in a T cell.

79. The cell of claim 77 or 78, wherein the IL-15 polypeptide comprises a sequence of SEQ ID NO: 81.

80. The cell of any one of claims 77-79, wherein the nucleic acid sequence further encodes an IL-15 receptor (IL-15R) subunit or a fragment thereof.

81. The cell of any one of claims 77-80, wherein the IL-15R subunit is IL-15R alpha (IL-15Rα).

82. The cell of claim 80 or 81, wherein the nucleic acid sequence encodes a fusion protein comprising the IL-15 polypeptide linked to the IL-15Rα subunit.

83. The cell of claim 82, wherein the fusion protein comprises a sequence of SEQ ID NO: 84.

84. The cell of claim 82 or 83, wherein the fusion protein is expressed on cell surface when expressed.

85. The cell of claim 82 or 83, wherein the fusion protein is secreted when expressed.

86. The cell of any one of claims 1-85, further comprising a first nucleic acid sequence encoding IL-15 receptor alpha (IL-15Rα) polypeptide or a fragment thereof.

87. The cell of claim 86, wherein the first nucleic acid sequence further encodes PD-1 or a fragment thereof.

88. The cell of claim 86 or 87, wherein the first nucleic acid sequence further encodes CD28 or a fragment thereof.

89. The cell of any one of claims 86-88, wherein the first nucleic acid sequence encodes a fusion protein comprising an extracellular domain and a transmembrane domain of PD-1 linked to an intracellular domain of CD28 linked to IL-15Rα.

90. The cell of claim 89, wherein the fusion protein comprises a sequence of SEQ ID NO: 85 or SEQ ID NO: 86.

91. The cell of any one of claims 86-90, further comprising a second nucleic acid sequence encoding an IL-15 polypeptide or a fragment thereof.

92. The cell of claim 91, wherein the IL-15 polypeptide or the fragment thereof is secreted when expressed.

93. The cell of any one of claims 73-92, wherein the antigen binding domain of the TFP is an anti-CD19 binding domain and the TFP has the sequence of SEQ ID NO: 31.

94. The cell of any one of claims 73-92, wherein the antigen binding domain of the TFP is an anti-MSLN binding domain and the TFP has the sequence of SEQ ID NO: 32.

95. The recombinant nucleic acid of any one of the preceding claims.

96. The recombinant nucleic acid of claim 95, wherein the nucleic acid is selected from the group consisting of a DNA and an RNA.

97. The recombinant nucleic acid of claim 95 or 96, wherein the nucleic acid is an mRNA.

98. The recombinant nucleic acid of claim 95 or 96, wherein the nucleic acid is circRNA.

99. The recombinant nucleic acid of any one of claims 95-97, wherein the recombinant nucleic acid comprises a nucleic acid analog, wherein the nucleic acid analog is not in an encoding sequence of the recombinant nucleic acid.

100. The recombinant nucleic acid of any one of claims 95-99, further comprising a leader sequence.

101. The recombinant nucleic acid of any one of claims 95-100, further comprising a promoter sequence.

102. The recombinant nucleic acid of any one of claims 95-101, further comprising a sequence encoding a poly(A) tail.

103. The recombinant nucleic acid of any one of claims 95-102, further comprising a 3′UTR sequence.

104. The recombinant nucleic acid of any one of claims 95-103, wherein the nucleic acid is an isolated nucleic acid or a non-naturally occurring nucleic acid.

105. The recombinant nucleic acid of any one of claims 95-104, wherein the nucleic acid is an in vitro transcribed nucleic acid.

106. A vector comprising the recombinant nucleic acid of any one of claims 95-105.

107. The vector of claim 106, wherein the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector.

108. The vector of claim 106 or 107, wherein the vector is an AAV6 vector.

109. The vector of any one of claims 106-108, further comprising a promoter.

110. The vector of any one of claims 106-109, wherein the vector comprises the recombinant nucleic acid of claim 93 or 94.

111. The vector of claim 110, wherein the vector comprises SEQ ID NOs: 29 or 30.

112. The vector of any one of claims 106-111, wherein the vector is an in vitro transcribed vector.

113. A circular RNA comprising the recombinant nucleic acid of any one of claims 95-105.

114. A pharmaceutical composition comprising:
(a) the cell of any one of claims 1-94; and
(b) a pharmaceutically acceptable carrier.

115. A method of producing a plurality of cells comprising the cell of claim 1 comprising:
(a) activating the γδ T cells isolated from PBMCs obtained from a donor by contacting the γδ T cells with (i) one or more immunomodulatory agents, and (ii) one or more cytokines;
(b) transducing the γδ T cells with the viral vector of any one of claims 106-112; and
(c) expanding the γδ T cells in the presence of the one or more cytokines.

116. The method of claim 115, wherein the method comprises obtaining the PBMCs from a donor.

117. The method of claim 115, wherein the method comprises isolating γδ T cells from the PBMCs.

118. The method of any one of claims 115-117, wherein the γδ T cells are obtained by negative selection.

119. The method of any one of claims 115-117, wherein γδ T cells are obtained by positive selection.

120. The method of any one of claims 115-119, wherein the one or more immunomodulatory agents comprise concanavalin, an anti-CD3 antibody, an anti-CD28 antibody, an anti-TCR delta antibody, an anti-TCR gamma antibody, or a pan Gamma delta TCR antibody.

121. The method of claim 115, wherein the anti-TCR delta antibody is an anti-TCR delta1 antibody.

122. The method of claim 115, wherein the anti-TCR gamma antibody is an anti-TCR gamma9 antibody.

123. The method of claim 115, wherein the anti-TCR delta antibody, the anti-TCR gamma antibody, the pan Gamma delta TCR antibody are plate bound.

124. The method of claim 115, wherein the one or more immunomodulatory agents comprise anti-CD3 and anti-CD28 antibodies, e.g., human anti-CD3 and anti-CD28 antibodies.

125. The method of claim 124, wherein the anti-CD3 and the anti-CD28 antibodies are bead bound.

126. The method of claim 124, wherein the anti-CD3 and the anti-CD28 antibodies are suspended in a matrix.

127. The method of any one of claims 115-126, wherein the one or more cytokines are IL-2, IL-4, IL-7 or IL-15.

128. The method of claim 127, wherein the one or more cytokines are IL-7 and IL-15.

129. The method of claim 127, wherein the one or more cytokines are IL-2, IL-7, and IL-15.

130. The method of claim 127, wherein the one or more cytokines are IL-2 and IL-4.

131. The method of any one of claims 115-130, wherein the one or more immunomodulatory agents further comprise a retronectin.

132. The method of claim 131, wherein the retronectin is plate bound.

133. The method of claim 115, wherein the one or more immunomodulatory agents comprise anti-CD3 and anti-CD28 antibodies, e.g., human anti-CD3 and anti-CD28 antibodies, and the one or more cytokines are IL-7 and IL-15.

134. The method of claim 115, wherein the one or more immunomodulatory agents comprise anti-CD3 and anti-CD28 antibodies, e.g., human anti-CD3 and anti-CD28 antibodies, and the one or more cytokines are IL-2, IL-7, and IL-15.

135. The method of claim 115, wherein the one or more immunomodulatory agents comprise a retronectin and anti-CD3 and anti-CD28 antibodies, e.g., human anti-CD3 and anti-CD28 antibodies, and the one or more cytokines are IL-7 and IL-15.

136. The method of any one of claims 133-135, wherein the anti-CD3 and the anti-CD28 antibodies are bead bound.

137. The method of any one of claims 133-135, wherein the anti-CD3 and the anti-CD28 antibodies are suspended in a matrix.

138. The method of claim 115, wherein the one or more immunomodulatory agents comprise the concanavalin and the one or more cytokines are IL-2 and IL-4.

139. The method of claim 115, wherein the one or more immunomodulatory agents comprise an anti-TCR delta1 antibody and the one or more cytokines are IL-2, IL-7, and IL-15.

140. The method of claim 115, wherein the one or more immunomodulatory agents comprise an anti-TCR gamma9 antibody and the one or more cytokines are IL-2, IL-7, and IL-15.

141. The method of claim 115, wherein the one or more immunomodulatory agents comprise a retronectin and the pan Gamma delta TCR antibody and the one or more cytokines are IL-2, IL-7, and IL-15.

142. The method of claim 115, wherein the one or more immunomodulatory agents comprise a retronectin and an anti-TCR gamma9 antibody and the one or more cytokines are IL-2, IL-7, and IL-15.

143. The method of any one of claims 115-142, wherein the cells are activated in the presence of the immunomodulatory agents for 2, 3, 4, or 5 days and then expanded in the absence of the immunomodulatory agents.

144. The method of any one of claims 115-143, wherein the immunomodulatory agent comprises an antigen-presenting cell (APC) that expresses a ligand of the antigen binding domain of the TFP.

145. The method of claim 144, wherein the γδ T cells is contacted with the APC prior to transduction in the presence or absence of one or more additional immunomodulatory agents.

146. The method of claim 144 or 145, wherein the γδ T cells is contacted with the APC after removal of the one or more additional immunomodulatory agents.

147. The method of claim 146, wherein the γδ T cells is contacted with the APC in the absence of the one or more immunomodulatory agents.

148. A method of treating cancer in a subject in need thereof, the method comprising administering to the subject a pharmaceutical composition comprising (a) a modified γδ T cell produced according to the method any one of claims 115-147, and (b) a pharmaceutically acceptable carrier.

149. A method of treating cancer in a subject in need thereof, the method comprising administering to the subject a therapeutically effective amount of the pharmaceutical composition of claim 114.

150. The method of claim 148 or 149, wherein less cytokines are released in the subject compared a subject administered an effective amount of an unmodified control γδ T cell.

151. The method of any one of claims 148-150, wherein less cytokines are released in the subject compared a subject administered an effective amount of a modified αβ T cell comprising the recombinant nucleic acid of any one of claims 95-105, the vector of any one of claims 106-112, or the circRNA of claim 113.

152. The method of claim 151, wherein the method comprises administering the pharmaceutical composition in combination with an agent that increases the efficacy of the pharmaceutical composition.

153. The method of claim 149, wherein the method comprises administering the pharmaceutical composition in combination with an agent that ameliorates one or more side effects associated with the pharmaceutical composition.

154. The method of any one of claims 148-153, wherein the cancer is a solid cancer, a lymphoma or a leukemia.

155. The method of any one of claims 148-154, wherein the cancer is selected from the group consisting of renal cell carcinoma, breast cancer, lung cancer, ovarian cancer, prostate cancer, colon cancer, cervical cancer, brain cancer, liver cancer, pancreatic cancer, kidney and stomach cancer.

156. The cell of any one of claims 1-94, the recombinant nucleic acid of any one of claims 95-105, the vector of any one of claims 106-112, or the circRNA of claim 113, or the pharmaceutical composition of claim 114, for use as a medicament or in the preparation of a medicament.


SEQUENCE LISTING (107 sequences)

SEQ ID NO: 1 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 3 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 4 - protein, 160 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 5 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 6 - protein, 150 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 7 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - DNA, 24 bp, Artificial Sequence
  Description of Artificial Sequence Synthetic oligonucleotide
agggcaagtcaggacattagtaaa

SEQ ID NO: 10 - protein, 8 aa, Artificial Sequence
  Description of Artificial Sequence Synthetic peptide
RASQDISK

SEQ ID NO: 11 - DNA, 21 bp, Artificial Sequence
  Description of Artificial Sequence Synthetic oligonucleotide
atctaccatacatcaagatta

SEQ ID NO: 12 - protein, 7 aa, Artificial Sequence
  Description of Artificial Sequence Synthetic peptide
IYHTSRL

SEQ ID NO: 13 - DNA, 27 bp, Artificial Sequence
  Description of Artificial Sequence Synthetic oligonucleotide
caacagggtaatacgcttccgtacacg

SEQ ID NO: 14 - protein, 9 aa, Artificial Sequence
  Description of Artificial Sequence Synthetic peptide
QQGNTLPYT

SEQ ID NO: 15 - DNA, 30 bp, Artificial Sequence
  Description of Artificial Sequence Synthetic oligonucleotide
ggggtctcattacccgactatggtgtaagc

SEQ ID NO: 16 - protein, 10 aa, Artificial Sequence
  Description of Artificial Sequence Synthetic peptide
GVSLPDYGVS

SEQ ID NO: 17 - DNA, 42 bp, Artificial Sequence
  Description of Artificial Sequence Synthetic oligonucleotide
gtaatatggggtagtgaaaccacatactataattcagctctc

SEQ ID NO: 18 - protein, 14 aa, Artificial Sequence
  Description of Artificial Sequence Synthetic peptide
VIWGSETTYYNSAL

SEQ ID NO: 19 - DNA, 36 bp, Artificial Sequence
  Description of Artificial Sequence Synthetic oligonucleotide
cattattactacggtggtagctatgctatggactac

SEQ ID NO: 20 - protein, 12 aa, Artificial Sequence
  Description of Artificial Sequence Synthetic peptide
HYYYGGSYAMDY

SEQ ID NO: 21 - DNA, 321 bp, Artificial Sequence
  Description of Artificial Sequence Synthetic polynucleotide
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 22 - protein, 107 aa, Artificial Sequence
  Description of Artificial Sequence Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 23 - DNA, 360 bp, Artificial Sequence
  Description of Artificial Sequence Synthetic polynucleotide
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 24 - protein, 120 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 25 - protein, 111 aa, Artificial Sequence
  Description of Artificial Sequence Synthetic polypeptide
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 26 - protein, 119 aa, Artificial Sequence
  Description of Artificial Sequence Synthetic polypeptide
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 27 - protein, 10 aa, Artificial Sequence
  Description of Artificial Sequence Synthetic peptide
GGGGSGGGGS

SEQ ID NO: 28 - DNA, 30 bp, Artificial Sequence
  Description of Artificial Sequence Synthetic oligonucleotide
ggtggcggaggttctggaggtggaggttcc

SEQ ID NO: 29 - DNA, 9434 bp, Artificial Sequence
  Description of Artificial Sequence Synthetic polynucleotide
tgatcataatcaagccatatcacatctgtagaggtttacttgctttaaaaaacctccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgtcgacacgaagagacgactgactgactgactggaaagaggaagggctggaagaggaaggagcttgatccagatcccgatctcgatccagatccggatcgcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgttgctaggttactgtcatgagcggatacatatttgaatgtatttagaaaaataaacaaaagagtttgtagaaacgcaaaaaggccatccgtcaggatggccttctgcttaatttgatcggtggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatgggttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagtgacattaacctataaaaataggcgtatcacgaggccagcttgggaaaccataagaccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgatcgagatcgtgatccggatcaagatccagatcgaattggaggctacagtcagtggagaggactttcactgactgactgactgcgtctcaacctcctaggggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagtagggtgcgagagcgtcggtattaagcgggggagaattagataaatgggaaaaaattcggttaaggccagggggaaagaaacaatataaactaaaacatatagttagggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcgatcttcagacctggaggaggcaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggaccacgcgtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaagctgagcatccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctcagagccgccaccatgcttctgcttgtgacgtccctcctgctttgtgaactcccgcatcctgccttcctgctcatcccggaagttcagctggtagagtccggtggcgggctggtacagccagggggatctctccgtctctcatgtgctgccagtgggggagattggtccgctaactttatgtactggtatcgtcaagcacctgggaaacaacgcgagctggttgcacggatctctggacgaggggtagtggattacgtggaaagtgtgaaaggacgattcacaatttccagggacaattctaaaaacaccctttatttgcaaatgaatagtctgagagcagaagacacagccgtatattattgtgcagtcgcatcctattgggggcaggggactcttgtcacagtctcgagtgctgcagcaggcggcgggggctcaggagggggtgggtctggaggaggtgggagtctggaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatcgggtcaggcgagggcagaggaagtctgctaacatgcggtgacgtcgaggagaatcctggacctatgagaatcagcaagcctcacctgagaagcatctccattcagtgctacctgtgcctccttctgaacagccacttcctgaccgaggctggcattcacgtgttcatcctgggctgcttcagcgcaggactgcccaagaccgaggctaactgggttaacgtgatcagcgacctgaagaaaatcgaggacctgattcagagcatgcacatcgacgctaccctgtacaccgagagcgatgtgcatccttcttgcaaggtgacagccatgaagtgcttcctgctggaactccaggtgatctctctggagagcggagatgccagcattcacgacacagtggaaaacctgatcatcctggccaataactccctgtctagcaacggaaatgtgaccgaaagcggctgtaaagagtgcgaagagctggaggaaaagaacatcaaagagttcctgcagagcttcgtgcatatcgtgcagatgttcatcaacacctcttgagatatcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcagtactggtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaa

SEQ ID NO: 30 - DNA, 9881 bp, Artificial Sequence
  Description of Artificial Sequence Synthetic polynucleotide
tgatcataatcaagccatatcacatctgtagaggtttacttgctttaaaaaacctccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgtcgacacgaagagacgactgactgactgactggaaagaggaagggctggaagaggaaggagcttgatccagatcccgatctcgatccagatccggatcgcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcagcttgggaaaccataagagctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcacatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacaacgtggctttcccccccccccccatgacattaacctataaaaataggcgtatcacgaggccagcttgggaaaccataagaccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgatcgagatcgtgatccggatcaagatccagatcgaattggaggctacagtcagtggagaggactttcactgactgactgactgcgtctcaacctcctaggggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagtagggtgcgagagcgtcggtattaagcgggggagaattagataaatgggaaaaaattcggttaaggccagggggaaagaaacaatataaactaaaacatatagttagggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcgatcttcagacctggaggaggcaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggaccacgcgtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaagctgagcatccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctcagagccgccaccatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagctgccgcaggaggcggtggcagtggtgggggaggatctggcggaggaggtagcttggaagatggtaatgaagaaatgggtggtattacacagacaccatataaagtctccatctctggaaccacagtaatattgacatgccctcagtatcctggatctgaaatactatggcaacacaacgataagaatataggaggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatcgggtcaggcgagggcagaggaagtctgctaacatgcggtgacgtcgaggagaatcctggacctatgagaatcagcaagcctcacctgagaagcatctccattcagtgctacctgtgcctccttctgaacagccacttcctgaccgaggctggcattcacgtgttcatcctgggctgcttcagcgcaggactgcccaagaccgaggctaactgggttaacgtgatcagcgacctgaagaaaatcgaggacctgattcagagcatgcacatcgacgctaccctgtacaccgagagcgatgtgcatccttcttgcaaggtgacagccatgaagtgcttcctgctggaactccaggtgatctctctggagagcggagatgccagcattcacgacacagtggaaaacctgatcatcctggccaataactccctgtctagcaacggaaatgtgaccgaaagcggctgtaaagagtgcgaagagctggaggaaaagaacatcaaagagttcctgcagagcttcgtgcatatcgtgcagatgttcatcaacacctcttgagatatcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcagtactggtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaa

SEQ ID NO: 31 - protein, 655 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - protein, 521 aa, Artificial Sequence
  Description of Artificial Sequence Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 33 - protein, 469 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 34 - protein, 338 aa, Artificial Sequence
  Description of Artificial Sequence Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 35 - protein, 10 aa, Artificial Sequence
  Description of Artificial Sequence Synthetic peptide
GGDWSANFMY

SEQ ID NO: 36 - protein, 19 aa, Artificial Sequence
  Description of Artificial Sequence Synthetic peptide
RISGRGVVDYVESVKGRFT

SEQ ID NO: 37 - protein, 3 aa, Artificial Sequence
  Description of Artificial Sequence Synthetic peptide
ASY

SEQ ID NO: 38 - protein, 10 aa, Artificial Sequence
  Description of Artificial Sequence Synthetic peptide
GSTSSINTMY

SEQ ID NO: 39 - protein, 19 aa, Artificial Sequence
  Description of Artificial Sequence Synthetic peptide
FISSGGSTNVRDSVKGRFT

SEQ ID NO: 40 - protein, 11 aa, Artificial Sequence
  Description of Artificial Sequence Synthetic peptide
YIPYGGTLHDF

SEQ ID NO: 41 - protein, 116 aa, Artificial Sequence
  Description of Artificial Sequence Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 42 - protein, 10 aa, Artificial Sequence
  Description of Artificial Sequence Synthetic peptide
GSTFSIRAMR

SEQ ID NO: 43 - protein, 18 aa, Artificial Sequence
  Description of Artificial Sequence Synthetic peptide
VIYGSSTYYADAVKGRFT

SEQ ID NO: 44 - protein, 9 aa, Artificial Sequence
  Description of Artificial Sequence Synthetic peptide
DTIGTARDY

SEQ ID NO: 45 - protein, 9 aa, Artificial Sequence
  Description of Artificial Sequence Synthetic peptide
SIFDIVRGS

SEQ ID NO: 46 - protein, 12 aa, Artificial Sequence
  Description of Artificial Sequence Synthetic peptide
AIITSGGATNYA

SEQ ID NO: 47 - protein, 12 aa, Artificial Sequence
  Description of Artificial Sequence Synthetic peptide
CNMESVRYRNYW

SEQ ID NO: 48 - protein, 116 aa, Artificial Sequence
  Description of Artificial Sequence Synthetic polypeptide
QVQLQESGGGLVQTGGSLRLSCTASGSIFDIVRGSWYRQAPGNQRELVAIITSGGATNYADSVAGRFTISRDSAWKALYLQMNSLKPEDTAVYFCNMESVRYRNYWGQGTQVTVSS

SEQ ID NO: 49 - protein, 9 aa, Artificial Sequence
  Description of Artificial Sequence Synthetic peptide
FTLEHYSIG

SEQ ID NO: 50 - protein, 13 aa, Artificial Sequence
  Description of Artificial Sequence Synthetic peptide
SCITSSGGIPKYA

SEQ ID NO: 51 - protein, 21 aa, Artificial Sequence
  Description of Artificial Sequence Synthetic peptide
CGAATPDDDCSVPGHYGLNYW

SEQ ID NO: 52 - protein, 126 aa, Artificial Sequence
  Description of Artificial Sequence Synthetic polypeptide
QVQLQESGGGLVQPGGSLRLSCAASGFTLEHYSIGWFRQAPGKDLEGVSCITSSGGIPKYADSVKGRFIISRDNAKNTGYLQMNSLKPEDTAVYYCGAATPDDDCSVPGHYGLNYWGKGTQVTVSS

SEQ ID NO: 53 - protein, 9 aa, Artificial Sequence
  Description of Artificial Sequence Synthetic peptide
FTFDAYAIG

SEQ ID NO: 54 - protein, 13 aa, Artificial Sequence
  Description of Artificial Sequence Synthetic peptide
ICLSPSDGSTYYA

SEQ ID NO: 55 - protein, 16 aa, Artificial Sequence
  Description of Artificial Sequence Synthetic peptide
CATPSWCSLKADFGSW

SEQ ID NO: 56 - protein, 121 aa, Artificial Sequence
  Description of Artificial Sequence Synthetic polypeptide
QVQLQESGGGLVQAGGSLRLSCAAPGFTFDAYAIGWFRQAPGKEREGVICLSPSDGSTYYADSVKGRFTISSDNAKNTVYLQMNSLKPEDTAVYYCATPSWCSLKADFGSWGQGTQVTVSS

SEQ ID NO: 57 - protein, 9 aa, Artificial Sequence
  Description of Artificial Sequence Synthetic peptide
SIFSATRME

SEQ ID NO: 58 - protein, 12 aa, Artificial Sequence
  Description of Artificial Sequence Synthetic peptide
AIVTSGGRTNYA

SEQ ID NO: 59 - protein, 12 aa, Artificial Sequence
  Description of Artificial Sequence Synthetic peptide
CKFERYDYVNYW

SEQ ID NO: 60 - protein, 116 aa, Artificial Sequence
  Description of Artificial Sequence Synthetic polypeptide
QVQLQESGGGLVQPGGSLRLSCTASGSIFSATRMEWYRQAPGKQRELVAIVTSGGRTNYADSVNGRFTISRDNAKNTLYLQMNNLKPEDTAVYYCKFERYDYVNYWGRGTQVTVSS

SEQ ID NO: 61 - protein, 9 aa, Artificial Sequence
  Description of Artificial Sequence Synthetic peptide
SIFSIARMN

SEQ ID NO: 62 - protein, 12 aa, Artificial Sequence
  Description of Artificial Sequence Synthetic peptide
AILNRAGRTDYA

SEQ ID NO: 63 - protein, 12 aa, Artificial Sequence
  Description of Artificial Sequence Synthetic peptide
CNLQTISYHDFW

SEQ ID NO: 64 - protein, 116 aa, Artificial Sequence
  Description of Artificial Sequence Synthetic polypeptide
QVQLQESGGGLVQPGGSLRLSCVASGSIFSIARMNWYRQAPGKQRELVAILNRAGRTDYADSVKGRFTISSDNAKTTVYLQMNSLKPEDTALYYCNLQTISYHDFWGQGTQVTVSS

SEQ ID NO: 65 - protein, 9 aa, Artificial Sequence
  Description of Artificial Sequence Synthetic peptide
SIFDIARGN

SEQ ID NO: 66 - protein, 12 aa, Artificial Sequence
  Description of Artificial Sequence Synthetic peptide
AIITSGGATNYA

SEQ ID NO: 67 - protein, 12 aa, Artificial Sequence
  Description of Artificial Sequence Synthetic peptide
CNMESLSYRHYW

SEQ ID NO: 68 - protein, 116 aa, Artificial Sequence
  Description of Artificial Sequence Synthetic polypeptide
QVQLQESGGGLVQTGGSLRLSCTASGSIFDIARGNWYRQAPGKQRELVAIITSGGATNYADSVAGRFTISRDDAKNTVYLQMNGLKPEDTAVYFCNMESLSYRHYWGQGTQVTVSS

SEQ ID NO: 69 - protein, 9 aa, Artificial Sequence
  Description of Artificial Sequence Synthetic peptide
SIIRDNVMA

SEQ ID NO: 70 - protein, 12 aa, Artificial Sequence
  Description of Artificial Sequence Synthetic peptide
AIINTGGSANVD

SEQ ID NO: 71 - protein, 9 aa, Artificial Sequence
  Description of Artificial Sequence Synthetic peptide
CNVYYRDLW

SEQ ID NO: 72 - protein, 113 aa, Artificial Sequence
  Description of Artificial Sequence Synthetic polypeptide
QVQLQESGGGLVQAGGSLRLSCAASKSIIRDNVMAWHRQAPGKQRELVAIINTGGSANVDDSVKGRFTISRDNAKNMVYLQMNNLKPEDTAVYYCNVYYRDLWGQGTQVTVSS

SEQ ID NO: 73 - protein, 9 aa, Artificial Sequence
  Description of Artificial Sequence Synthetic peptide
FTLDRYAVG

SEQ ID NO: 74 - protein, 13 aa, Artificial Sequence
  Description of Artificial Sequence Synthetic peptide
SCISSSGDIIKYA

SEQ ID NO: 75 - protein, 21 aa, Artificial Sequence
  Description of Artificial Sequence Synthetic peptide
CTAADPKDDCSVPGYYGLNYW

SEQ ID NO: 76 - protein, 126 aa, Artificial Sequence
  Description of Artificial Sequence Synthetic polypeptide
QVQLQESGGGLVQPGGSLRLSCVASGFTLDRYAVGWFRQAPGKELEGVSCISSSGDIIKYADSAKGRFTIARDNAKNTAYLQMNSLKPEDTAVYYCTAADPKDDCSVPGYYGLNYWGKGTQVTVSS

SEQ ID NO: 77 - protein, 9 aa, Artificial Sequence
  Description of Artificial Sequence Synthetic peptide
FTLDKYAIG

SEQ ID NO: 78 - protein, 13 aa, Artificial Sequence
  Description of Artificial Sequence Synthetic peptide
SCITSSSGVVKYA

SEQ ID NO: 79 - protein, 21 aa, Artificial Sequence
  Description of Artificial Sequence Synthetic peptide
CAAAGPPDDCSVPGYYGLNYW

SEQ ID NO: 80 - protein, 126 aa, Artificial Sequence
  Description of Artificial Sequence Synthetic polypeptide
QVQLQESGGGLVQPGGSLRLSCVASGFTLDKYAIGWFRQAPGKELEGVSCITSSSGVVKYADSVKGRFIISRDNTNNRAFLQMSSLKPEDTAVYYCAAAGPPDDCSVPGYYGLNYWGKGTQVTVSS

SEQ ID NO: 81 - protein, 133 aa, Artificial Sequence
  Description of Artificial Sequence Synthetic polypeptide
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 82 - protein, 808 aa, Artificial Sequence
  Description of Artificial Sequence Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLDYKDDDDKDYKDDDDKDYKDDDDK

SEQ ID NO: 83 - protein, 631 aa, Artificial Sequence
  Description of Artificial Sequence Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPGMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAYRSKSRQTPPLASVEMEAMEALPVTWGTSSRDEDLENCSHHL

SEQ ID NO: 84 - protein, 396 aa, Artificial Sequence
  Description of Artificial Sequence Synthetic polypeptide
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 85 - protein, 251 aa, Artificial Sequence
  Description of Artificial Sequence Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAYRSKSRQTPPLASVEMEAMEALPVTWGTSSRDEDLENCSHHL

SEQ ID NO: 86 - protein, 212 aa, Artificial Sequence
  Description of Artificial Sequence Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAYRS

SEQ ID NO: 87 - protein, 172 aa, Artificial Sequence
  Description of Artificial Sequence Synthetic polypeptide
DPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 88 - protein, 814 aa, Artificial Sequence
  Description of Artificial Sequence Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPGMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAYRSKSRQTPPLASVEMEAMEALPVTWGTSSRDEDLENCSHHLGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 89 - protein, 722 aa, Artificial Sequence
  Description of Artificial Sequence Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 90 - protein, 521 aa, Artificial Sequence; identical to SEQ ID NO: 32
  Description of Artificial Sequence Synthetic polypeptide

SEQ ID NO: 91 - protein, 20 aa, Artificial Sequence
  Description of Artificial Sequence Synthetic peptide
  SITE (1)..(20): This sequence may encompass 1-4 “Gly Gly Gly Gly Ser” repeating units
GGGGSGGGGSGGGGSGGGGS

SEQ ID NO: 92 - protein, 4 aa, Artificial Sequence
  Description of Artificial Sequence Synthetic peptide
  See specification as filed for detailed description of substitutions and preferred embodiments
GGGS

SEQ ID NO: 93 - protein, 20 aa, Artificial Sequence
  Description of Artificial Sequence Synthetic peptide
GGGGSGGGGSGGGGSGGGGS

SEQ ID NO: 94 - protein, 15 aa, Artificial Sequence
  Description of Artificial Sequence Synthetic peptide
GGGGSGGGGSGGGGS

SEQ ID NO: 95 - protein, 4 aa, Artificial Sequence
  Description of Artificial Sequence Synthetic peptide
GGGS

SEQ ID NO: 96 - protein, 20 aa, Artificial Sequence
  Description of Artificial Sequence Synthetic peptide
  SITE (1)..(20): This sequence may encompass 2-4 “Gly Gly Gly Gly Ser” repeating units
GGGGSGGGGSGGGGSGGGGS

SEQ ID NO: 97 - protein, 15 aa, Artificial Sequence
  Description of Artificial Sequence Synthetic peptide
  SITE (1)..(15): This sequence may encompass 1-3 “Gly Gly Gly Gly Ser” repeating units
GGGGSGGGGSGGGGS

SEQ ID NO: 98 - RNA, 5000 nt, Artificial Sequence
  Description of Artificial Sequence Synthetic polynucleotide
  SITE (1)..(5000): This sequence may encompass 50-5000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 99 - protein, 30 aa, Artificial Sequence
  Description of Artificial Sequence Synthetic polypeptide
  SITE (1)..(30): This sequence may encompass 1-6 “Gly Gly Gly Gly Ser” repeating units
GGGGSGGGGSGGGGSGGGGSGGGGSGGGGS

SEQ ID NO: 100 - protein, 50 aa, Artificial Sequence
  Description of Artificial Sequence Synthetic polypeptide
  SITE (1)..(50): This sequence may encompass 1-10 “Gly Gly Gly Gly Ser” repeating units
  See specification as filed for detailed description of substitutions and preferred embodiments
GGGGSGGGGSGGGGSGGGGSGGGGSGGGGSGGGGSGGGGSGGGGSGGGGS

SEQ ID NO: 101 - RNA, 2000 nt, Artificial Sequence
  Description of Artificial Sequence Synthetic polynucleotide
  SITE (1)..(2000): This sequence may encompass 50-2000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 102 - DNA, 5000 bp, Artificial Sequence
  Description of Artificial Sequence Synthetic polynucleotide
  SITE (1)..(5000): This sequence may encompass 50-5000 nucleotides
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 103 - RNA, 5000 nt, Artificial Sequence; identical to SEQ ID NO: 98
  Description of Artificial Sequence Synthetic polynucleotide
  SITE (1)..(5000): This sequence may encompass 100-5000 nucleotides

SEQ ID NO: 104 - RNA, 400 nt, Artificial Sequence
  Description of Artificial Sequence Synthetic polynucleotide
  SITE (1)..(400): This sequence may encompass 100-400 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 105 - protein, 25 aa, Artificial Sequence
  Description of Artificial Sequence Synthetic peptide
  SITE (1)..(25): This sequence may encompass 1-5 “Gly Gly Gly Gly Ser” repeating units
GGGGSGGGGSGGGGSGGGGSGGGGS

SEQ ID NO: 106 - protein, 5 aa, Artificial Sequence
  Description of Artificial Sequence Synthetic peptide
  See specification as filed for detailed description of substitutions and preferred embodiments
GGGGS

SEQ ID NO: 107 - DNA, 100 bp, Artificial Sequence
  Description of Artificial Sequence Synthetic polynucleotide
tttttttttttttttttttttttttttttttttttttttttttttttttttttttttttttttttttttttttttttttttttttttttttttttttttt